(19) United States
(12) Patent Application Publication
(10) Pub. No.: US 2018/0296561 A1
(43) Pub. Date: Oct. 18, 2018

(54) The Methods For Treatment Of Tumors

(71) Applicant: The University of North Carolina at Chapel Hill, Chapel Hill, NC (US)

(72) Inventor: Henry Shelton Earp, III, Chapel Hill, NC (US)

(73) Assignee: The University of North Carolina at Chapel Hill, Chapel Hill, NC (US)

(21) Appl. No.: 15/766,612

(22) PCT Filed: Oct. 7, 2016
(86) PCT No.: PCT/US2016/056040

§ 371 Date: Apr. 6, 2018

Related U.S. Application Data
(60) Provisional application No. 62/238,276, filed on Oct. 7, 2015.
(60) Provisional application No. 62/238,548, filed on Oct. 7, 2015.

(51) Int. Cl. A61K 31/519; A61K 31/5377; A61K 31/551; A61K 45/06; A61P 35/00; C07K 16/28; A61K 39/395

(52) U.S. Cl. CPC A61K 31/519; A61K 31/5377; A61K 31/551; A61K 2039/505; A61P 35/00; C07K 16/2818; A61K 39/39541; A61K 45/06

(57) ABSTRACT

This invention is in the area of improved therapeutic combinations for and methods of treating selected cancers using specific Mer tyrosine kinase (MerTK) inhibitors in combination with immune checkpoint inhibitors. In one aspect, an improved treatment for select cancers is disclosed using specific Mer tyrosine kinase (MerTK) inhibitors, for example UNC2371, in combination with an immune checkpoint inhibitor, for example, a cytotoxic T-lymphocyte-associated protein 4 (CTLA4) inhibitor, a programmed cell death protein 1 (PD1) inhibitor, or a programmed death-ligand 1 (PDL-1) inhibitor.

### RELATED APPLICATION DATA

This application claims the benefit of U.S. Provisional Patent Application Ser. No. 62/238,276, filed Oct. 7, 2015 and U.S. Provisional Patent Application Ser. No. 62/238,548, filed Oct. 7, 2015, the disclosures of each of which are incorporated herein by reference in their entirety.

### STATEMENT OF GOVERNMENT SUPPORT

This invention was made with government support under grant number HHSN261200800001E awarded by the National Institutes of Health. The government has certain rights in this invention.

### FIELD OF THE INVENTION

This invention is in the area of improved therapeutic combinations for and methods of treating selected cancers using specific Mer tyrosine kinase (MerTK) inhibitors in combination with immune checkpoint inhibitors. In one aspect, an improved treatment for select cancers is disclosed using specific Mer tyrosine kinase (MerTK) inhibitors, for example UNC2371, in combination with an immune checkpoint inhibitor, for example, a cytotoxic T-lymphocyte-associated protein 4 (CTLA4) inhibitor, a programmed cell death protein 1 (PD1) inhibitor, or a programmed death-ligand 1 (PDL-1) inhibitor, or combination thereof.

### BACKGROUND OF THE INVENTION

### Mer Tyrosine Kinase

Mer Tyrosine Kinase (MerTK) is a member of a receptor tyrosine kinase (RTK) family known as TAM, which also includes AXL and TYRO3. Each member of the TAM family contains an extracellular domain, a transmembrane domain and a conserved intracellular kinase domain. MerTK was first discovered in the laboratory of H. Shelton Earp at the University of North Carolina in 1994 (Graham et al., Cloning and mRNA expression analysis of a novel human proto-oncogene, c-mer. Cell Growth Differ 5, 647-657 (1994)). The TAM family members undergo ligand-induced homodimerization, followed by catalytic tyrosine kinase activation and intracellular signaling. Cross-phosphorylation has also been demonstrated within this RTK family, suggesting heterodimerization can occur also. These RTKs are widely expressed in many epithelial tissues and in cells of the immune, nervous, and reproductive systems. MerTK was given its name by the Earp laboratory because it was found to be expressed in monocytes and in tissues of epithelial and reproductive tissue.

Since the discovery of MerTK in the Earp laboratory in 1994, there has been a growing body of literature and patents that indicates the possibility of MerTK as a drugable target for a number of indications, including the treatment of cancer. MerTK is ectopically expressed or overexpressed in a number of hematologic and epithelial malignant cells. Expression of MerTK correlates with poor prognosis and/or chemoresistance in these tumor types. The mechanisms by which increased MerTK signaling in tumor cells contributes to tumor malignancy, however, remain unclear.

### Inhibition of MerTK

A number of small molecule inhibitors of MerTK have been previously described. WO2013/052417 titled “Pyrrolopyrimidine Compounds for the Treatment of Cancer” filed by Wang, et al., and assigned to the University of North Carolina describes pyrrolopyrimidines with MerTK inhibitory activity for the treatment of tumors such as myeloid leukemia, lymphoblastic leukemia, melanoma, breast, lung, colon, liver, gastric, kidney, ovarian, uterine and brain cancer, wherein the pyrrolopyrimidines have the general structures below, with R substituents as defined in the those applications:

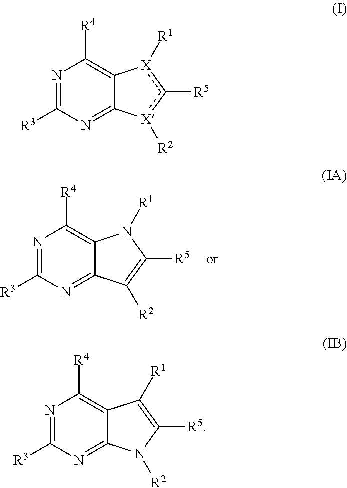

In addition to the formulas described above, WO2013/052417 also specifically describes the MerTK inhibitor UNC2371 (See WO2013/052417, Compound 20, pg. 27):

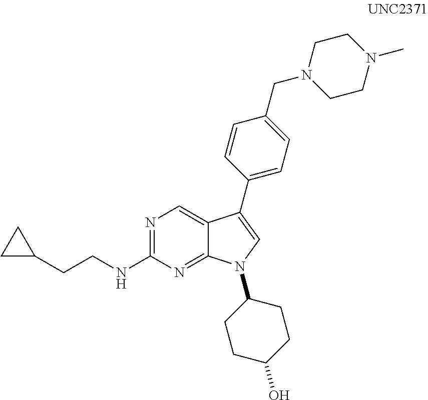

In May 2013, Dr. Shelton Earp, in an academic presentation at the Lineberger Comprehensive Cancer Center, raised the issue of whether combining a MerTK inhibitor with an immune checkpoint inhibitor would have any therapeutic value, without answering the question. See Earp, S. “Mer Tyrosine Kinase: Role in Oncogenesis and Immune Response”, Presentation at the Lineberger Comprehensive Cancer Center Mer Symposium, May 2013. In November 2013, Dr. Stephen Frye, in an academic presentation at Northwestern University, also raised this issue but did not answer the question, and presented only data on monotherapies using the MerTK inhibitor UNC2025. See Frye, S. “Academic Drug Discovery and Chemical Biology”, Presentation at the Northwestern 18th Annual Drug Discovery Symposium, November 2013. The structure of the pyrrolopyrimidine compound UNC2025 is:

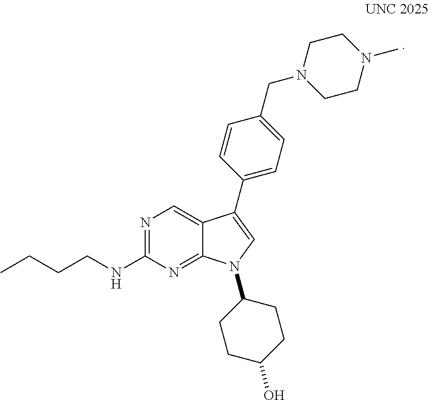

WO2011/146313 and WO2014/062774, both titled “Pyrazolopyrimidine Compounds for the Treatment of Cancer” filed by Wang, et al., and assigned to the University of North Carolina describe pyrazolopyrimidines with MerTK inhibitory activity for the treatment of tumors such as myeloid leukemia, lymphoblastic leukemia, melanoma, breast, lung, colon, liver, gastric, kidney, ovarian, uterine and brain cancer, wherein the pyrazolopyrimidines have the general structures below, with R substituents as defined in the those applications:

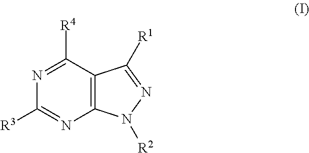

In January 2012, Liu, J, et al., published a comparison of the activity of forty four pyrazolopyrimidine compounds against MerTK, Axl and Tyro3 kinases. One of these compounds (UNC569) was tested for inhibition of MerTK autophosphorylation in human B-ALL cells (“Discovery of Novel Small Molecule Mer Kinase Inhibitors for the Treatment of Pediatric Acute Lymphoblastic Leukemia.” ACS Med Chem Lett. 2012 Feb 9;3(2):129-134.). In May 2013, Schlegel, et al., published results on the pyrazolopyrimidine compound UNC1062, which reduced activation of MERTK-mediated downstream signaling, induced apoptosis in culture, reduced colony formation in soft agar, and inhibited invasion of melanoma cells (“MER receptor tyrosine kinase is a therapeutic target in melanoma.” J Clin Invest. 2013 May;123(5):2257-67).

In July 2013, Liu, J, et al. published evidence of anti-tumor activity mediated by a member of this novel class of pyrazolopyrimidine inhibitors, UNC1062 which inhibited MerTK phosphorylation and colony formation in soft agar (“UNC1062, a new and potent Mer inhibitor.” Eur J Med Chem. 2013 July;65:83-93). In November 2013, Christoph, S. et al., published the effect of a pyrazolopyrimidine (UNC569) in ALL and ATRT (atypical teratoid/rhabdoid tumors (ATRT)) (“UNC569, a novel small-molecule Mer inhibitor with efficacy against acute lymphoblastic leukemia in vitro and in vivo.” Mol Cancer Ther. 2013 November; 12(11):2367-77). The MerTK inhibitors UNC569 and UNC1062 have the following structures:

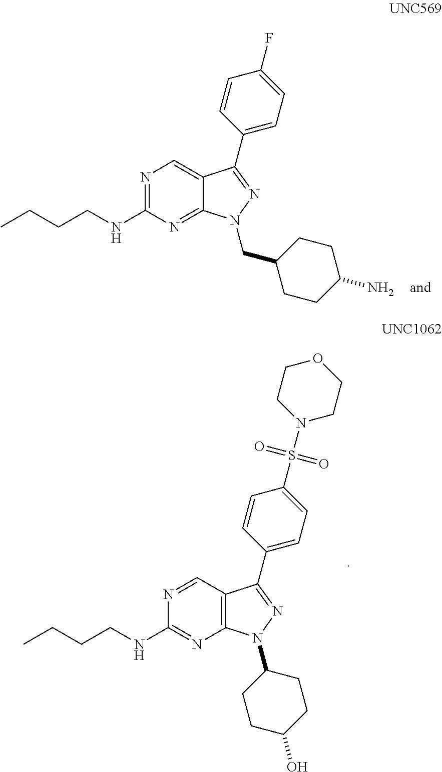

WO2013/177168 and WO2014/085225, both titled “Pyrimidine Compounds for the Treatment of Cancer” filed by Wang, et al., and assigned to the University of North Carolina describe pyrimidines with MerTK inhibitory activity for the treatment of tumors such as myeloid leukemia, lymphoblastic leukemia, melanoma, breast, lung, colon, liver, gastric, kidney, ovarian, uterine and brain cancer, wherein the pyrimidines have the general structures below, with R substituents as defined in the those applications:

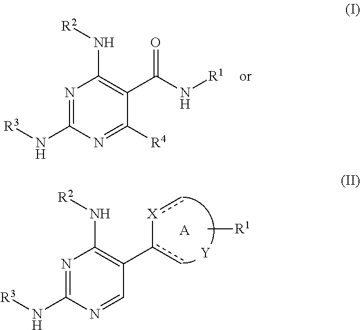

In December 2013, Zhang, W., et al., published a comparison of the activity of forty six 5-arylpyrimidine based compounds for treatment of tumors (“Pseudo-cyclization through intramolecular hydrogen bond enables discovery of pyridine substituted pyrimidines as new Mer kinase inhibitors.” J. Med. Chem., vol. 56:9683-9692, 2013). These pyrimidine compounds were identified using a pseudo-ring replacement strategy based on the previously identified pyrazolopyrimidine MerTK inhibitor, UNC569.

An important observation was made in 2013 that MerTK −/− knock-out mice are less susceptible to tumor growth than normal mice. MerTK is normally expressed in myeloid lineage cells where it acts to suppress pro-inflammatory cytokines following ingestion of apoptotic material. It was found that MerTK −/− leukocytes exhibit lower tumor cell-induced expression of wound healing cytokines (IL-10 and GAS6) and enhanced expression of acute inflammatory cytokines (IL-12 and IL-6). Further, intratumoral CD8+ lymphocytes are increased. The loss of MerTK in the tumor microenvironment in Mer−/− mice slowed the establishment, growth, and metastasis of mammary tumors and melanomas in immune competent, syngeneic mice. Cook, R. S. et al., MerTK inhibition in tumor leukocytes decreases tumor growth and metastasis, J Clin Invest 123, 3231-3242 (2013).

Linger et al. have also presented data demonstrating increased MerTK expression in E2A-PBX11 and other cytogenetic subgroups of B-acute lymphoblastic leukemia (B-ALL), and that MerTK inhibition may attenuate prosurvival and proliferation signaling Linger et al., Mer receptor tyrosine kinase is a therapeutic target in pre-B-cell acute lymphoblastic leukemia, Blood, vol. 122(9):1599-1609, 2013.

Zhang et al. (“UNC2025, a Potent and Orally Bioavailable MER/FLT3 Dual Inhibitor”, J Med Chem, 57:7031-7041, 2014) have reported that UNC2025 (a pyrrolopyrimidine, see structure above) has dual inhibitory activity against MerTK and FLT3 and can inhibit colony formation in non-small cell lung cancer (NSCLC) and acute myeloid leukemia (AML) tumor cell lines. Lee-Sherick, et al. (“Efficacy of a Mer and Flt3 tyrosine kinase small molecule inhibitor, UNC1666, in acute myeloid leukemia”, Oncotarget, Advance Publications 2015 Feb 10, 2015) have reported that UNC 1666 (a pyrrolopyrimidine) decreases oncogenic signaling and myeloid survival in AML.

Paolino et al. have reported on the treatment of wild-type NK cells with a small molecule TAM kinase inhibitor—LDC1267—that conferred therapeutic potential and efficiently enhanced anti-metastatic NK cell activity in vivo. Oral or intraperitoneal administration of this TAM inhibitor markedly reduced murine mammary cancer and melanoma metastases dependent on NK cells. See, Paolino, M., et al., The E3 ligase Cbl-b and TAM receptors regulate cancer metastasis via natural killer cells, Nature, vol. 507:508-512, 2014. LDC1267 is a highly selective TAM kinase inhibitor with IC₅₀ of <5 nM, 8 nM, and 29 nM for MerTK, Tyro3, and Axl, respectively, and has the chemical structure:

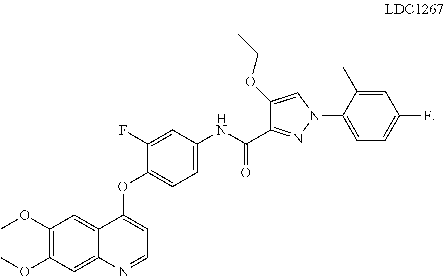 [SMILES: CCOc1cn(-c2ccc(F)cc2C)nc1C(=O)Nc1ccc(Oc2ccnc3cc(OC)c(OC)cc23)c(F)c1]

Bernsmeier, et al., have noted that characteristics of decompensated cirrhosis and acute-on-chronic liver failure (ACLF) include susceptibility to infection, immune paresis and monocyte dysfunction. The authors found that the number of monocytes and macrophages that expressed MerTK was greatly increased in circulation, livers and lymph nodes of patients with ACLF. They found that addition of a substituted pyrazolopyrimidine UNC569 (see WO 2011/146313 filed by Wang, et al., and assigned to University of North Carolina at Chapel Hill, page 25) restored production of inflammatory cytokines. Bernsmeier, et al., “Patients with Acute-on-Chronic Liver Failure Have Increased Numbers of Regulatory Immune Cells Expressing the Receptor Tyrosine Kinase MERTK”, Gastroenterology 2015; 1-13.

PCT/US2015/024380 titled “Therapeutic Uses of Selected Pyrimidine Compounds with Anti-Mer Tyrosine Kinase Activity”, PCT/US2015/024381 titled “Therapeutic Uses of Selected Pyrrolopyrimidine Compounds with Anti-Mer Tyrosine Kinase Activity”, and PCT/US2015/024373 titled “Therapeutic Uses of Selected Pyrazolopyrimidine Compounds with Anti-Mer Tyrosine Kinase Activity”, all filed by Wang, et al. in April 2015 and assigned to the University of North Carolina, describe pyrimidines, pyrrolopyrimidines, and pyrazolopyrimidines, respectively, with MerTK inhibitory activity. PCT US2015/024301 titled “MerTK-Specific Pyrimidine Compounds”, PCT/US2015/024362 titled “MerTK-Specific Pyrrolopyrimidine Compounds”, and PCT/US2015/024328 titled “MerTK-Specific Pyrazolopyrimidine Compounds”, all filed by Wang, et al. in April 2015 and assigned to the University of North Carolina, describe additional pyrimidines, pyrrolopyrimidines, and pyrazolopyrimidines, respectively, with increased selectivity for MerTK. The therapeutic uses of the MerTK inhibitors disclosed in these applications include uses as anti-infective agents, immunostimulatory and immunomodulatory agents, anti-cancer agents, and as adjunctive agents in combination with chemotherapy, radiation, or other standards of care for neoplasms.

In the patent application PCT/US2015/024381 titled “Therapeutic Uses of Selected Pyrrolopyrimidine Compounds with Anti-Mer Tyrosine Kinase Activity,” data is presented in a melanoma mouse model that shows a survival advantage for either UNC2025 monotherapy or anti-PD1 monotherapy. However, the combination of UNC2025 and anti-PD1 did not provide additional survival benefit beyond that seen with therapeutic doses of monotherapy.

Accordingly, it is an object of the present invention to identify new methods and compositions for the treatment of a tumor, cancer, or other neoplasm.

### SUMMARY OF THE INVENTION

The present invention includes a method of treating a cancer in a host, wherein the cancer is selected from colon cancer, prostate cancer, lung cancer, melanoma, or breast cancer which involves administering an effective amount of UNC2371 in a combination or alternation schedule with an immune checkpoint inhibitor, wherein UNC2371 has the structure:

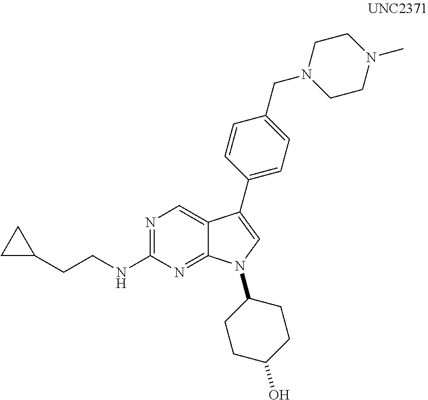

or a pharmaceutically acceptable salt thereof. The MerTK inhibitor UNC2371 was originally disclosed in WO2013/052417 (See Compound 20, pg. 27).

In one embodiment, the immune checkpoint inhibitor is selected from a cytotoxic T-lymphocyte-associated protein 4 (CTLA4) inhibitor, a programmed cell death protein 1 (PD1) inhibitor, a programmed death-ligand 1 (PDL-1) inhibitor, or a combination thereof, and wherein for example the inhibitor can be an antibody such as ipilimumab, nivolumab or pembrolizumab. In one embodiment, UNC2371 and the immune checkpoint inhibitor combination is administered in further combination or alternation with ionizing radiation. In one embodiment, UNC2371 and the immune checkpoint inhibitor combination is administered in further combination or alternation with a Toll-like receptor (TLR) agonist, for example, but not limited to, monophosphoryl lipid A (MPL), Mycobacterium bovis (Bacillus-Calmette Guérin, BCG), CpG, ISCOMatrix, imiquimod (Aldera), Poly IC:LC, OK-432, and/or resiquimod. In one embodiment, UNC2371 and the immune checkpoint inhibitor combination is administered in further combination or alternation with both ionizing radiation and a TLR agonist.

The present invention also includes a method of treating a cancer in a host which involves administering an effective amount of UNC2371 in a combination or alternation schedule with a Toll-like receptor (TLR) agonist and/or radiation.

In another embodiment, a method of treating a cancer in a host is provided that includes administering to the host a therapeutically effective combination of a compound of Formula I and an immune checkpoint inhibitor, wherein the dose administered for either the compound of Formula I or the immune checkpoint inhibitor or both is a subtherapeutic dose for the disorder being treated, wherein Formula I has the structure:

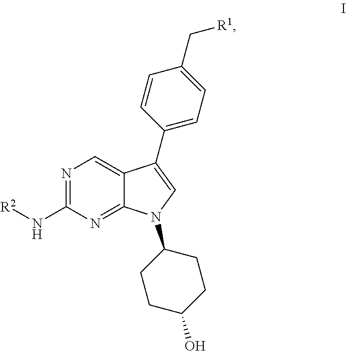

wherein;

R¹ is heterocycle, wherein R¹ is optionally substituted one, two, or three times; and

R² is alkyl, cycloalkyl, or cycloalkylalkyl, wherein R² is optionally substituted one, two, or three times;

or a pharmaceutically acceptable salt thereof.

For example, Formula I can be selected from

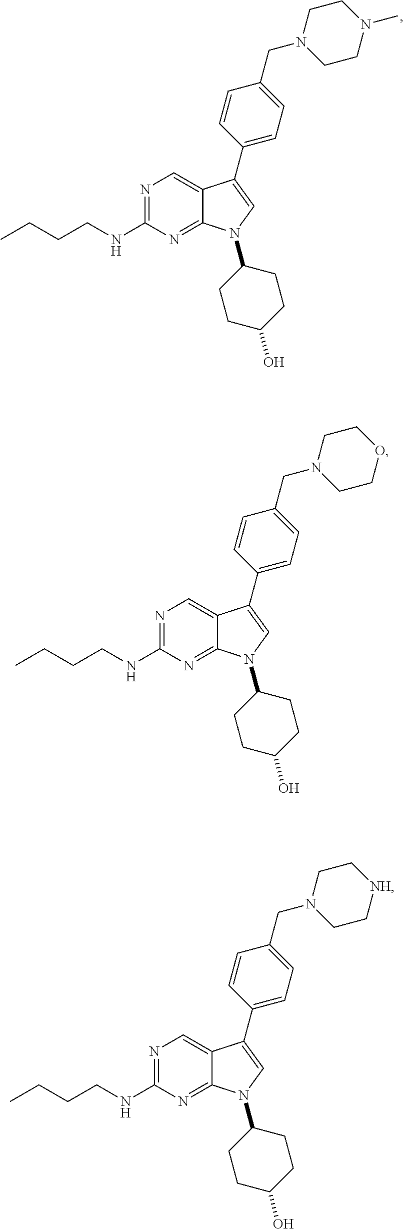

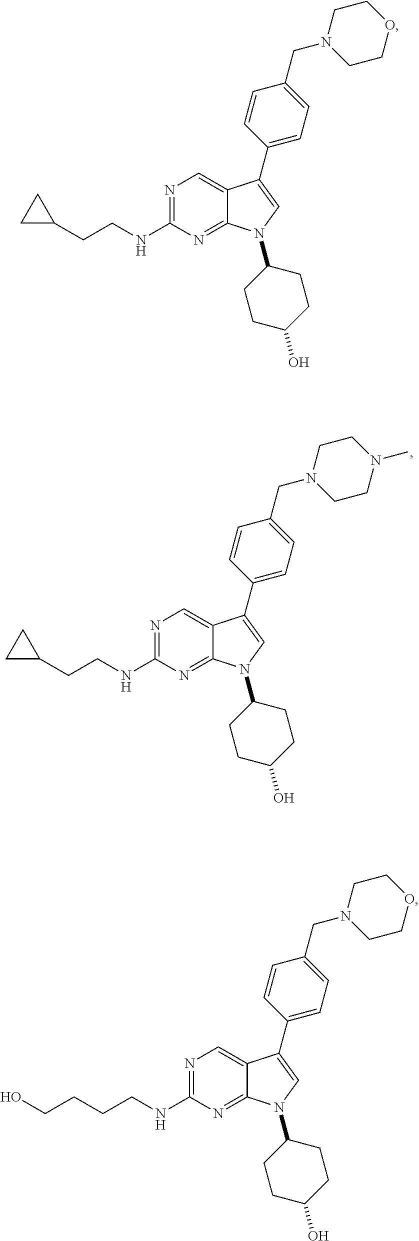

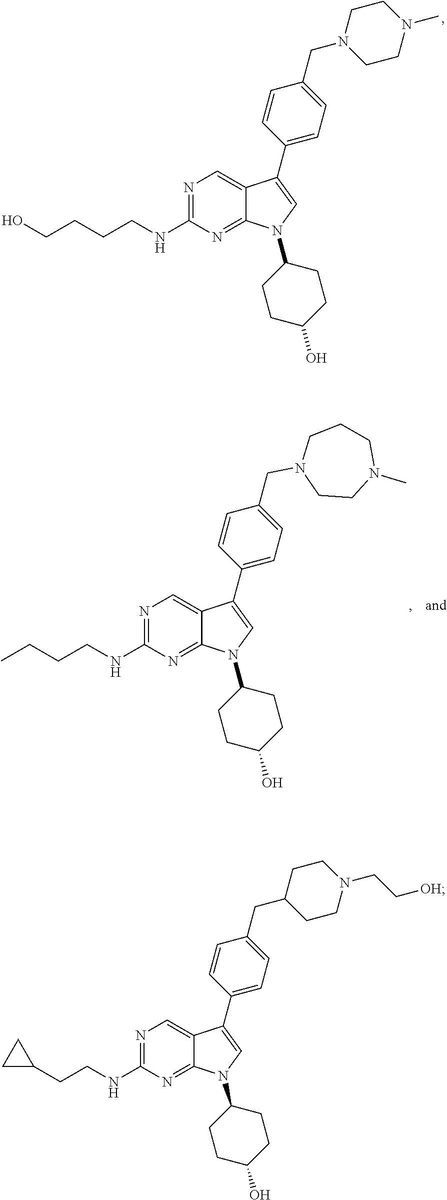

or a pharmaceutically acceptable salt thereof In one embodiment, the MerTK inhibitor and immune checkpoint inhibitor combination is administered in further combination or alternation with ionizing radiation. In one embodiment, the MerTK inhibitor and immune checkpoint inhibitor combination is administered in further combination or alternation with a Toll-like receptor (TLR) agonist. In one embodiment, the MerTK inhibitor and immune checkpoint inhibitor combination is administered in further combination or alternation with both ionizing radiation and a TLR agonist.

In another embodiment, a method of treating a cancer in a host is provided that includes administering to the host a therapeutically effective combination of a compound of Formula I and an immune checkpoint inhibitor, a TLR agonist and/or ionizing radiation, wherein Formula I has the structure:

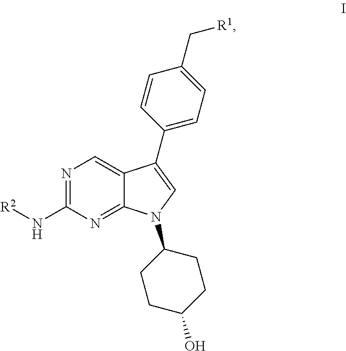

wherein;

R¹ is heterocycle, wherein R¹ is optionally substituted one, two, or three times; and

R² is alkyl, cycloalkyl, or cycloalkylalkyl, wherein R² is optionally substituted one, two, or three times;

or a pharmaceutically acceptable salt thereof.

For example, Formula I can be selected from

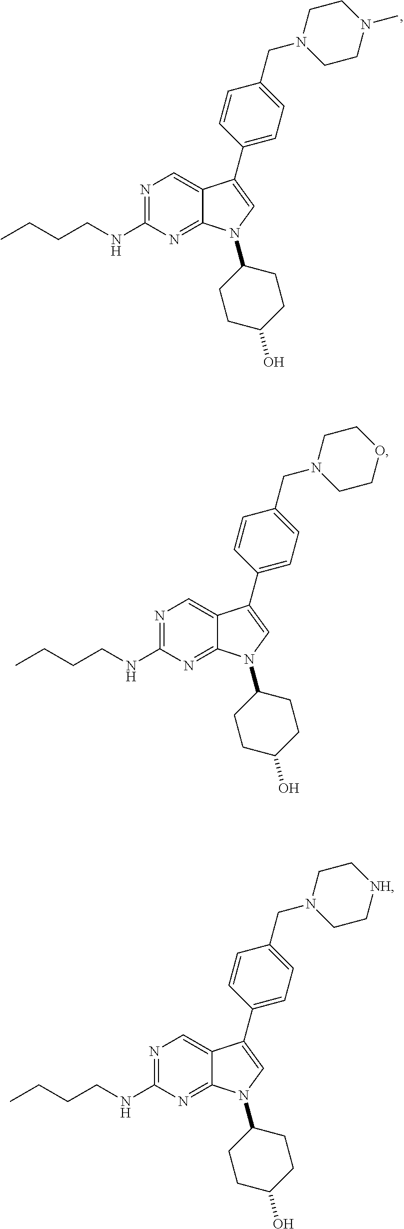

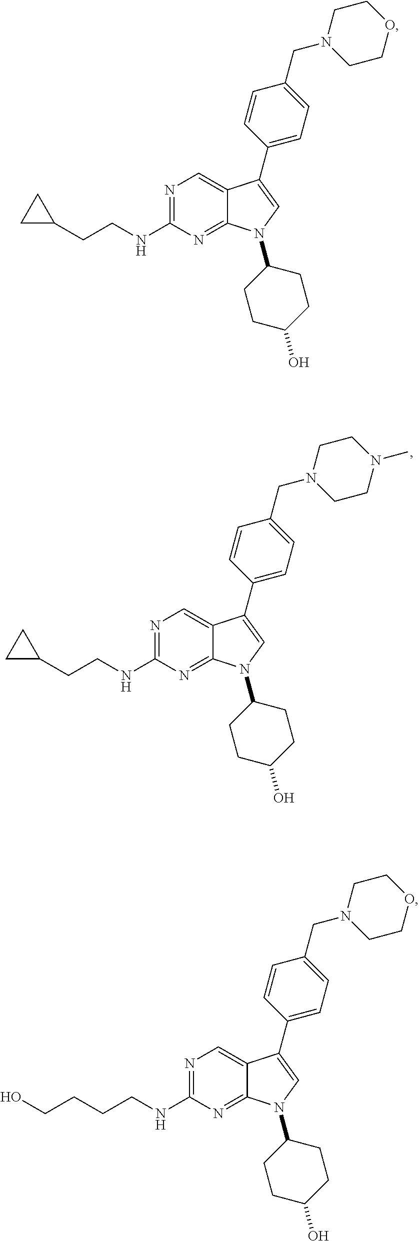

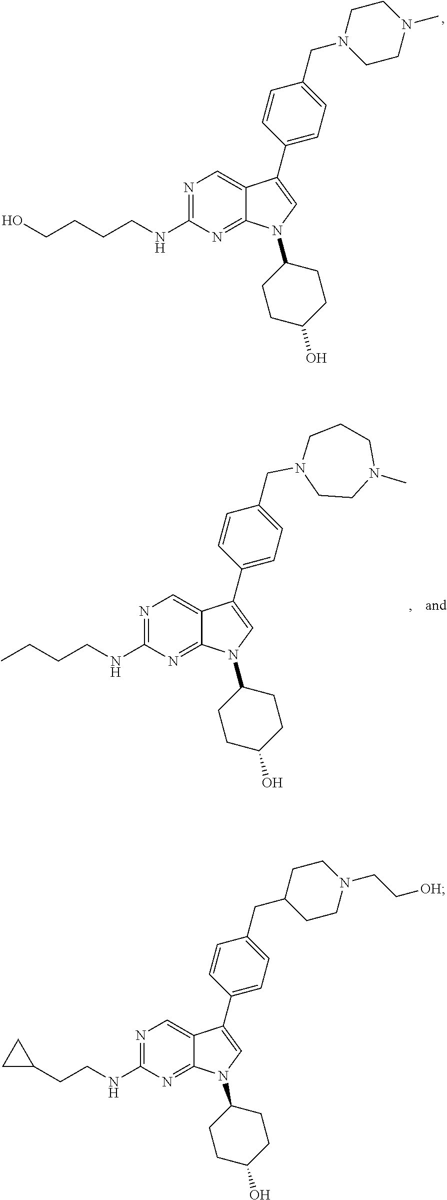

or a pharmaceutically acceptable salt thereof

In yet another embodiment, a method of treating a cancer in a host is provided that includes administering to the host a therapeutically effective combination of a compound of Formula I and an immune checkpoint inhibitor, wherein the cancer is not responsive to immune checkpoint inhibitor monotherapy. In one embodiment, the MerTK inhibitor and immune checkpoint inhibitor combination is administered in further combination or alternation with ionizing radiation. In one embodiment, the MerTK inhibitor and immune checkpoint inhibitor combination is administered in further combination or alternation with a Toll-like receptor (TLR) agonist.

In yet a further embodiment, a method for treating specific cancers comprises administering to a host in need thereof a MerTK inhibitor in combination or alternation with an inhibitor that prevents the downregulation of the immune system (immune checkpoint inhibitor), wherein the administration of the combination results in an additive inhibitory effect in the cancer compared to the use of either the MerTK inhibitor alone or the immune checkpoint inhibitor alone. In one embodiment, the MerTK inhibitor and immune checkpoint inhibitor combination is administered in further combination or alternation with ionizing radiation. In one embodiment, the MerTK inhibitor and immune checkpoint inhibitor combination is administered in further combination or alternation with a Toll-like receptor (TLR) agonist. In one embodiment, the MerTK inhibitor and immune checkpoint inhibitor combination is administered in further combination or alternation with both ionizing radiation and a TLR agonist.

It has been discovered that the use of a MerTK inhibitor as described herein in combination with an immune checkpoint inhibitor that prevents the downregulation of the immune system can inhibit the proliferation and growth of certain cancers, including cancers that otherwise may not be responsive to immune downregulation inhibitor monotherapy, for example certain colon cancers. It has further been demonstrated that these results may be achieved using a dose of a MerTK inhibitor that alone does not significantly inhibit the proliferation or growth of the cancer. That is, this result can be achieved with a subtherapeutic dose of the MerTK inhibitor for the disorder being treated. As shown herein, the use of such a combination can result in the significant and advantageous delay of cancer growth in vivo.

In one aspect of the present invention, a method for treating a specific cancer is provided by administering to a host in need thereof a MerTK inhibitor (for example, UNC2371) in combination or alternation with a cytotoxic T-lymphocyte-associated 4 (CTLA4) immune checkpoint inhibitor, for example, an anti-CTLA-4 antibody, wherein the administration of the combination results in an additive inhibitory effect in the cancer compared to the use of either the MerTK inhibitor alone or the CTLA4 immune checkpoint inhibitor alone. In embodiments of the invention, the host is suffering from a cancer selected from colon cancer, prostate cancer, lung cancer, melanoma, or breast cancer. In one embodiment, the host is suffering from a cancer that otherwise is not responsive to immune checkpoint inhibitor monotherapy, for example, but not limited to, certain colon cancers.

In further aspects of the invention, and without wanting to be bound by any specific theory, it is believed that the combination or alternation of ionizing radiation directed to a tumor further increases its immunicity due to the mechanism by which ionizing radiation kills the tumor cell. In this way, the use of ionizing radiation either before, during, or after MerTK inhibitor and immune checkpoint inhibitor combination therapy may increase the immunogenicity of the tumor allowing for a more robust anti-immunogenic effect in combination with a MerTK inhibitor/immune checkpoint inhibitor combination. In one aspect of the invention, as contemplated herein, the MerTK inhibitor and immune checkpoint inhibitor combination is administered in further combination or alternation with ionizing radiation. In embodiments as contemplated herein, the MerTK inhibitor, immune checkpoint inhibitor, ionizing radiation combination is administered in further combination or alternation with a Toll-like receptor (TLR) agonist.

In further aspects of the invention, and without wanting to be bound by any specific theory, it is believed that the use of a TLR receptor agonist further increases the immunological effect directed towards the cancer cell, providing for a more robust anti-immunogenic effect in combination with a MerTK inhibitor. In embodiments, the MerTk inhibitors described herein are administered in combination or alternation with a TRL agonist. In certain embodiments, the MerTk inhibitor and TRL agonist is administered in combination or alternation with an immune checkpoint inhibitor and/or ionizing radition.

As contemplated herein, the MerTK inhibitor and immune checkpoint inhibitor can be administered in temporal combination or temporal alternation. For example, the two agents can be administered together or independently, for example by different routes such as, but not limited to, oral administration, intravenous administration, and injection. As described further below, the MerTK inhibitor and immune checkpoint inhibitor are administered such that the effect of the two agents overlap in vivo to create the advantageous effect. Likewise, the two active agents can be administered in temporal alternation instead of temporal combination (regardless of physical form of administration) as long as the effect of the two agents overlap in vivo to create the advantageous effect. Likewise, the MerTK inhibitor, immune checkpoint inhibitor, TLR agonist and/or ionizing radiation can be administered in temporal combination or temporal alternation.

As part of the invention, one or more of the compounds of Formula I, in combination with an immune checkpoint inhibitor, can be used as adjunctive antineoplastic therapy for its immunostimulatory effect as a means to increase the efficacy of the antineoplastic standard of care therapies, such as chemotherapeutic compounds or ionizing radiation.

In summary, the present invention includes the following features:

A) Methods for treating a host suffering from a cancer comprising administering the MerTK inhibitor UNC2371 in physical or temporal combination with an immune checkpoint inhibitor, for example an antibody;

B) Methods for treating a host suffering from a cancer comprising administering the MerTK inhibitor UNC2371 in physical or temporal combination with an immune checkpoint inhibitor, for example an antibody, and in further combination with a TLR agonist and/or ionizing radiation;

C) Methods for treating a host suffering from a cancer comprising administering the MerTK inhibitor UNC2371 in physical or temporal combination with an immune checkpoint inhibitor, for example an antibody, and the cancer is selected from colon, prostate, lung, melanoma, and breast cancer;

D) Methods for treating a host suffering from a cancer comprising administering the MerTK inhibitor UNC2371 in physical or temporal combination with an immune checkpoint inhibitor, for example an antibody, and the cancer is selected from colon, prostate, lung, melanoma, and breast cancer, and in further combination with a TLR agonist and/or ionizing radiation;

E) Methods for treating a host suffering from a cancer comprising administering the MerTK inhibitor UNC2371 in physical or temporal combination with an immune checkpoint inhibitor, for example an antibody, and the cancer is colon cancer;

F) Methods for treating a host suffering from a cancer comprising administering the MerTK inhibitor UNC2371 in physical or temporal combination with an immune checkpoint inhibitor, for example an antibody, and the cancer is colon cancer, and in further combination with a TLR agonist and/or ionizing radiation;

G) Methods for treating a host suffering from a cancer comprising administering the MerTK inhibitor UNC2371 in physical or temporal combination with an immune checkpoint inhibitor, for example an antibody, wherein the MerTK inhibitor is administered at a subtherapeutic dose for the disorder being treated;

H) Methods for treating a host suffering from a cancer comprising administering the MerTK inhibitor UNC2371 in physical or temporal combination with an immune checkpoint inhibitor, for example an antibody, wherein the MerTK inhibitor is administered at a subtherapeutic dose for the disorder being treated, and in further combination with a TLR agonist and/or ionizing radiation;

I) Methods for treating a host suffering from a cancer comprising administering the MerTK inhibitor UNC2371 in physical or temporal combination with a CTLA4 immune checkpoint inhibitor, for example an antibody;

J) Methods for treating a host suffering from a cancer comprising administering the MerTK inhibitor UNC2371 in physical or temporal combination with a CTLA4 immune checkpoint inhibitor, for example an antibody, and in further combination with a TLR agonist and/or ionizing radiation;

K) Methods for treating a host suffering from a cancer comprising administering the MerTK inhibitor UNC2371 in physical or temporal combination with a CTLA4 immune checkpoint inhibitor, for example an antibody;

L) Methods for treating a host suffering from a cancer comprising administering the MerTK inhibitor UNC2371 in physical or temporal combination with a CTLA4 immune checkpoint inhibitor, for example an antibody, and in further combination with a TLR agonist and/or ionizing radiation;

M) Methods for treating a host suffering from a cancer comprising administering the MerTK inhibitor UNC2371 in physical or temporal combination with a CTLA4 immune checkpoint inhibitor, for example an antibody and the cancer is selected from colon, prostate, lung, melanoma, and breast cancer;

N) Methods for treating a host suffering from a cancer comprising administering the MerTK inhibitor UNC2371 in physical or temporal combination with a CTLA4 immune checkpoint inhibitor, for example an antibody and the cancer is selected from colon, prostate, lung, melanoma, and breast cancer, and in further combination with a TLR agonist and/or ionizing radiation;

O) Methods for treating a host suffering from a cancer comprising administering the MerTK inhibitor UNC2371 in physical or temporal combination with a CTLA4 immune checkpoint inhibitor, for example an antibody, and the cancer is colon cancer;

P) Methods for treating a host suffering from a cancer comprising administering the MerTK inhibitor UNC2371 in physical or temporal combination with a CTLA4 immune checkpoint inhibitor, for example an antibody, and the cancer is colon cancer, and in further combination with a TLR agonist and/or ionizing radiation;

Q) Methods for treating a host suffering from a cancer comprising administering the MerTK inhibitor UNC2371 in physical or temporal combination with a CTLA4 immune checkpoint inhibitor, for example an antibody, wherein the MerTK inhibitor is administered at a subtherapeutic dose for the disorder being treated;

R) Methods for treating a host suffering from a cancer comprising administering the MerTK inhibitor UNC2371 in physical or temporal combination with a CTLA4 immune checkpoint inhibitor, for example an antibody, wherein the MerTK inhibitor is administered at a subtherapeutic dose for the disorder being treated, and in further combination with a TLR agonist and/or ionizing radiation;

S) Methods for treating a host suffering from a cancer comprising administering a MerTK inhibitor of Formula I in physical or temporal combination with an immune checkpoint inhibitor, for example an antibody, wherein the immune checkpoint inhibitor is administered at a subtherapeutic dose for the disorder being treated;

T) Methods for treating a host suffering from a cancer comprising administering a MerTK inhibitor of Formula I in physical or temporal combination with an immune checkpoint inhibitor, for example an antibody, wherein the immune checkpoint inhibitor is administered at a subtherapeutic dose for the disorder being treated, and in further combination with a TLR agonist and/or ionizing radiation;

U) Methods for treating a host suffering from a cancer comprising administering a MerTK inhibitor of Formula I in physical or temporal combination with an immune checkpoint inhibitor, for example an antibody, wherein the MerTK inhibitor is administered at a subtherapeutic dose for the disorder being treated, and the cancer is selected from colon, prostate, lung, melanoma, and breast cancer;

V) Methods for treating a host suffering from a cancer comprising administering a MerTK inhibitor of Formula I in physical or temporal combination with an immune checkpoint inhibitor, for example an antibody, wherein the MerTK inhibitor is administered at a subtherapeutic dose for the disorder being treated, and the cancer is selected from colon, prostate, lung, melanoma, and breast cancer, and in further combination with a TLR agonist and/or ionizing radiation;

W) Methods for treating a host suffering from a cancer comprising administering a MerTK inhibitor of Formula I in combination with a CTLA4 immune checkpoint inhibitor, for example an antibody, wherein the MerTK inhibitor is administered at a subtherapeutic dose for the disorder treated, and the cancer is selected from colon, prostate, lung, melanoma, and breast cancer;

X) Methods for treating a host suffering from a cancer comprising administering a MerTK inhibitor of Formula I in combination with a CTLA4 immune checkpoint inhibitor, for example an antibody, wherein the MerTK inhibitor is administered at a subtherapeutic dose for the disorder treated, and the cancer is selected from colon, prostate, lung, melanoma, and breast cancer, and in further combination with a TLR agonist and/or ionizing radiation;

Y) Methods for treating a host suffering from a cancer comprising administering a MerTK inhibitor of Table 1 in combination with an immune checkpoint inhibitor, for example an antibody, wherein the immune checkpoint inhibitor is administered at a subtherapeutic dose for the disorder treated;

Z) Methods for treating a host suffering from a cancer comprising administering a MerTK inhibitor of Table 1 in combination with an immune checkpoint inhibitor, for example an antibody, wherein the immune checkpoint inhibitor is administered at a subtherapeutic dose for the disorder treated, and in further combination with a TLR agonist and/or ionizing radiation;

AA) Methods for treating a host suffering from a cancer comprising administering a MerTK inhibitor of Table 1 in combination with an immune checkpoint inhibitor, for example an antibody, wherein the MerTK inhibitor is administered at a subtherapeutic dose for the disorder treated, and the cancer is selected from colon, prostate, lung, melanoma, and breast cancer;

BB) Methods for treating a host suffering from a cancer comprising administering a MerTK inhibitor of Table 1 in combination with an immune checkpoint inhibitor, for example an antibody, wherein the MerTK inhibitor is administered at a subtherapeutic dose for the disorder treated, and the cancer is selected from colon, prostate, lung, melanoma, and breast cancer, and in further combination with a TLR agonist and/or ionizing radiation;

CC) Methods for treating a host suffering from a cancer comprising administering a MerTK inhibitor of Table 1 in combination with a CTLA4 immune checkpoint inhibitor, for example an antibody, wherein the MerTK inhibitor is administered at a subtherapeutic dose for the disorder treated, and the cancer is selected from colon, prostate, lung, melanoma, and breast cancer;

DD) Methods for treating a host suffering from a cancer comprising administering a MerTK inhibitor of Table 1 in combination with a CTLA4 immune checkpoint inhibitor, for example an antibody, wherein the MerTK inhibitor is administered at a subtherapeutic dose for the disorder treated, and the cancer is selected from colon, prostate, lung, melanoma, and breast cancer, and in further combination with a TLR agonist and/or ionizing radiation;

EE) A pharmaceutically acceptable composition for use as a therapeutic comprising a compound of Formula I, or a salt, isotopic analog, prodrug, or a combination thereof, and an immune checkpoint inhibitor, for example an antibody, including where one or both is provided at a subtherapeutic dosage for the disorder being treated;

FF) Use of a compound of Formula I, or a pharmaceutically acceptable composition, salt, isotopic analog, or prodrug thereof, in combination with an immune checkpoint inhibitor, for example an antibody, in the manufacture of a medicament for use as a therapeutic to treat a host with cancer, including where one or both is provided at a subtherapeutic dosage for the disorder being treated;

GG) Processes for the preparation of therapeutic products that contain an effective amount of a compound of Formula I in combination with an immune checkpoint inhibitor, for example an antibody, for use in the treatment of a host having cancer, including where one or both is provided at a subtherapeutic dosage for the disorder being treated; and

HH) A method for manufacturing a medicament selected from a compound of Formula I in combination with an immune checkpoint inhibitor, including an antibody, intended for therapeutic use as a chemotherapeutic for the treatment of a cancer, for example where one or both is provided at a subtherapeutic dosage for the disorder being treated.

### BRIEF DESCRIPTION OF FIGURES

FIG. 1 illustrates the median tumor volumes (mm³) vs. time (days) in mice treated with placebo, UNC2371, anti-PD1, anti-CTLA4, UNC2371 and anti-PD1, or UNC2371 and anti-CTLA4. Mice were treated with PBS; UNC2371 (1.34 mg/kg, po, tid×21 first day 2 doses); UNC2371 (6.72 mg/kg, po, tid×21 first day 2 doses); UNC2371 (20.16 mg/kg, po, tid×21 first day 2 doses); anti-PD1 RMP1-14 (5 mg/kg, ip, biwk×2); anti-CTLA4 9H10 (5 mg/kg, ip, day 1) followed by anti-CTLA4 9H10 (2.5 mg/kg, ip, days 4, 7); UNC2371 (1.34 mg/kg, po, tid×21 first day 2 doses) and anti-PD1 RMP1-14 (5 mg/kg, ip, biwk×2); UNC2371 (6.72 mg/kg, po, tid×21 first day 2 doses) and anti-PD1 RMP1-14 (5 mg/kg, ip, biwk×2); UNC2371 (20.16 mg/kg, po, tid×21 first day 2 doses) and anti-PD1 RMP1-14 (5 mg/kg, ip, biwk×2); UNC2371 (1.34 mg/kg, po, tid×21 first day 2 doses) anti-CTLA4 9H10 (5 mg/kg, ip, day 1) followed by anti-CTLA4 9H10 (2.5 mg/kg, ip, days 4, 7); UNC2371 (6.72 mg/kg, po, tid×21 first day 2 doses) anti-CTLA4 9H10 (5 mg/kg, ip, day 1) followed by anti-CTLA4 9H10 (2.5 mg/kg, ip, days 4, 7); or UNC2371 (20.16 mg/kg, po, tid×21 first day 2 doses) and anti-CTLA4 9H10 (5 mg/kg, ip, day 1) followed by anti-CTLA4 9H10 (2.5 mg/kg, ip, days 4, 7).

FIG. 2 illustrates the mean tumor volumes (mm³) vs. time (days) in mice treated with placebo, UNC2371, anti-PD1, anti-CTLA4, UNC2371 and anti-PD1, or UNC2371 and anti-CTLA4. Mice were treated with PBS; UNC2371 (1.34 mg/kg, po, tid×21 first day 2 doses); UNC2371 (6.72 mg/kg, po, tid×21 first day 2 doses); UNC2371 (20.16 mg/kg, po, tid×21 first day 2 doses); anti-PD1 RMP1-14 (5 mg/kg, ip, biwk×2); anti-CTLA4 9H10 (5 mg/kg, ip, day 1) followed by anti-CTLA4 9H10 (2.5 mg/kg, ip, days 4, 7); UNC2371 (1.34 mg/kg, po, tid×21 first day 2 doses) and anti-PD1 RMP1-14 (5 mg/kg, ip, biwk×2); UNC2371 (6.72 mg/kg, po, tid×21 first day 2 doses) and anti-PD1 RMP1-14 (5 mg/kg, ip, biwk×2); UNC2371 (20.16 mg/kg, po, tid×21 first day 2 doses) and anti-PD1 RMP1-14 (5 mg/kg, ip, biwk×2); UNC2371 (1.34 mg/kg, po, tid×21 first day 2 doses) anti-CTLA4 9H10 (5 mg/kg, ip, day 1) followed by anti-CTLA4 9H10 (2.5 mg/kg, ip, days 4, 7); UNC2371 (6.72 mg/kg, po, tid×21 first day 2 doses) anti-CTLA4 9H10 (5 mg/kg, ip, day 1) followed by anti-CTLA4 9H10 (2.5 mg/kg, ip, days 4, 7); or UNC2371 (20.16 mg/kg, po, tid×21 first day 2 doses) and anti-CTLA4 9H10 (5 mg/kg, ip, day 1) followed by anti-CTLA4 9H10 (2.5 mg/kg, ip, days 4, 7).

FIG. 3 illustrates the survival (percent remaining) vs. time (days) in mice treated with placebo, UNC2371, anti-PD1 antibody, anti-CTLA4 antibody, UNC2371 and anti-PD1 antibody, or UNC2371 and anti-CTLA4 antibody. Mice were treated with PBS; UNC2371 (1.34 mg/kg, po, tid×21 first day 2 doses); UNC2371 (6.72 mg/kg, po, tid×21 first day 2 doses); UNC2371 (20.16 mg/kg, po, tid×21 first day 2 doses); anti-PD1 RMP1-14 (5 mg/kg, ip, biwk×2); anti-CTLA4 9H10 (5 mg/kg, ip, day 1) followed by anti-CTLA4 9H10 (2.5 mg/kg, ip, days 4, 7); UNC2371 (1.34 mg/kg, po, tid×21 first day 2 doses) and anti-PD1 RMP1-14 (5 mg/kg, ip, biwk×2); UNC2371 (6.72 mg/kg, po, tid×21 first day 2 doses) and anti-PD1 RMP1-14 (5 mg/kg, ip, biwk×2); UNC2371 (20.16 mg/kg, po, tid×21 first day 2 doses) and anti-PD1 RMP1-14 (5 mg/kg, ip, biwk×2); UNC2371 (1.34 mg/kg, po, tid×21 first day 2 doses) anti-CTLA4 9H10 (5 mg/kg, ip, day 1) followed by anti-CTLA4 9H10 (2.5 mg/kg, ip, days 4, 7); UNC2371 (6.72 mg/kg, po, tid×21 first day 2 doses) anti-CTLA4 9H10 (5 mg/kg, ip, day 1) followed by anti-CTLA4 9H10 (2.5 mg/kg, ip, days 4, 7); or UNC2371 (20.16 mg/kg, po, tid×21 first day 2 doses) and anti-CTLA4 91110 (5 mg/kg, ip, day 1) followed by anti-CTLA4 9H10 (2.5 mg/kg, ip, days 4, 7).

FIG. 4 is a graph of tumor volume (mm³) vs. time (days) in mice treated with placebo. Each line represents an individual mouse and tumor volumes were measured two times per week.

FIG. 5 is a graph of tumor volume (mm³) vs. time (days) in mice treated with 3 mg/kg UNC2371. Each line represents an individual mouse and tumor volumes were measured two times per week.

FIG. 6 is a graph of tumor volume (mm³) vs. time (days) in mice treated with 15 mg/kg UNC2371. Each line represents an individual mouse and tumor volumes were measured two times per week.

FIG. 7 is a graph of tumor volume (mm³) vs. time (days) in mice treated with 45 mg/kg UNC2371. Each line represents an individual mouse and tumor volumes were measured two times per week.

FIG. 8 is a graph of tumor volume (mm³) vs. time (days) in mice treated with anti-PD1 antibody RMP1-14 (5 mg/kg, ip, biwk×2). Each line represents an individual mouse and tumor volumes were measured two times per week.

FIG. 9 is a graph of tumor volume (mm³) vs. time (days) in mice treated with 3 mg/kg UNC2371 and anti-PD1 antibody RMP1-14 (5 mg/kg, ip, biwk×2). Each line represents an individual mouse and tumor volumes were measured two times per week.

FIG. 10 is a graph of tumor volume (mm³) vs. time (days) in mice treated with 15 mg/kg UNC2371 and anti-PD1 antibody RMP1-14 (5 mg/kg, ip, biwk×2). Each line represents an individual mouse and tumor volumes were measured two times per week.

FIG. 11 is a graph of tumor volume (mm³) vs. time (days) in mice treated with 45 mg/kg UNC2371 and anti-PD1 antibody RMP1-14 (5 mg/kg, ip, biwk×2). Each line represents an individual mouse and tumor volumes were measured two times per week.

FIG. 12 is a graph of tumor volume (mm³) vs. time (days) in mice treated with anti-CTLA4 antibody 9H10 (5 mg/kg, ip, day 1; 2.5 mg/kg, ip, days 4, 7). Each line represents an individual mouse and tumor volumes were measured two times per week.

FIG. 13 is a graph of tumor volume (mm³) vs. time (days) in mice treated with 3 mg/kg UNC2371 and anti-CTLA4 antibody 9H10 (5 mg/kg, ip, day 1; 2.5 mg/kg, ip, days 4, 7). Each line represents an individual mouse and tumor volumes were measured two times per week.

FIG. 14 is a graph of tumor volume (mm³) vs. time (days) in mice treated with 15 mg/kg UNC2371 and anti-CTLA4 antibody 9H10 (5 mg/kg, ip, day 1; 2.5 mg/kg, ip, days 4, 7). Each line represents an individual mouse and tumor volumes were measured two times per week.

FIG. 15 is a graph of tumor volume (mm³) vs. time (days) in mice treated with 45 mg/kg UNC2371 and anti-CTLA4 antibody 9H10 (5 mg/kg, ip, day 1; 2.5 mg/kg, ip, days 4, 7). Each line represents an individual mouse and tumor volumes were measured two times per week.

FIG. 16 is a graph showing the tumor volume (mm³) vs. time after UNC2371 treatment initiation (days) in a mouse model of colon carcinoma (Colon26 syngeneic model). Mice were treated in groups (N=10) according to the following: deionized water via oral gavage (p.o.) three times daily for twenty-one days, starting with two doses on Day 1 (tid×21 first day two doses) and PBS i.p. twice weekly for two weeks (biwk×2) (PBO; diamonds); 1 mg/kg UNC2371 via oral gavage (p.o.) three times daily for twenty-one days, starting with two doses on Day 1 (tid×21 first day two doses) (LD-UNC2371; squares); 5 mg/kg UNC2371 via oral gavage (p.o.) three times daily for twenty-one days, starting with two doses on Day 1 (tid×21 first day two doses) (MD-UNC2371; triangles); 15 mg/kg UNC2371 via oral gavage (p.o.) three times daily for twenty-one days, starting with two doses on Day 1 (tid×21 first day two doses) (HD-UNC2371; X markers). As discussed in Example 3, the median time to endpoint (TTE) for low dose (1 mg/kg TID), mid dose (5 mg/kg TID) and high dose (15 mg/kg TID) UNC2371 were 14.0, 16.7, and 20.7 days, respectively, corresponding to tumor growth delay of 0.2 days (1%), 2.9 days (21%), and 6.9 days (50%).

FIG. 17 is a graph showing the tumor volume (mm³) vs. time after anti-CTLA4 or anti-CTLA4/UNC2371 treatment initiation (days) in a mouse model of colon carcinoma (Colon26 syngeneic model). Mice were treated in groups (N=10) according to the following: deionized water via oral gavage (p.o.) three times daily for twenty-one days, starting with two doses on Day 1 (tid×21 first day two doses) and PBS i.p. twice weekly for two weeks (biwk×2) (PBO; diamonds); anti-CTLA4 mAb at a dosage of 5 mg/kg on Day 1 and at 2.5 mg/kg on Days 4 and 7 (antiCTLA4; squares); 1 mg/kg UNC2371 via oral gavage (p.o.) three times daily for twenty-one days, starting with two doses on Day 1 (tid×21 first day two doses) and anti-CTLA4 mAb at a dosage of 5 mg/kg on Day 1 and at 2.5 mg/kg on Days 4 and 7 (LD-UNC2371+aPD1; triangles); 5 mg/kg UNC2371 via oral gavage (p.o.) three times daily for twenty-one days, starting with two doses on Day 1 (tid×21 first day two doses) and anti-CTLA4 mAb at a dosage of 5 mg/kg on Day 1 and at 2.5 mg/kg on Days 4 and 7 (MD-UNC2371+aPD1; X markers); 15 mg/kg UNC2371 via oral gavage (p.o.) three times daily for twenty-one days, starting with two doses on Day 1 (tid×21 first day two doses) and anti-CTLA4 mAb at a dosage of 5 mg/kg on Day 1 and at 2.5 mg/kg on Days 4 and 7 (HD-UNC2371+aPD1; asterisks). As discussed in Example 3, the median time to endpoint (TTE) for low dose, mid dose, and high dose UNC2371 in combination with anti-CTLA4 was 27.1, 20.7, or 22.3 days, respectively, corresponding to tumor growth delay of 13.3 days (96%), 6.9 days (50%) and 8.5 days (62%).

FIG. 18 is a graph showing the tumor volume (mm³) vs. time after anti-PD1 or anti-PD1/UNC2371 treatment initiation (days) in a mouse model of colon carcinoma (Colon26 syngeneic model). Mice were treated in groups (N=10) according to the following: deionized water via oral gavage (p.o.) three times daily for twenty-one days, starting with two doses on Day 1 (tid×21 first day two doses) and PBS i.p. twice weekly for two weeks (biwk×2) (PBO; diamonds); anti-PD1 mAb at a dosage of 5 mg/kg i.p. biwk×2 (aPD-1; squares); 1 mg/kg UNC2371 via oral gavage (p.o.) three times daily for twenty-one days, starting with two doses on Day 1 (tid×21 first day two doses) and anti-PD1 mAb at a dosage of 5 mg/kg i.p. biwk×2 (LD-UNC2371+aPD1; triangles); 5 mg/kg UNC2371 via oral gavage (p.o.) three times daily for twenty-one days, starting with two doses on Day 1 (tid×21 first day two doses) and anti-PD1 mAb at a dosage of 5 mg/kg i.p. biwk×2 (MD-UNC2371+aPD1; X markers); 15 mg/kg UNC2371 via oral gavage (p.o.) three times daily for twenty-one days, starting with two doses on Day 1 (tid×21 first day two doses) and anti-PD1 mAb at a dosage of 5 mg/kg i.p. biwk×2 (HD-UNC2371+aPD1; aterisks). As discussed in Example 3, the median time to endpoint (TTE) for low dose, mid dose, and high dose UNC2371 in combination with anti-PD1 was 14.5, 15.8, or 17.7 days, respectively, corresponding to tumor growth delay of 0.7 days (5%), 2.0 days (14%) and 3.9 days (28%).

FIG. 19 is a graph showing the number of cells in the tumor (relative fluorescence intensity) for CD4+ (dark bars) or CD8+(light gray bars) positive T-cells in a mouse model of colon carcinoma (Colon26 syngeneic model). Mice were treated according to the figure legends for FIGS. 16 to 18 for the following twelve treatment groups: control (PBO), low dose UNC2371, mid dose UNC2371, high dose UNC2371, anti-PD1, anti-CTLA4, low dose UNC2371+anti-PD1, mid dose UNC2371+anti-PD1, high dose UNC2371 +anti-PD1, low dose UNC2371+anti-CTLA4, mid dose UNC2371+anti-CTLA4, or high dose UNC2371+anti-CTLA4. As discussed in Example 4, UNC2371 treatment was associated with an increase in CD8+ cells in the tumor compared to placebo. Anti-CTLA-4 treatment was associated with the largest CD8+ (cytotoxic lymphocytes) increase, and the combination of UNC2371 (low dose or medium dose) and anti-CTLA4 showed an increase in CD8+ lymphocytes similar to anti-CTLA4 monotherapy.

FIG. 20 is a graph showing the number of cells in the tumor (relative fluorescence intensity) for natural killer (NK) (dark bars) or myeloid-derived suppressor cells (MDSC) (light gray bars) in a mouse model of colon carcinoma (Colon26 syngeneic model). Mice were treated according to the figure legends for FIGS. 16 to 18 for the following twelve treatment groups: control (PBO), low dose UNC2371, mid dose UNC2371, high dose UNC2371, anti-PD1, anti-CTLA4, low dose UNC2371+anti-PD1, mid dose UNC2371+anti-PD1, high dose UNC2371+anti-PD1, low dose UNC2371+anti-CTLA4, mid dose UNC2371+anti-CTLA4, or high dose UNC2371+anti-CTLA4. As discussed in Example 4, there was an increase in Natural Killer (NK) cells with UNC2371 solo therapy as well as UNC2371/anti-CTLA4 combination treatment. In contrast, anti-CTLA4 treatment alone was associated with a decrease in NK cells in the tumor at Day 8.

FIG. 21 is a graph showing the percentage of natural killer cells (panNK marker) (% relative to live cells) from the peripheral blood for a mouse model of colon carcinoma (Colon26 syngeneic model). Mice were treated according to the figure legends for FIGS. 16 to 18 for the following twelve treatment groups: control (PBO; Group 1), low dose UNC2371 (Group 2), mid dose UNC2371 (Group 3), high dose UNC2371 (Group 4), anti-PD1 (Group 5), anti-CTLA4 (Group 6), low dose UNC2371+anti-PD1 (Group 7), mid dose UNC2371+anti-PD1 (Group 8), high dose UNC2371+anti-PD1 (Group 9), low dose UNC2371+anti-CTLA4 (Group 10), mid dose UNC2371+anti-CTLA4 (Group 11), or high dose UNC2371+anti-CTLA4 (Group 12). As discussed in Example 4, the combination of a high dose of UNC2371 with anti-CTLA4 provided the largest increase in the percentage of natural killer (NK) immune cells.

FIG. 22A is a graph showing secondary tumor volume (mm³) vs. time (days) for mice (B16 melanoma model) treated with radiation therapy (XRT) (triangles), UNC2371 (squares), anti-PD1 (circles), or anti-PD1+UNC2371 (diamonds). As discussed in Example 5, all treatment arms were more effective than XRT alone.

FIG. 22B is a graph showing secondary tumor volume (mm³) vs. time (days) for mice (B16 melanoma model) treated with radiation therapy (XRT) (circles), XRT+UNC2371 (upside down triangles), XRT+anti-PD1 (squares), or XRT+anti-PD1 plus UNC2371 (triangles). As discussed in Example 5, all treatment arms enhanced the abscopal effect on the secondary tumor.

FIG. 22C is a graph showing tumor derived monocyte cells (%) for mice (B16 melanoma model) treated with radiation therapy (XRT) or XRT+UNC2371. As discussed in Example 5, there was a decrease in tumor derived myeloid-derived suppressor cells (MDSCs) in mice treated with UNC2371.

FIG. 23A is a graph showing iNOS mRNA expression levels in mice (B16 melanoma model) treated with vehicle, 10 mg/kg UNC2371, 25 mg/kg UNC2371, or 50 mg/kg UNC2371. As discussed in Example 5, there was a decrease in iNOS (inducible nitric oxide synthase) mRNA expression in mice treated with 10, 25, or 50 mg/kg UNC2371 as compared to vehicle control.

FIG. 23B is a graph showing Arginase mRNA expression levels in mice (B16 melanoma model) treated with vehicle, 10 mg/kg UNC2371, 25 mg/kg UNC2371, or 50 mg/kg UNC2371. As discussed in Example 5, there was a decrease in Arginase mRNA expression in mice treated with 25 mg/kg UNC2371 as compared to vehicle control.

FIG. 23C is a graph showing IDO mRNA expression levels in mice (B16 melanoma model) treated with vehicle, 10 mg/kg UNC2371, 25 mg/kg UNC2371, or 50 mg/kg UNC2371. As discussed in Example 5, there was a decrease in IDO (indoleamine 2,3-dioxygenase) mRNA expression in mice treated with 10 or 25 mg/kg UNC2371 as compared to vehicle control.

### DETAILED DESCRIPTION OF THE INVENTION

The present invention includes a method of treating a cancer in a host, wherein the cancer is selected from colon cancer, prostate cancer, lung cancer, melanoma, or breast cancer which involves administering an effective amount of UNC2371 in a combination or alternation schedule with an immune checkpoint inhibitor, for example an antibody, wherein UNC2371 has the structure:

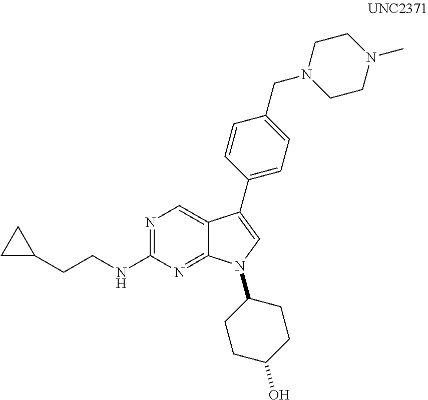

or a pharmaceutically acceptable salt thereof. The MerTK inhibitor UNC2371 was originally disclosed in WO2013/052417 (See Compound 20, pg. 27).

In one embodiment, the immune checkpoint inhibitor is selected from a cytotoxic T-lymphocyte-associated protein 4 (CTLA4) inhibitor, a programmed cell death protein 1 (PD1) inhibitor, or a programmed death-ligand 1 (PDL-1) inhibitor, and wherein the inhibitor can be an antibody, for example, ipilimumab, nivolumab or pembrolizumab.

In another embodiment, a method of treating a cancer in a host is provided that includes administering to the host a therapeutically effective combination of a compound of Formula I and an immune checkpoint inhibitor, wherein the dose administered for either the compound of Formula I or the immune checkpoint inhibitor or both is a subtherapeutic dose for the disorder being treated, wherein Formula I has the structure:

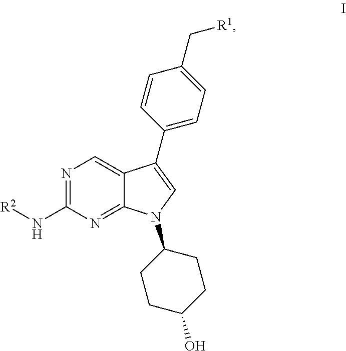

wherein;

R¹ is heterocycle, wherein R¹ is optionally substituted one, two, or three times; and

R² is alkyl, cycloalkyl, or cycloalkylalkyl, wherein R² is optionally substituted one, two, or three times;

or a pharmaceutically acceptable salt thereof.

For example, Formula I can be selected from

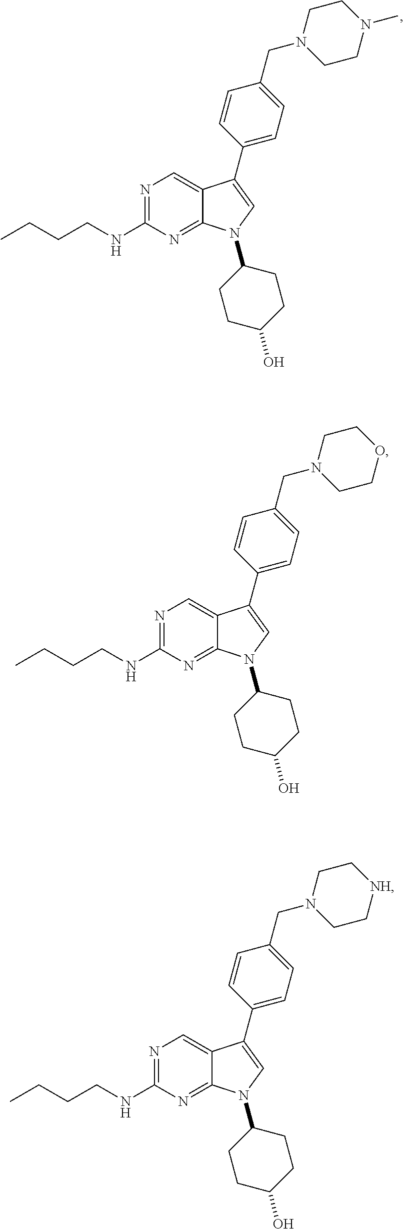

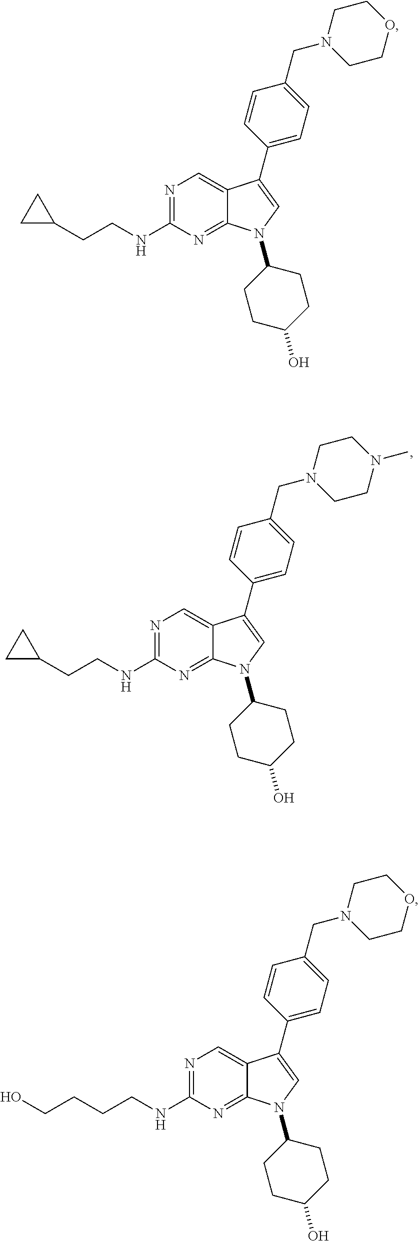

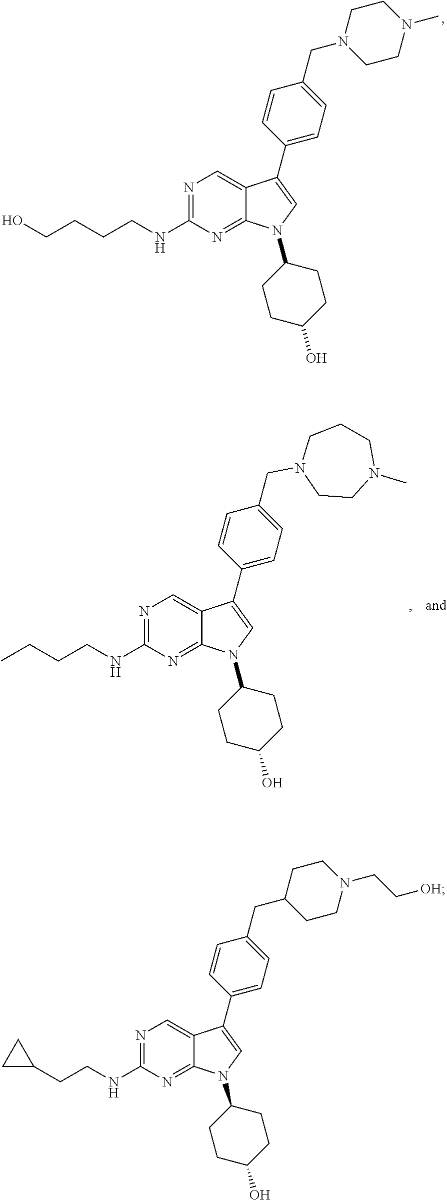

or a pharmaceutically acceptable salt thereof

In yet another embodiment, method of treating a cancer in a host is provided that includes administering to the host a therapeutically effective combination of a compound of Formula I and an immune checkpoint inhibitor, wherein the cancer is not responsive to immune checkpoint inhibitor monotherapy.

In yet a further embodiment, a method for treating specific cancers comprising administering to a host in need thereof a MerTK inhibitor in combination or alternation with an inhibitor that prevents the downregulation of the immune system (immune checkpoint inhibitor), wherein the administration of the combination results in an additive inhibitory effect in the cancer compared to the use of either the MerTK inhibitor alone or the immune checkpoint inhibitor alone.

### Terminology

Compounds are described using standard nomenclature. Unless defined otherwise, all technical and scientific terms used herein have the same meaning as is commonly understood by one of skill in the art to which this invention belongs.

The compounds in any of the Formulas described herein include enantiomers, mixtures of enantiomers, diastereomers, tautomers, racemates and other isomers, such as rotamers, as if each is specifically described.

The terms “a” and “an” do not denote a limitation of quantity, but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed.

A dash (“-”) that is not between two letters or symbols is used to indicate a point of attachment for a substituent. For example, —(C═O)NH₂ is attached through carbon of the keto (C═O) group.

“Alkyl” as used herein alone or as part of another group, refers to a straight or branched chain hydrocarbon typically containing from 1 to 10 carbon atoms. In one embodiment, the alkyl contains from 1 to about 10 carbon atoms, more generally from 1 to about 6 carbon atoms or from 1 to about 4 carbon atoms. In certain embodiments, the alkyl is C₁-C₂, C₁-C₃, C₁-C₄, C₁-C₆, C₁-C₈, C₂-C₄, C₂-C₆, C₂-C₈, or C₄-C₆ The specified ranges as used herein indicate an alkyl group having each member of the range described as an independent species. For example, the term C₁-C₃ alkyl as used herein indicates a straight or branched alkyl group having from 1, 2, or 3 carbon atoms and is intended to mean that each of these is described as an independent species. For example, the term C₁-C₃alkyl as used herein indicates a straight or branched alkyl group having from 1, 2, or 3 carbon atoms and is intended to mean that each of these is described as an independent species. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. “Lower alkyl” as used herein, is a subset of alkyl, in some embodiments typically, and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, tert-butyl, and the like. In one embodiment, the alkyl or lower alkyl group is optionally substituted independently with one or more substituents described herein.

“Alkenyl” as used herein alone or as part of another group, refers to a straight or branched chain hydrocarbon typically containing from 1 to 10 carbon atoms (or in lower alkenyl 1 to 4 carbon atoms) which include 1 to 4 double bonds in the normal chain. Representative examples of alkenyl include, but are not limited to, vinyl, 2-propenyl, 3-butenyl, 2-butenyl, 4-pentenyl, 3-pentenyl, 2-hexenyl, 3-hexenyl, 2,4-heptadiene, and the like. In one embodiment, the alkenyl or lower alkenyl group is optionally substituted independently with one or more substituents described herein.

“Alkynyl” as used herein alone or as part of another group, refers to a straight or branched chain hydrocarbon typically containing from 1 to 10 carbon atoms (or in lower alkynyl 1 to 4 carbon atoms) which include 1 triple bond in the normal chain. Representative examples of alkynyl include, but are not limited to, 2-propynyl, 3-butynyl, 2-butynyl, 4-pentynyl, 3-pentynyl, and the like. In one embodiment, the alkynyl or lower alkynyl group is optionally substituted independently with one or more substituents described herein.

“Cycloalkyl” as used herein alone or as part of another group, refers to a saturated or partially unsaturated cyclic hydrocarbon group containing from 3, 4, 5, 6, 7 or 8 carbons. Representative examples of cycloalkyl include, cyclopropyl, cyclobutyl, cyclopentyl, cyclohexyl, cycloheptyl, and cyclooctyl. These rings may be optionally substituted with additional substituents as described herein such as halo or lower alkyl. In one embodiment, as used herein, the term “cycloalkyl” refers to a saturated or unsaturated hydrocarbon mono- or multi-ring, e.g., fused, bridged, or Spiro rings system having 3 to 15 carbon atoms (e.g., C₃-C₁₀). Examples of cycloalkyl include, but are not limited to, cyclopropyl, cyclobutyl, cyclopentyl, cyclohexyl, cycloheptyl, cyclooctyl, and adamantyl. In another embodiment, the term “cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆/C₅₋₆/C₃₋₈/C₄₋₈/C₅₋₈, and C₆₋₈. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2]bicyclooctane, decahydronaphthalene and adamantane. Cycloalkyl groups can also be partially unsaturated, having one or more double bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. In one embodiment, the cycloalkyl is a partially unsaturated (i.e., not aromatic) group containing all carbon ring atoms. In another embodiment, the cycloalkyl is a saturated group containing all carbon ring atoms. In one embodiment, the cycloalkyl group is optionally substituted independently with one or more substituents described herein.

“Heterocyclic group”, “heterocycle”, or “heterocyclo” as used herein refers to a saturated or a partially unsaturated (i.e., having one or more double and/or triple bonds within the ring without aromaticity) carbocyclic moiety of 3 to about 12, and more typically 3, 5, 6, 7 to 10 ring atoms in which at least one ring atom is a heteroatom selected from nitrogen, oxygen, phosphorus and sulfur, the remaining ring atoms being C, where one or more ring atoms is optionally substituted independently with one or more substituents described above. A heterocycle may be a monocycle having 3 to 7 ring members (2 to 6 carbon atoms and 1 to 4 heteroatoms selected from N, O, P, S, B or Si) or a bicycle having 6 to 10 ring members (4 to 9 carbon atoms and 1 to 6 heteroatoms selected from N, O, P, S, B or Si), for example: a bicyclo [4,5], [5,5], [5,6], or [6,6] system. In one embodiment, the only heteroatom is nitrogen. In one embodiment, the only heteroatom is oxygen. In one embodiment, the only heteroatom is sulfur. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. Examples of heterocyclic rings include, but are not limited to, pyrrolidinyl, dihydrofuranyl, tetrahydrothienyl, tetrahydropyranyl, dihydropyranyl, tetrahydrothiopyranyl, piperidino, piperidonyl, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, dihydroisoquinolinyl, tetrahydroisoquinolinyl, pyrazolidinylimidazolinyl, imidazolidinyl, 2-oxa-5-azabicyclo [2.2.2]octane, 3 -oxa-8-azabicyclo [3.2.1]octane, 8-oxa-3-azabicyclo [3.2.1]octane, 6-oxa-3-azabicyclo [3.1.1]heptane, 2-oxa-5-azabicyclo [2.2.1]heptane, 3-azabicyco [3.1.0]hexanyl, 3 -azabicyclo[4.1.0]heptanyl, azabicyclo [2.2 .2]hexanyl, 3H-indolyl, quinolizinyl, N-pyridyl ureas, and pyrrolopyrimidine. Spiro moieties are also included within the scope of this definition. Examples of a heterocyclic group wherein 1 or 2 ring carbon atoms are substituted with oxo (═O) moieties are pyrimidinonyl and 1,1-dioxo-thiomorpholinyl. In one embodiment, the heterocycle groups herein are optionally substituted independently with one or more substituents described herein.

“Heterocycloalkyl” is a saturated ring group. It may have, for example, 1, 2, 3, or 4 heteroatoms independently chosen from N, S, and O, with remaining ring atoms being carbon. In a typical embodiment, nitrogen is the heteroatom. Monocyclic heterocycloalkyl groups typically have from 3 to about 8 ring atoms or from 4 to 6 ring atoms. Nonlimiting examples of heterocycloalkyl groups include morpholinyl, piperazinyl, piperidinyl, and pyrrolinyl. In one embodiment, the heterocycloalkyl group is optionally substituted independently with one or more substituents described herein.

“Aryl” as used herein alone or as part of another group, refers to a monocyclic carbocyclic ring system or a bicyclic carbocyclic fused ring system having one or more aromatic rings. Representative examples of aryl include, azulenyl, indanyl, indenyl, naphthyl, phenyl, tetrahydronaphthyl, and the like. In one embodiment, the aryl group is optionally substituted independently with one or more substituents described herein.

“Arylalkyl” as used herein alone or as part of another group, refers to an aryl group, as defined herein, appended to the parent molecular moiety through an alkyl group, as defined herein. Representative examples of arylalkyl include, but are not limited to, benzyl, 2-phenylethyl, 3-phenylpropyl, 2-naphth-2-ylethyl, and the like.

“Heteroaryl” indicates a stable monocyclic aromatic ring which contains from 1 to 3, or in some embodiments from 1, 2 or 3 heteroatoms chosen from N, O, S, B or P with remaining ring atoms being carbon, or a stable bicyclic or tricyclic system containing at least one 4 to 7 or 5- to 7-membered aromatic ring which contains from 1 to 3, or in some embodiments from 1 to 2, heteroatoms chosen from N, O, S, B or P with remaining ring atoms being carbon. In one embodiment, the only heteroatom is nitrogen. In one embodiment, the only heteroatom is oxygen. In one embodiment, the only heteroatom is sulfur. Monocyclic heteroaryl groups typically have from 5 to 7 ring atoms. In some embodiments bicyclic heteroaryl groups are 9- to 10-membered heteroaryl groups, that is, groups containing 9 or 10 ring atoms in which one 5- to 7-member aromatic ring is fused to a second aromatic or non-aromatic ring. When the total number of S and O atoms in the heteroaryl group exceeds 1, these heteroatoms are not adjacent to one another. In one embodiment, the total number of S and O atoms in the heteroaryl group is not more than 2. In another embodiment, the total number of S and O atoms in the aromatic heterocycle is not more than 1. Examples of heteroaryl groups include, but are not limited to, pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, tetrahydrofuranyl, and furopyridinyl. Heteroaryl groups are optionally substituted independently with one or more substituents described herein.

“Alkoxy” as used herein alone or as part of another group, refers to an alkyl or lower alkyl group, as defined herein (and thus including substituted versions such as polyalkoxy), appended to the parent molecular moiety through an oxy group, —O—. Representative examples of alkoxy include, but are not limited to, methoxy, ethoxy, propoxy, 2-propoxy, butoxy, tert-butoxy, pentyloxy, hexyloxy and the like.

“Halo” as used herein refers to any suitable halogen, including —F, —Cl, —Br, and —I.

“Mercapto” as used herein refers to an —SH group.

“Cyano” as used herein refers to a —CN group.

“Carboxylic acid” as used herein refers to a —C(O)OH group.

“Hydroxyl” as used herein refers to an —OH group.

“Nitro” as used herein refers to an —NO₂ group.

“Acyl” as used herein alone or as part of another group refers to a —C(O)R radical, where R is any suitable substituent such as aryl, alkyl, alkenyl, alkynyl, cycloalkyl or other suitable substituent as described herein.

“Amino” as used herein means the radical —NH₂.

“Alkylamino” as used herein alone or as part of another group means the radical —NHR, where R is an alkyl group.

“Arylalkylamino” as used herein alone or as part of another group means the radical —NHR, where R is an arylalkyl group.

“Disubstituted-amino” as used herein alone or as part of another group means the radical —NRₐR_{b}, where Rₐ and R_{b} are independently selected from the groups hydrogen, alkyl, haloalkyl, alkenyl, alkynyl, cycloalkyl, cycloalkylalkyl, aryl, arylalkyl, heterocyclo, heterocycloalkyl.

“Acylamino” as used herein alone or as part of another group means the radical NRₐR_{b}, where Rₐ is an acyl group as defined herein and R_{b} is selected from the groups hydrogen, alkyl, haloalkyl, alkenyl, alkynyl, cycloalkyl, cycloalkylalkyl, aryl, arylalkyl, heterocyclo, heterocycloalkyl.

“Acyloxy” as used herein alone or as part of another group means the radical —OR, where R is an acyl group as defined herein.

“Ester” as used herein alone or as part of another group refers to a —C(O)OR radical, where R is any suitable substituent such as alkyl, cycloalkyl, alkenyl, alkynyl or aryl.

“Amide” as used herein alone or as part of another group refers to a —C(O)NRₐR_{b} radical, where Rₐ and R_{b} are any suitable substituent such as alkyl, cycloalkyl, alkenyl, alkynyl or aryl. In some embodiments, Rₐ and R_{b} together with the nitrogen to which they are bonded form a heterocyclic ring.

“Sulfonyl” as used herein refers to a compound of the formula —S(O)(O)R, where R is any suitable substituent such as amino, alkyl, cycloalkyl, alkenyl, alkynyl or aryl.

“Sulfonate” as used herein refers to a compound of the formula —S(O)(O)OR, where R is any suitable substituent such as alkyl, cycloalkyl, alkenyl, alkynyl or aryl.

“Sulfonic acid” as used herein refers to a compound of the formula —S(O)(O)OH.

“Sulfonamide” as used herein alone or as part of another group refers to a —S(O)₂NRₐR_{b} radical, where Rₐ and R_{b} are any suitable substituent such as H, alkyl, cycloalkyl, alkenyl, alkynyl or aryl. In some embodiments, Rₐ and R_{b} are any suitable substituent such as hydrogen, alkyl, alkenyl, alkynyl, aryl, arylalkyl, cycloalkyl, cycloalkylalkyl, heterocyclo, heterocycloalkyl, heteroaryl, or heteroarylalkyl and each Rₐ and R_{b} can be optionally substituted one, two or three times. In some embodiments, Rₐ and R_{b} together with the nitrogen to which they are bonded form a heterocyclic ring that can be optionally substituted one, two or three times.

“Urea” as used herein alone or as part of another group refers to an —N(R_{c})C(O)NRₐR_{b} radical, where Rₐ, R_{b} and R_{c} are any suitable substituent such as H, alkyl, cycloalkyl, alkenyl, alkynyl or aryl. In some embodiments, Rₐ and R_{b} together with the nitrogen to which they are bonded form a heterocyclic ring.

“Alkoxyacylamino” as used herein alone or as part of another group refers to an —N(Rₐ)C(O)OR_{b} radical, where Rₐ, R_{b} are any suitable substituent such as H, alkyl, cycloalkyl, alkenyl, alkynyl or aryl.

“Aminoacyloxy” as used herein alone or as part of another group refers to an —OC(O)NRₐR_{b} radical, where Rₐ and R_{b} are any suitable substituent such as H, alkyl, cycloalkyl, alkenyl, alkynyl or aryl. In some embodiments, Rₐ and R_{b} together with the nitrogen to which they are bonded form a heterocyclic ring.

“Optionally substituted” as used herein refers to the optionally substitution of a chemical moiety. These moieties can be substituted with groups selected from, but not limited to, halo (F, Cl, Br, and I), alkyl, haloalkyl, hydroxyalkyl, alkenyl, alkynyl, cycloalkyl (including spiroalkyl, e.g., —C(CH₂)₂₄— spiroalkyl), cycloalkylalkyl, aryl, arylalkyl, aryl substituted heteroaryl, heterocyclo, heterocycloalkyl, alkylheterocycloalkyl, heteroaryl, heteroarylalkyl, hydroxyl, alkoxy (thereby creating a polyalkoxy such as polyethylene glycol), alkenyloxy, alkynyloxy, haloalkoxy, cycloalkoxy, cycloalkylalkyloxy, aryloxy, arylalkyloxy, heterocyclooxy, heterocyclolalkyloxy, mercapto, alkyl-S(O)ₘ, haloalkyl-S(O)ₘ, alkenyl-S(O)ₘ, alkynyl-S(O)ₘ, cycloalkyl-S(O)ₘ, cycloalkylalkyl-S(O)ₘ, aryl-S(O)ₘ, arylalkyl-S(O)ₘ, heterocyclo-S(O)ₘ, heterocycloalkyl-S(O)ₘ, amino, carboxy, alkylamino, —(CH₂)ₘ—NH(CH₂)ₘCH₃, —(CH₂)ₘ—NH(CH₂)ₘOH, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro, or cyano where m=0, 1, 2 or 3. In one embodiment, these moieties can be substituted with groups selected from —(CH₂)ₘ—N(R⁵⁰)₂, —(CH₂)ₘ—NH(CH₂)ₘR⁵⁰, —(CH₂)ₘNH(CH₂)₂₋₃N(R⁵⁰)₂, —S(O)₂OR⁵⁰, —CONHNHR⁵⁰, aminosulfonyl —C(CH₂)₂R⁵⁰ wherein each R⁵⁰ is independently selected from hydrogen, alkyl, alkenyl, alkynyl, aryl, arylalkyl, cycloalkyl, cycloalkylalkyl, heterocyclo, heterocycloalkyl, heteroaryl, or heteroarylalkyl.

“Immune checkpoint” as used herein refers to a molecule on the cell surface of a CD4 and CD8 T cell that down-modulates or inhibits an anti-tumor immune response. Immune checkpoint molecules include, but are not limited to, Programmed Death 1 (PD1), Cytotoxic T-Lymphocyte Antigen 4 (CTLA-4), PDL-1 (B7H1), PDL-2 (B7-DC), B7H3, B7H4, OX-40, CD137, CD40, CD27, LAG3, TIM3, ICOS, or BTLA, which directly inhibit immune cells. Immunotherapeutic agents which can act as immune checkpoint inhibitors useful in the methods of the present invention include, but are not limited to, anti-PD1; anti-CTLA-4; anti-PDL-1; anti-B7-H1; anti-PDL-2; anti-B7-H3; anti-B7-H4; anti-CD137; anti-CD40; anti-CD27; anti-LAG3; anti-TIM3; anti-ICOS, and anti-BTLA.

The term “subtherapeutic dose” as used herein refers to a dose that is below the effective monotherapy dosage levels for the disorder being treated in the host being treated. In one nonlimiting embodiment, the subtherapeutic dose of a compound of Formula I, and in particular UNC2371, does not substantially affect the growth of the cancer or tumor being treated when administered alone. In an alternative nonlimiting embodiment, the subtherapeutic dose of an immune checkpoint inhibitor does not substantially affect the growth of the cancer or tumor being treated when administered alone.

A “dosage form” means a unit of administration of an active agent. Examples of dosage forms include but are not limited to tablets, capsules, injections, suspensions, liquids, emulsions, implants, particles, spheres, creams, ointments, suppositories, inhalable forms, transdermal forms, buccal, sublingual, topical, gel, mucosal, and the like.

A “pharmaceutical composition” is a composition comprising at least one active agent, such as a compound or salt of Formula I, and at least one other substance, such as another active agent or a carrier.

A “pharmaceutical combination” is a combination of at least two active agents which may be combined in a single dosage form or provided together in separate dosage forms with instructions that the active agents are to be used together to treat any disorder described herein.

A “pharmaceutically acceptable salt” is a derivative of the disclosed compounds in which the parent compound is modified by making inorganic and organic, suitably non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Typical acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in Remington's Pharmaceutical Sciences, 17th ed., Mack Publishing Company, Easton, Pa., p. 1418 (1985).

The term “carrier” in a pharmaceutical compositions/combinations of the invention refers to a diluent, excipient, or vehicle with which an active compound is provided.

A “pharmaceutically acceptable excipient” means an excipient that is useful in preparing a pharmaceutical composition/combination that is generally safe, suitably non-toxic and neither biologically nor otherwise inappropriate for administration to a host, and includes, in one embodiment, an excipient that is acceptable for veterinary use as well as human pharmaceutical use. A “pharmaceutically acceptable excipient” as used in the present application includes both one and more than one such excipient.

As used herein the term “ionizing radiation” refers to radiation of sufficient energy that, when absorbed by cells and tissues, can induce formation of reactive oxygen species and DNA damage. Ionizing radiation can include X-rays, gamma rays, and particle bombardment (e.g., neutron beam, electron beam, protons, mesons, and others). Radiation is generally measured in units of absorbed dose, such as the rad or gray (Gy), or in units of dose equivalence, such as rem or sievert (Sv).

The present invention is primarily focused on the treatment of a human subject or host, but the invention may be used to treat animals, such as mammalian subjects such as mice, rats, dogs, cats, livestock and horses for veterinary purposes, and for drug screening and drug development purposes. Subjects may be of any age, including infant, juvenile, adolescent, adult, and geriatric subjects.

### MerTK Inhibitors

MerTK inhibitors useful for administration in combination with immune checkpoint inhibitors as contemplated herein have the structure of Formula I:

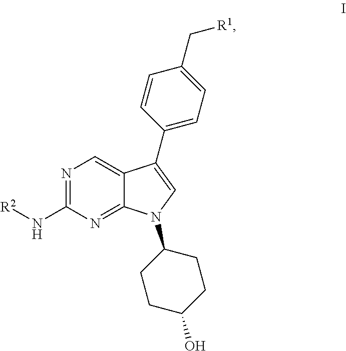

wherein R¹ is heterocyclo, wherein R¹ is optionally substituted one, two, or three times; and

R² is alkyl, cycloalkyl, or cycloalkylalkyl, wherein R² is optionally substituted one, two, or three times; or a pharmaceutically acceptable salt thereof.

In one embodiment, R¹ is a 5, 6, or 7 membered heterocycle containing 1 or 2 heteroatoms. In one embodiment, R¹ is a 6 membered heterocycle containing 1 or 2 heteroatoms. In one embodiment, R¹ is a 6 membered heterocycle containing 1 or 2 nitrogen atoms. In one embodiment, R¹ is a 6 membered heterocycle containing 2 nitrogen atoms. In one embodiment, R¹ is bound through a heteroatom, such as nitrogen.

In one embodiment, R¹ is a 5, 6, or 7 membered heterocycle containing 1 or 2 heteroatoms and substituted with C₁-C₄ alkyl. In one embodiment, R¹ is a 6 membered heterocycle containing 1 or 2 heteroatoms and substituted with C₁-C₄ alkyl. In one embodiment, R¹ is a 6 membered heterocycle containing 1 or 2 nitrogen atoms and substituted with C₁-C₄ alkyl. In one embodiment, R¹ is a 6 membered heterocycle containing 2 nitrogen atoms and substituted with C₁-C₄ alkyl. In one embodiment, R¹ is a 6 membered heterocycle containing 2 nitrogen atoms at the 1 and 4 positions of the heterocycle. In one embodiment, R¹ is a heterocycle bound through a nitrogen atom. In one embodiment, R¹ is a 6 membered heterocycle bound through a nitrogen atom.

In one embodiment, R¹ is a 5, 6, or 7 membered heterocycle containing 1 or 2 heteroatoms and substituted with methyl. In one embodiment, R¹ is a 6 membered heterocycle containing 1 or 2 heteroatoms and substituted with methyl. In one embodiment, R¹ is a 6 membered heterocycle containing 1 or 2 nitrogen atoms and substituted with methyl. In one embodiment, R¹ is a 6 membered heterocycle containing 2 nitrogen atoms and substituted with methyl. In one embodiment, R¹ is a 6 membered heterocycle containing 2 nitrogen atoms, wherein one of the nitrogen atoms is substituted with methyl.

In one embodiment, R¹ is C₃-C₈ heterocycle. In one embodiment, R¹ is a 5 membered heterocycle. In one embodiment, R¹ is a 5 membered heterocycle substituted with C₁-C₄ alkyl. In one embodiment, R¹ is a 5 membered heterocycle substituted with methyl. In one embodiment, R¹ is a 6 membered heterocycle. In one embodiment, R¹ is a 6 membered heterocycle substituted with C₁-C₄ alkyl. In one embodiment, R¹ is a 6 membered heterocycle substituted with methyl. In one embodiment, R¹ is a 6 membered heterocycle substituted with C₁-C₄ hydroxyalkyl. In one embodiment, R¹ is a 7 membered heterocycle. In one embodiment, R¹ is a 7 membered heterocycle substituted with C₁-C₄ alkyl. In one embodiment, R¹ is a 7 membered heterocycle substituted with methyl.

In one embodiment, R¹ is heterocycle, wherein the heterocycle is unsubstituted. In one embodiment, R¹ is heterocycle, wherein the heterocycle is substituted with alkyl. In one embodiment, R¹ is heterocycle, wherein the heterocycle is substituted with methyl. In one embodiment, R¹ is substituted with alkyl. In one embodiment, R¹ is substituted with methyl.

In one embodiment, R¹ is selected from the group consisting of piperazinyl, piperidinyl, and homopiperazinyl. In one embodiment, R¹ is piperazinyl. In one embodiment, R¹ is piperazinyl, substituted with alkyl. In one embodiment, R¹ is piperazinyl, substituted with methyl. In one embodiment, R¹ is N-methylpiperazinyl. In one embodiment, R¹ is 4-methylhomopiperazinyl. In one embodiment, R¹ is piperazinyl, wherein the piperazinyl is bound through a nitrogen atom. In one embodiment, R¹ is piperidinyl. In one embodiment, R¹ is piperidinyl, substituted with hydroxyalkyl. In one embodiment, R¹ is 2-hydroxyethylpiperidinyl. In one embodiment, R¹ is morpholinyl.

In one embodiment, R² is alkyl. In one embodiment, R² is lower alkyl. In one embodiment, R² is alkyl, wherein the alkyl is unsubstituted. In one embodiment, R² is C₁-C₈ alkyl. In one embodiment, R² is C₁-C₆ alkyl. In one embodiment, R² is C₁-C₄ alkyl. In one embodiment, R² is C₃-C₆ alkyl. In one embodiment, R² is selected from n-propyl, iso-propyl, n-butyl, sec-butyl, iso-butyl, tert-butyl, n-pentyl, isopentyl, neopentyl, and n-hexyl. In one embodiment, R² is n-butyl.

In one embodiment, R² is C₂-C₈ hydroxyalkyl. In one embodiment, R² is C₂-C₆ hydroxyalkyl. In one embodiment, R² is C₂-C₄ hydroxyalkyl. In one embodiment, R² is C₄ hydroxyalkyl. In one embodiment, R² is 4-hydroxylbutyl.

In one embodiment, R² is cycloalkylalkyl. In one embodiment, R² is cycloalkylalkyl, wherein the cycloalkylalkyl is unsubstituted. In one embodiment, R² is C₃-C₈ cycloalkylalkyl. In one embodiment, R² is C₂-C₄alkyl(C₃-C₈ cycloalkyl). In one embodiment, R² is C₂-C₄alkyl(C₃-C₆ cycloalkyl). In one embodiment, R² is C₂-C₄alkyl(C₃ cycloalkyl). In one embodiment, R² is C₂-C₃alkyl(C₃-C₅ cycloalkyl). In one embodiment, R² is C₂ alkyl(C₃-C₈ cycloalkyl). In one embodiment, R² is C₂ alkyl(C₃-C₆ cycloalkyl). In one embodiment, R² is C₂ alkyl(C₃ cycloalkyl). In one embodiment, R² is 2-cyclopropylethyl. In one embodiment, R² is 2-cyclobutylethyl. In one embodiment, R² is 2-cyclopentylethyl. In one embodiment, R² is 3-cyclopropylpropyl. In one embodiment, R² is 3-cyclobutylpropyl. In one embodiment, R² is 3-cyclopentylpropyl. In one embodiment, R² is C₃-C₈ cycloalkyl. In one embodiment, R² is C₅-C₆ cycloalkyl.

In one embodiment, the MerTK inhibitor administered to a host as contemplated herein is selected from the compounds of Table 1:

TABLE 1
MerTK Inhibitors

| | Structure | Compound_ID |
|---|---|---|
| 1 | 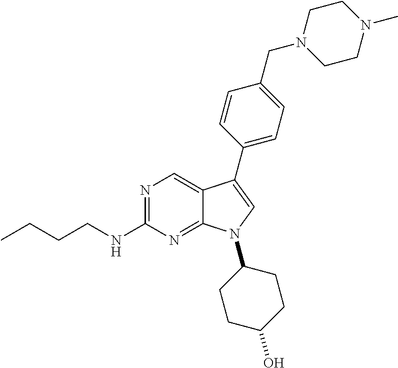 | UNC2025 |
| 2 | 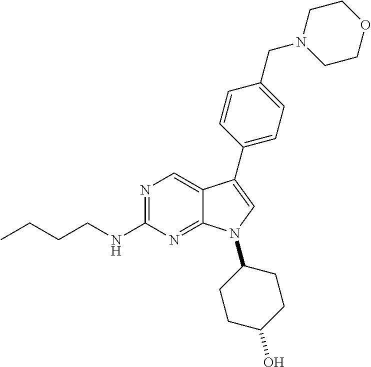 | UNC2142 |
| 3 | 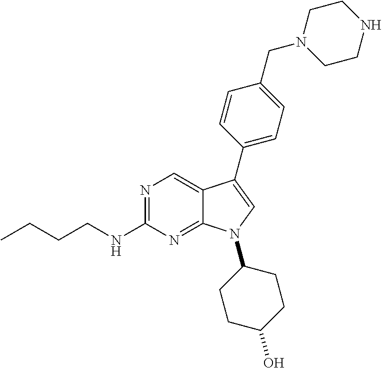 | UNC2143 |
| 4 | 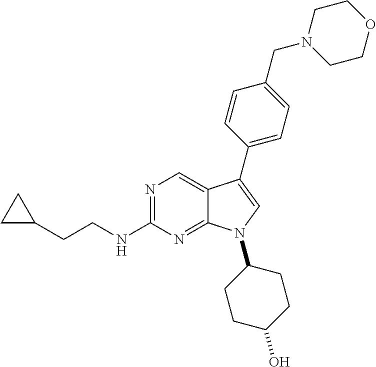 | UNC2370 |
| 5 | 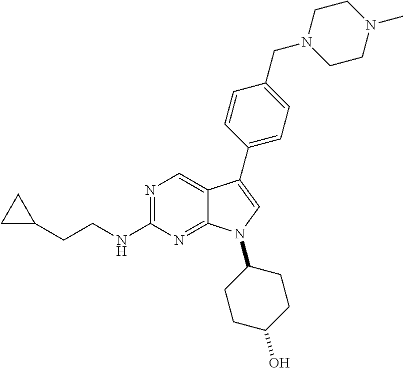 | UNC2371 |
| 6 | 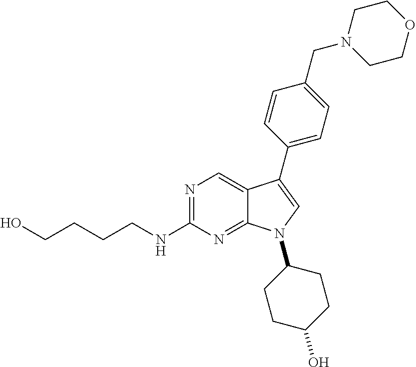 | UNC2395 |
| 7 | 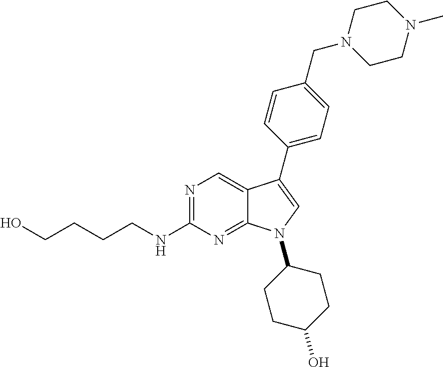 | UNC2396 |
| 8 | 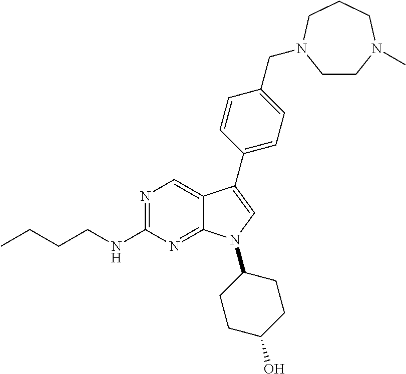 | UNC4218 |
| 9 | 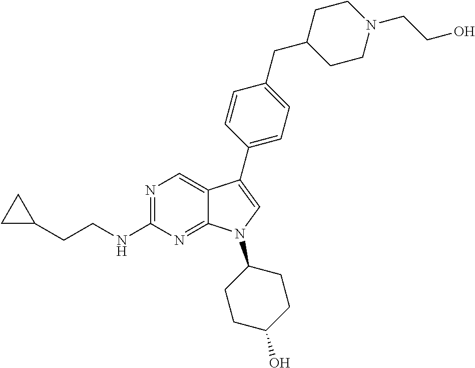 [SMILES: OCCN1CCC(Cc2ccc(-c3cn([C@H]4CC[C@H](O)CC4)c4nc(NCCC5CC5)ncc34)cc2)CC1] | UNC3908 |

Compounds UNC2025, UNC2142, UNC2143, UNC2370, UNC2371, UNC2395, and
UNC2396 were originally disclosed in WO 2013/052417. Compounds UNC4218 and
UNC3908 are disclosed in PCT/US2015/024381.

In one embodiment, the MerTK inhibitor has the structure:

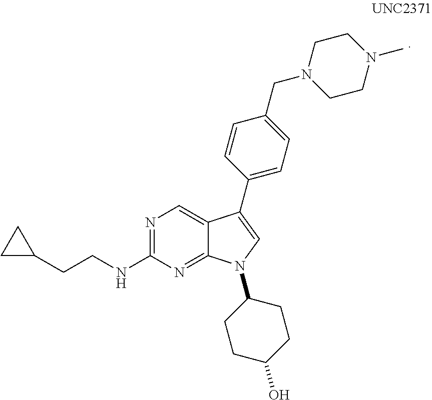

Active compounds may be provided as pharmaceutically acceptable prodrugs, which are, within the scope of sound medical judgment, suitable for use in contact with the tissues of humans and lower animals without undue toxicity, irritation, allergic response and the like, commensurate with a reasonable risk/benefit ratio, and effective for their intended use, as well as the zwitterionic forms, where possible, of the compounds of the invention.

The term “prodrug” refers to compounds that are transformed, sometimes rapidly in vivo to yield the parent compound of the above formulae, for example, by hydrolysis intracellularly or extracellularly, for example, in blood. A thorough discussion is provided in T. Higuchi and V. Stella, Prodrugs as Novel delivery Systems, Vol. 14 of the A.C.S.

Symposium Series and in Edward B. Roche, ed., Bioreversible Carriers in Drug Design, American Pharmaceutical Association and Pergamon Press, 1987, both of which are incorporated by reference herein. See also U.S. Pat. No. 6,680,299. Examples include a prodrug that is metabolized in vivo by a subject to an active drug having an activity of a compound as described herein, wherein the prodrug is an ester of an alcohol or carboxylic acid group, if such a group is present in the compound; an acetal or ketal of an alcohol group, if such a group is present in the compound; an N-Mannich base or an imine of an amine group, if such a group is present in the compound; or a Schiff base, oxime, acetal, enol ester, oxazolidine, or thiazolidine of a carbonyl group, if such a group is present in the compound, such as described in U.S. Pat. No. 6,680,324 and U.S. Pat. No. 6,680,322.

The MerTK inhibitors for use in the present invention as contemplated herein can, as noted above, be provided in the form of a pharmaceutically acceptable salt. Pharmaceutically acceptable salts are salts that retain the desired biological activity of the parent compound and do not impart an undesired toxicological effect. Nonlimiting examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

MerTK inhibitors, as described herein, can be prepared in accordance with known procedures, or variations thereof that will be apparent to those skilled in the art and as described in U.S. Patent Application Publication No. 2014/0243315.

### Immune Checkpoint Inhibitors

An immune checkpoint molecule is one that is capable of inhibiting or downmodulating an immune response to a tumor or cancer. An inhibitor of an immune checkpoint molecule is a small molecule (pharmaceutical) or large molecule (biologic) capable of turning off the down regulation of the immune system to the tumor or cancer. Examples of immune checkpoint inhibitors useful for administration in combination with MerTK inhibitors as contemplated herein include inhibitors of CTLA4, PD1, PDL-1, B7H1, B7H3, B7H4, OX-40, CD137, CD40, CD27, LAG3, TIM3, ICOS, or BTLA, including antibodies to these proteins.

In one embodiment, the immune checkpoint inhibitor is a CTLA4 inhibitor. Cytotoxic T-lymphocyte antigen 4 (CTLA4, also known as CD152) is a member of the immunoglobulin superfamily that is expressed exclusively on T-cells. CTLA4 acts to inhibit T-cell activation and is reported to inhibit helper T-cell activity and enhance regulatory T-cell immunosuppressive activity. Some anti-CTLA4 antibodies have been approved for the treatment of melanoma, prostate cancer, small cell lung cancer, non-small cell lung cancer (Pardoll, D. “The blockade of immune checkpoints in cancer immunotherapy.” 2012, Nature Reviews Cancer 12:252-264), and others have shown efficacy in clinical trials.

Non-limiting examples of anti-CTLA4 antibodies which can be used herein include ipilimumab (Yervoy®, MDX-010, Bristol-Myers Squibb) and tremelimumab (CP-675206, Pfizer).

In one embodiment, the immune checkpoint inhibitor is a PD-1 inhibitor. Programmed cell death protein 1 (PD1, also known as CD279) is a cell surface membrane protein of the immunoglobulin superfamily. The major role of PD1 is to limit the activity of T cells in peripheral tissues during inflammation in response to infection, as well as to limit autoimmunity. PD1 expression is induced in activated T cells and binding of PD1 to one of its endogenous ligands acts to inhibit T-cell activation by inhibiting stimulatory kinases. PD1 is highly expressed on T_{reg} cells and may increase their proliferation in the presence of ligand (Pardoll, D. “The blockade of immune checkpoints in cancer immunotherapy.” 2012, Nature Reviews Cancer 12:252-264).

Non-limiting examples of anti-PD1 antibodies contemplated for use herein include, but are not limited to, pembrolizumab (Keytruda®, MK-3475, formerly lambrolizumab, Merck), nivolumab (Opdivo®, BMS-936558, Bristol-Myers Squibb), AMP-224 (Merck), pidilizumab (CT-011, Curetech), and MEDI0680/AMP-514 (Astrazeneca/MedImmune).

Other immune checkpoint inhibitors that may be used including, for example, immune checkpoint inhibitors targeting PDL-1 (B7H1), PDL-2 (B7-DC), B7H3, B7H4, OX-40, CD137, CD40, CD27, LAG3, TIM3, ICOS, or BTLA (Pardoll, 2012, Nature Reviews Cancer 12:252-264).

In one embodiment, the immune checkpoint inhibitor is selected from the group consisting of a PDL-1 inhibitor, B7H1 inhibitor, B7H3 inhibitor, B7H4 inhibitor, OX-40 inhibitor, CD137 inhibitor, CD40 inhibitor, CD27 inhibitor, LAG3 inhibitor, TIM3 inhibitor, ICOS inhibitor, or BTLA inhibitor.

In one embodiment, the immune checkpoint inhibitor is a PDL-1 inhibitor. PDL-1 is expressed on cancer cells and causes the immune response shut down. Non-limiting examples of PDL-1 inhibitors that are useful in the present invention include MPDL3280A (Roche/Genentech), MEDI4736 (AstraZeneca/MedImmune), BMS-936559 (Bristol-Myers Squibb), and avelumab (MSB0010718; Merck/Pfizer). MPDL3280A (Roche/Genentech) has entered clinical trials for bladder cancer, non-small cell lung cancer, melanoma, kidney cancer, lymphoma, and solid tumors. MEDI4736 (AstraZenecalMedlmmune) is in clinical trials for a number of cancers, including brain, cervical, colorectal, head and neck, kidney, lung, and ovarian cancers.

In one embodiment, the immune checkpoint inhibitor is a 4-1BB inhibitor. 4-1BB, also known as CD137, is a costimulator for activated T cells. Non-limiting examples of 4-IBB inhibitors contemplated herein include urelumab (BMS-663513, Bristol-Myers Squibb) and PF-05082566 (PF-2566, Pfizer).

In one embodiment, the immune checkpoint inhibitor is a LAG-3 inhibitor. Non-limiting examples of LAG-3 inhibitors contemplated for use herein include BMS-986016 and IMP321. A LAG-3 antibody (BMS-986016, Bristol-Myers Squibb) is being tested in patients with hematological and solid cancers. IMP321 (Prima BioMed) is a soluble version of the LAG3 molecule.

In one embodiment, the immune checkpoint inhibitor is a CD27 inhibitor. The CD27 costimulatory molecule plays an important role in the activation, survival, and differentiation of T cells. One non-limiting example of an CD27 inhibitor contemplated for use herein is varlilumab (CDX-1127, Celldex) and is being tested in B cell cancers, T cell cancers, and solid tumors, including melanoma, kidney cancer, prostate cancer, ovarian cancer, colorectal cancer, and lung cancer.

In one embodiment, the immune checkpoint inhibitor is a CD40 inhibitor. CD40 is an activating protein on the surface of B cells and activates dendritic cells to promote CD8+ T cell activation and proliferation. One non-limiting example of a CD40 inhibitor contemplated for use herein includes CP-870,893 (Pfizer) and is being tested for pancreatic cancer.

In one embodiment, the immune checkpoint inhibitor is a B7-H3 inhibitor. One non-limiting example of a B7-H3 inhibitor contemplated for use herein is MGA271. MGA271 (Macrogenics) entered clinical trials for multiple cancers.

### Toll-Like Receptor (TLR) Agonists

Toll-like receptors (TLRs) play a cital role in activating immune responses. TLRs recognize conserved pathogen-associated molecular patterns (PAMPs) expressed in a wide array of microbes, as well as endogenous damage-associated molecular patterns (DAMPs) released from stressed or dying cells. There has been a major effort in recent years, with significant success, to discover new drug compounds that act by stimulating certain key aspects of the immune system, as well as by suppressing certain other aspects (see, e.g., U.S. Pat. Nos. 6,039,969 and 6,200,592). These compounds appear to act through basic immune system mechanisms known as Toll-like receptors (TLRs) and are referred to herein as “TLR agonists.”

Many TLR agonists are small organic molecule imidazoquinoline amine derivatives (see, e.g., U.S. Pat. No. 4,689,338), but a number of other compound classes are known as well (see, e.g., U.S. Pat. Nos. 5,446,153; 6,194,425; and 6,110,929; and International Publication Number WO 2005/079195) and more are still being discovered. Other IRMs have higher molecular weights, such as oligonucleotides, including CpGs (see, e.g., U.S. Pat. No. 6,194,388).

TLR agonists are known in the art and include small organic molecules (e.g., molecular weight under about 1000 Daltons, preferably under about 500 Daltons, as opposed to large biological molecules such as proteins, peptides, and the like) such as those disclosed in, for example, U.S. Pat. Nos. 4,689,338; 4,929,624; 5,266,575; 5,268,376; 5,346,905; 5,352,784; 5,389,640; 5,446,153; 5,482,936; 5,756,747; 6,110,929; 6,194,425; 6,331,539; 6,376,669; 6,451,810; 6,525,064; 6,541,485; 6,545,016; 6,545,017; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; 6,797,718; 6,818,650; and 7,7091,214; U.S. Patent Publication Nos. 2004/0091491, 2004/0176367, and 2006/0100229; and International Publication Nos. WO 2005/18551, WO 2005/18556, WO 2005/20999, WO 2005/032484, WO 2005/048933, WO 2005/048945, WO 2005/051317, WO 2005/051324, WO 2005/066169, WO 2005/066170, WO 2005/066172, WO 2005/076783, WO 2005/079195, WO 2005/094531, WO 2005/123079, WO 2005/123080, WO 2006/009826, WO 2006/009832, WO 2006/026760, WO 2006/028451, WO 2006/028545, WO 2006/028962, WO 2006/029115, WO 2006/038923, WO 2006/065280, WO 2006/074003, WO 2006/083440, WO 2006/086449, WO 2006/091394, WO 2006/086633, WO 2006/086634, WO 2006/091567, WO 2006/091568, WO 2006/091647, WO 2006/093514, and WO 2006/098852. Additional examples of small molecule IRMs include certain purine derivatives (such as those described in U.S. Pat. Nos. 6,376,501, and 6,028,076), certain imidazoquinoline amide derivatives (such as those described in U.S. Pat. No. 6,069,149), certain imidazopyridine derivatives (such as those described in U.S. Pat. No. 6,518,265), certain benzimidazole derivatives (such as those described in U.S. Pat. No. 6,387,938), certain derivatives of a 4-aminopyrimidine fused to a five membered nitrogen containing heterocyclic ring (such as adenine derivatives described in U.S. Pat. Nos. 6,376,501; 6,028,076 and 6,329,381; and in WO 02/08905), and certain 3-β-D-ribofuranosylthiazolo[4,5-d]pyrimidine derivatives (such as those described in U.S. Publication No. 2003/0199461), and certain small molecule immuno-potentiator compounds such as those described, for example, in U.S. Patent Publication No. 2005/0136065.

Other TLR agonists include large biological molecules such as oligonucleotide sequences. Some TLR agonist oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other TLR agonist nucleotide sequences lack CpG sequences and are described, for example, in International Patent Publication No. WO 00/75304. Still other TLR agonist nucleotide sequences include guanosine- and uridine-rich single-stranded RNA (ssRNA) such as those described, for example, in Heil et ah, Science, vol. 303, pp. 1526-1529, Mar. 5, 2004.

Other TLR agonists include biological molecules such as aminoalkyl glucosaminide phosphates (AGPs) and are described, for example, in U.S. Pat. Nos. 6,113,918; 6,303,347; 6,525,028; and 6,649,172.

Further TLR agonists useful in the present invention may include those described herein below.

TLR2/1 agonists and TLR2/6 agonists: TLR2 is typically a heteromeric receptor found in combination with either TLR1 or TLR6. Bacterial lipopeptides are the main agonists for TLR2-containing receptors. These agonists include: mycoplasmal macrophage-activating lipoprotein-2; tripalmitoyl-cysteinyl-seryllysyl)3-lysine (P3CSK4), dipalmitoyl-CSK4 (P2-CSK4), and monopalmitoyl-CSK4 (PCSK4); the tripalmitoyl-S-glyceryl-cysteine (Pam(3)Cys)-modified lipoproteins, including OspA from the Lyme disease spirochete Borrelia burgdorferi; mycobacterial cell wall fractions enriched for lipoarrabinomannan, mycolylarabinogalactan-peptidoglycan complex, or M. tuberculosis total lipids.

TLR3 agonists: TLR3 agonists signal through the TRIF pathway to generate cytokines. The administration of viral genomes or partial genomes that generate dsRNA is another means of activating these pathways. In some cases, even endogenous messenger RNA (mRNA) can stimulate TLR3, and bacterial RNA can be especially stimulatory for dendritic cells. It has also been suggested that RNA stimulates dendritic cells through a nucleotide receptor. While viral double stranded RNAs (dsRNAs) can be used to stimulate TLR3, the best tested TLR3 agonist is polyriboinosinic-polyribocytidylic acid or Poly(I:C) which is a synthetic form of dsRNA. Poly(I:C) has antitumor effects in mice at a dose of 100 ug intraperitoneally or intravenously and has been extensively tested in humans with cancer. Poly(I:C) was shown to ameliorate herpes simplex keratoconjunctivitis in mice and to reduce the growth of Leishmania in mouse cells. For peptide vaccination, Poly(I:C) was used at a dose of 50 ug subcutaneously. In humans with herpes simplex infection and cancer, Poly(I:C) has been used at a dose of 3-12 mg/kg. Ampligen (poly I:poly C12U) is a mismatched form of dsRNA that has also been tested.

TLR4 agonists: TLR4 can signal cells through both the MyD88 and the TRIF pathways. Its special utility in activating human dendritic cells is art recognized. The classic agonist for TLR4 is bacterial lipopolysaccharide (LPS), which refers to a family of substances containing lipid A and its cogeners. An exemplary form of LPS is E. coli B:0111 (Sigma Chemicals). However, in an effort to make a less toxic form of TLR4 agonist, monophosphoryl lipid A (MPL) compounds have been produced and some are active in humans. The synthetic adjuvant, ASO2 (GlaxoSmithKline, United Kingdom), contains MPL as a component.

TLR5 agonists: The principal agonist for TLR5 is bacterial flagellin.

TLR7 agonists: For TLR7 agonists, these include, but are not limited to, single-stranded RNA; imidazoquinoline compounds such as resiquimod and imiquimod; Loxoribine (7-allyl-7,8-dihydro-8-oxo-guanosine) and related compounds; 7-Thia-8-oxoguanosine, 7-deazaguanosine, and related guanosine analogs; ANA975 (Anadys Pharmaceuticals) and related compounds; SM-360320 (Sumimoto); 3M-01 and 3M-03 (3M Pharmaceuticals); and adenosine analogs such as UC-1V150 (Jin et al., Bioorganic Medicinal Chem Lett (2006) 16:4559-4563, compound 4). It has been observed that TLR7 agonists directly activate plasmacytoid dendritic cells to make IFN-alpha, whereas TLR8 agonists directly activate myeloid dendritic cells, monocytes, and monocyte-derived dendritic cells to make proinflammatory cytokines and chemokines, such as TNF, IL-12, and MIP-1. Nevertheless, many compounds are agonists for both TLR7 and TLR8.

TLR8 agonists: As noted above, many of the compounds that activate TLR7 also activate TLR8. 3M-03 activates both TLR7 and TLR8, but 3M-02 is more specific for TLR8. Again, many compounds are agonists for both TLR7 and TLR8. Poly-G containing 10 guanosine nucleosides connected by phosphorothioate linkages (Poly-G10) is also a TLR8 agonist that may be especially useful as a substance that shuts off the immunosuppressive functions of regulator CD4+CD25+T cells.

TLR9 agonists: Immunostimulatory oligonucleotides or polynucleotides such as CpG-containing oligodeoxynucleotides (CpG ODN) are the prototype agonists for TLR9. More generally, they are called immunostimulatory sequences of oligodeoxynucleotides (ISS-ODN) because many immunostimulatory oligonucleotides (ODNs) do not contain a CpG motif. Typically, the ODN is a synthetic thiophosphorylate-linked compound. However, many types of DNA and RNA can activate TLR9 including bacterial DNA, liposomal vertebrate DNA, insect DNA, chlamydia polynucleotides and others.

Another class of TLR9 agonists are nucleotide sequences containing a synthetic cytosine-phosphate-2′-deoxy-7-deazaguanosine dinucleotide (CpR), called immunomodulatory oligonucleotides (IMOs) (Hybridon, Inc.). A dumbbell-like covalently-closed structure is also art recognized (dSLIM-30L1) that is an agonist for TLR9. PolyG oligodeoxynucleotides can also be immunostimulatory. Even double-stranded DNA, such as that released from dying cells, can increase an immune response. Plasmid DNA may be especially immunostimulatory. While this may be due to CpG motifs, it is not clear if this is always due to its agonistic activity for TLR9.

TLR11 agonists: One agonist for TLR11 is the profilin-like molecule from the protozoan parasite Toxoplasma gondii (PFTG).

In embodiments contemplated herein, the TLR agonist used as described above can be selected from triacylated lipoproteins, lipoteichoic acid, peptidoglycans, zymosan, Pam3CSK4, diacylated lipopeptides, heat shock proteins, HMBG1, uric acid, fibronectin, ECM proteins, MALP2, RC-529, dsRNA, Poly I:C, Mycobacterium bovis, (Bacillus-Calmette Guérin, BCG), Poly A:U, LPS, MDFbeta-2, beta-defensin 2, fibronectin EDA, snapin, tenascin C, MPL, flagellin, ssRNA, CpG-A, Poly G10, Poly G3, imiquimod 852A (Aldera), unmethylated CpG DNA, PamCysPamSK4, Toxoplasma gondii profiling, Loxoribine, or VSV. In specific embodiments, the TLR agonist is monophosphoryl lipid A (MPL), Mycobacterium bovis (Bacillus-Calmette Guérin, BCG), CpG, ISCOMatrix, imiquimod (Aldera), Poly IC:LC, OK-432, and/or resiquimod.

### Ionizing Radiation

Ionizing radiation (IR) is an important therapeutic modality to treat a range of cancers and other proliferative disorders such as tumors. Radiation therapy uses high energy radiation to shrink tumors and kill the proliferating cells. X-rays, gamma rays, and charged particles are typical kinds of ionizing radiation used for cancer treatments. IR causes extensive DNA damage to abnormally proliferating cells such as cancer and tumor cells.

As contemplated herein, a MerTk inhibitor and immune checkpoint inhibitor can be further combined or alternated with ionizing radiation treatments directed at a particular cancer. For example, the combinations contemplated herein can be combined or alternated with standard of care radiation treatments based on the particular cancer a host may be suffering from. Certain standard of care radiation modalities may include those wherein the subject is exposed to IR at least 5 times a week, at least 4 times a week, at least 3 times a week, at least 2 times a week, at least 1 time a week, at least 3 times a month, at least 2 times a month, or at least 1 time a month.

### Tumors and Methods of Their Treatment

The methods contemplated herein are useful for treating a host suffering from a tumor or cancer wherein the administration of the combination results in an additive proliferation inhibitory, growth inhibitory, or growth delayed effect in the cancer compared to the use of either the MerTK inhibitor alone or the immune checkpoint inhibitor alone. In some embodiments, the cancer treated may be a cancer that otherwise is not responsive to immune downregulation inhibitor monotherapy, that is, a cancer wherein its growth or proliferation is not significantly, substantially, or markedly inhibited or delayed by the administration of an immune checkpoint inhibitor alone.

As contemplated herein, the cancer treated can be a primary tumor or a metastatic tumor. For example, the methods described herein can be used to treat a solid tumor, for example, melanoma, lung cancer (including lung adenocarcinoma, basal cell carcinoma, squamous cell carcinoma, large cell carcinoma, bronchioloalveolar carcinoma, bronchiogenic carcinoma, non-small-cell carcinoma, small cell carcinoma, mesothelioma); breast cancer (including ductal carcinoma, lobular carcinoma, inflammatory breast cancer, clear cell carcinoma, mucinous carcinoma, serosal cavities breast carcinoma); colorectal cancer (colon cancer, rectal cancer, colorectal adenocarcinoma); anal cancer; pancreatic cancer (including pancreatic adenocarcinoma, islet cell carcinoma, neuroendocrine tumors); prostate cancer; prostate adenocarcinoma; ovarian carcinoma (ovarian epithelial carcinoma or surface epithelial-stromal tumor including serous tumor, endometrioid tumor and mucinous cystadenocarcinoma, sex-cord-stromal tumor); liver and bile duct carcinoma (including hepatocellular carcinoma, cholangiocarcinoma, hemangioma); esophageal carcinoma (including esophageal adenocarcinoma and squamous cell carcinoma); oral and oropharyngeal squamous cell carcinoma; salivary gland adenoid cystic carcinoma; bladder cancer; bladder carcinoma; carcinoma of the uterus (including endometrial adenocarcinoma, ocular, uterine papillary serous carcinoma, uterine clear-cell carcinoma, uterine sarcomas and leiomyosarcomas, mixed mullerian tumors); glioma, glioblastoma, medulloblastoma, and other tumors of the brain; kidney cancers (including renal cell carcinoma, clear cell carcinoma, Wilm's tumor); cancer of the head and neck (including squamous cell carcinomas); cancer of the stomach (gastric cancers, stomach adenocarcinoma, gastrointestinal stromal tumor); testicular cancer; germ cell tumor; neuroendocrine tumor; cervical cancer; carcinoids of the gastrointestinal tract, breast, and other organs; signet ring cell carcinoma; mesenchymal tumors including sarcomas, fibrosarcomas, haemangioma, angiomatosis, haemangiopericytoma, pseudoangiomatous stromal hyperplasia, myofibroblastoma, fibromatosis, inflammatory myofibroblastic tumor, lipoma, angiolipoma, granular cell tumor, neurofibroma, schwannoma, angiosarcoma, liposarcoma, rhabdomyosarcoma, osteosarcoma, leiomyoma, leiomysarcoma, skin, including melanoma, cervical, retinoblastoma, head and neck cancer, pancreatic, brain, thyroid, testicular, renal, bladder, soft tissue, adenal gland, urethra, cancers of the penis, myxosarcoma, chondrosarcoma, osteosarcoma, chordoma, malignant fibrous histiocytoma, lymphangiosarcoma, mesothelioma, squamous cell carcinoma; epidermoid carcinoma, malignant skin adnexal tumors, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma anaplastic; glioblastoma multiforme, neuroblastoma, medulloblastoma, malignant meningioma, malignant schwannoma, neurofibrosarcoma, parathyroid carcinoma, medullary carcinoma of thyroid, bronchial carcinoid, pheochromocytoma, Islet cell carcinoma, malignant carcinoid, malignant paraganglioma, melanoma, Merkel cell neoplasm, cystosarcoma phylloide, salivary cancers, thymic carcinomas, and cancers of the vagina among others.

The methods described herein can also be used for treating a host suffering from a lymphoma or lymphocytic or myelocytic proliferation disorder or abnormality. For example, the cancer can be a Hodgkin Lymphoma of a Non-Hodgkin Lymphoma. For example, the subject can be suffering from a Non-Hodgkin Lymphoma such as, but not limited to: an AIDS-Related Lymphoma; Anaplastic Large-Cell Lymphoma; Angioimmunoblastic Lymphoma; Blastic NK-Cell Lymphoma; Burkitt's Lymphoma; Burkitt-like Lymphoma (Small Non-Cleaved Cell Lymphoma); Chronic Lymphocytic Leukemia/Small Lymphocytic Lymphoma; Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma; Mantle Cell Lymphoma; Marginal Zone Lymphoma; Nasal T-Cell Lymphoma; Pediatric Lymphoma; Peripheral T-Cell Lymphomas; Primary Central Nervous System Lymphoma; T-Cell Leukemias; Transformed Lymphomas; Treatment-Related T-Cell Lymphomas; or Waldenstrom's Macroglobulinemia.

Alternatively, the subject may be suffering from a Hodgkin Lymphoma, such as, but not limited to: Nodular Sclerosis Classical Hodgkin's Lymphoma (CHL); Mixed Cellularity CHL; Lymphocyte-depletion CHL; Lymphocyte-rich CHL; Lymphocyte Predominant Hodgkin Lymphoma; or Nodular Lymphocyte Predominant HL.

In one embodiment, the methods as described herein may be useful to treat a host suffering from a specific T-cell, a B-cell, or a NK-cell based lymphoma, proliferative disorder, or abnormality. For example, the subject can be suffering from a specific T-cell or NK-cell lymphoma, for example, but not limited to: Peripheral T-cell lymphoma, for example, peripheral T-cell lymphoma and peripheral T-cell lymphoma not otherwise specified (PTCL-NOS); anaplastic large cell lymphoma, for example anaplastic lymphoma kinase (ALK) positive, ALK negative anaplastic large cell lymphoma, or primary cutaneous anaplastic large cell lymphoma; angioimmunoblastic lymphoma; cutaneous T-cell lymphoma, for example mycosis fungoides, Sézary syndrome, primary cutaneous anaplastic large cell lymphoma, primary cutaneous CD30+ T-cell lymphoproliferative disorder; primary cutaneous aggressive epidermotropic CD8+ cytotoxic T-cell lymphoma; primary cutaneous gamma-delta T-cell lymphoma; primary cutaneous small/medium CD4+ T-cell lymphoma, and lymphomatoid papulosis; Adult T-cell Leukemia/Lymphoma (ATLL); Blastic NK-cell Lymphoma; Enteropathy-type T-cell lymphoma; Hematosplenic gamma-delta T-cell Lymphoma; Lymphoblastic Lymphoma; Nasal NK/T-cell Lymphomas; Treatment-related T-cell lymphomas; for example lymphomas that appear after solid organ or bone marrow transplantation; T-cell prolymphocytic leukemia; T-cell large granular lymphocytic leukemia; Chronic lymphoproliferative disorder of NK-cells; Aggressive NK cell leukemia; Systemic EBV+ T-cell lymphoproliferative disease of childhood (associated with chronic active EBV infection); Hydroa vacciniforme-like lymphoma; Adult T-cell leukemia/lymphoma; Enteropathy-associated T-cell lymphoma; Hepatosplenic T-cell lymphoma; or Subcutaneous panniculitis-like T-cell lymphoma.

Alternatively, the subject may be suffering from a specific B-cell lymphoma or proliferative disorder such as, but not limited to: multiple myeloma; Diffuse large B cell lymphoma; Follicular lymphoma; Mucosa-Associated Lymphatic Tissue lymphoma (MALT); Small cell lymphocytic lymphoma; Mantle cell lymphoma (MCL); Burkitt lymphoma; Mediastinal large B cell lymphoma; Waldenstrom macroglobulinemia; Nodal marginal zone B cell lymphoma (NMZL); Splenic marginal zone lymphoma (SMZL); Intravascular large B-cell lymphoma; Primary effusion lymphoma; or Lymphomatoid granulomatosis; Chronic lymphocytic leukemia/small lymphocytic lymphoma; B-cell prolymphocytic leukemia; Hairy cell leukemia; Splenic lymphoma/leukemia, unclassifiable; Splenic diffuse red pulp small B-cell lymphoma; Hairy cell leukemia-variant; Lymphoplasmacytic lymphoma; Heavy chain diseases, for example, Alpha heavy chain disease, Gamma heavy chain disease, Mu heavy chain disease; Plasma cell myeloma; Solitary plasmacytoma of bone; Extraosseous plasmacytoma; Primary cutaneous follicle center lymphoma; T cell/histiocyte rich large B-cell lymphoma; DLBCL associated with chronic inflammation; Epstein-Barr virus (EBV)+ DLBCL of the elderly; Primary mediastinal (thymic) large B-cell lymphoma; Primary cutaneous DLBCL, leg type; ALK+ large B-cell lymphoma; Plasmablastic lymphoma; Large B-cell lymphoma arising in HHV8-associated multicentric; Castleman disease; B-cell lymphoma, unclassifiable, with features intermediate between diffuse large B-cell lymphoma and Burkitt lymphoma; B-cell lymphoma, unclassifiable, with features intermediate between diffuse large B-cell lymphoma and classical Hodgkin lymphoma; Nodular sclerosis classical Hodgkin lymphoma; Lymphocyte-rich classical Hodgkin lymphoma; Mixed cellularity classical Hodgkin lymphoma; or Lymphocyte-depleted classical Hodgkin lymphoma.

The methods described herein can be used to treat a subject suffering from a leukemia. For example, the subject may be suffering from an acute or chronic leukemia of a lymphocytic or myelogenous origin, such as, but not limited to: Acute lymphoblastic leukemia (ALL); Acute myelogenous leukemia (AML); Chronic lymphocytic leukemia (CLL); Chronic myelogenous leukemia (CML); juvenile myelomonocytic leukemia (JMML); hairy cell leukemia (HCL); acute promyelocytic leukemia (a subtype of AML); T-cell prolymphocytic leukemia (TPLL); large granular lymphocytic leukemia; or Adult T-cell chronic leukemia; large granular lymphocytic leukemia (LGL). In one embodiment, the patient suffers from an acute myelogenous leukemia, for example an undifferentiated AML (MO); myeloblastic leukemia (M1; with/without minimal cell maturation); myeloblastic leukemia (M2; with cell maturation); promyelocytic leukemia (M3 or M3 variant [M3V]); myelomonocytic leukemia (M4 or M4 variant with eosinophilia [M4E]); monocytic leukemia (M5); erythroleukemia (M6); or megakaryoblastic leukemia (M7).

In one embodiment, the methods described herein can be used to treat a host suffering from Acute Myeloid Leukemia (AML). In one embodiment, the AML contains a wild type FLT3 protein. In one embodiment, the replication of the AML cells are dependent on FLT3 expression for proliferation. In one embodiment, the AML contains a FLT3-ITD mutation. In one embodiment, the AML contains a FLT3-TKD mutation. In one embodiment, the AML contains both a FLT3-ITD and FLT3-TKD mutation.

FLT3-ITD mutations are well known in the art. FLT3-TKD mutations are also well known in the art. In one embodiment, a MerTK inhibitor in combination with an immune checkpoint inhibitor is administered to a host suffering from AML, wherein the AML contains a mutation within the FLT3-TKD at amino acid F691 or D835. In one embodiment, the FLT3-TKD mutation is selected from D835H, D835N, D835Y, D835A, D835V, D835V, D835E, I836F, I836L, I836V, I836D, I836H, I836M, and F691L. In one embodiment, the host is suffering from the FLT3-TKD mutation D835Y. In one embodiment, the host is suffering from the FLT3-TKD mutation F691L.

In one embodiment, the host is suffering from acute promyelocytic leukemia (a subtype of AML); a minimally differentiated AML (MO); myeloblastic leukemia (M1; with/without minimal cell maturation); myeloblastic leukemia (M2; with cell maturation); promyelocytic leukemia (M3 or M3 variant [M3V]); myelomonocytic leukemia (M4 or M4 variant with eosinophilia [M4E]); monocytic leukemia (M5); erythroleukemia (M6); or megakaryocytic leukemia (M7). In one embodiment, the host is suffering from AML that has relapsed or become refractory to previous treatments. In one embodiment, the host has previously been treated with a FLT3 inhibitor or other chemotherapeutic agent.

In one embodiment, the host is suffering from AML having both FLT3-ITD and FLT3-TKD mutations, wherein resistance to other FLT3 inhibitors, for example, AC220, has been established. In one embodiment, the host has an AML tumor comprising a FLT3 mutation, wherein the mutation has conferred resistance to quizartinib (AC220) or other FLT3 inhibitor selected from lestaurtinib, sunitinib, sorafenib, tandutinib, midostaurin, amuvatinib crenolanib, dovitinib, ENMD-2076 (Entremed), or KW-2449 (Kyowa Hakko Kirin), or a combination thereof.

In one embodiment, the cancer treated overexpresses MerTK. In one embodiment, the cancer, which overexpresses MerTK is selected from the group consisting of acute myeloid leukemia, T-cell acute lymphoid leukemia, B-cell acute lymphoid leukemia, lung cancer, glioma, melanoma, prostate cancer, schwannoma, mantle cell lymphoma, and rhabdomyosarcoma. In an alternative embodiment, the cancer ectopically expresses MerTK.

In one embodiment, the cancer treated has a mutation in the amino acid sequence of the MerTK extracellular or transmembrane domain selected from P4OS (melanoma), S159F (lung), E204K (urinary tract) S428G (gastric), I431F (lung), A446G (kidney), N454S (liver), W485S/C (lymphoma), and V486I (melanoma). In one embodiment the cancer treated has a mutation in the amino acid sequence of the MerTK cytosolic domain mutation selected from L586F (urinary tract), G594R (breast), S626C (urinary tract), P672S (lung), L688M (colon), A708S (head and neck), N718Y (lung), R722stop (colon), M790V (lung), P802S (melanoma), V873I (liver), S905F (lung), K923R (melanoma), P958L (kidney), D983N (liver), and D990N (colon).

In one embodiment, the cancer is a MerTK-negative (−/−) cancer. In one embodiment, a compound of Formula I, as described herein, in combination with an immune checkpoint inhibitor, is provided for use in treating colon cancer. In one embodiment, the immune checkpoint inhibitor is an anti-programmed cell death −1 (PD1) antibody. In one embodiment, the immune checkpoint inhibitor is an anti-CTLA4 antibody. In one embodiment, the MerTK inhibitor administered is selected from the compounds of Table 1. In one embodiment, the MerTK inhibitor administered is UNC2371. In one embodiment, UNC2371 is administered in combination with an anti-PD1 antibody. In one embodiment, UNC2371 is administered in combination with pembrolizumab. In one embodiment, UNC2371 is administered in combination with nivolumab. In one embodiment, UNC2371 is administered in combination with an anti-CTLA4 antibody. In one embodiment, UNC2371 is administered in combination with ipilimumab. In one embodiment, the MerTK inhibitor and the immune checkpoint inhibitor are further combined with a Toll-like receptor (TLR) agonist and/or ionizing radiation.

In one embodiment, a compound of Formula I, as described herein, in combination with an immune checkpoint inhibitor, is provided for use in treating a non-small cell lung carcinoma (NSCLC). In one embodiment, the immune checkpoint inhibitor is an anti-programmed cell death −1 (PD1) antibody. In one embodiment, the immune checkpoint inhibitor is an anti-CTLA4 antibody. In one embodiment, the MerTK inhibitor administered is selected from the compounds of Table 1. In one embodiment, the MerTK inhibitor administered is UNC2371. In one embodiment, UNC2371 is administered in combination with an anti-PD1 antibody. In one embodiment, UNC2371 is administered in combination with pembrolizumab. In one embodiment, UNC2371 is administered in combination with nivolumab. In one embodiment, UNC2371 is administered in combination with an anti-CTLA4 antibody. In one embodiment, UNC2371 is administered in combination with ipilimumab. In one embodiment, the MerTK inhibitor and the immune checkpoint inhibitor are further combined with a Toll-like receptor (TLR) agonist and/or ionizing radiation.

In one embodiment, a compound of Formula I, as described herein, in combination with an immune checkpoint inhibitor, is provided for use in treating prostate cancer. In one embodiment, the immune checkpoint inhibitor is an anti-programmed cell death −1 (PD1) antibody. In one embodiment, the immune checkpoint inhibitor is an anti-CTLA4 antibody. In one embodiment, the MerTK inhibitor administered is selected from the compounds of Table 1. In one embodiment, the MerTK inhibitor administered is UNC2371. In one embodiment, UNC2371 is administered in combination with an anti-PD1 antibody. In one embodiment, UNC2371 is administered in combination with pembrolizumab. In one embodiment, UNC2371 is administered in combination with nivolumab. In one embodiment, UNC2371 is administered in combination with an anti-CTLA4 antibody. In one embodiment, UNC2371 is administered in combination with ipilimumab. In one embodiment, the MerTK inhibitor and the immune checkpoint inhibitor are further combined with a Toll-like receptor (TLR) agonist and/or ionizing radiation.

In one embodiment, a compound of Formula I, as described herein, in combination with an immune checkpoint inhibitor, is provided for use in treating a melanoma. In one embodiment, the immune checkpoint inhibitor is an anti-programmed cell death −1 (PD1) antibody. In one embodiment, the immune checkpoint inhibitor is an anti-CTLA4 antibody. In one embodiment, the host does not have a melanoma with a B-RAF mutation. In one embodiment, the host has a melanoma with a B-RAF mutation. In one embodiment, the host has a melanoma with a RAS mutation. In one embodiment, the melanoma over-expresses MerTK. In one embodiment, the melanoma has metastasized. In one embodiment, the MerTK inhibitor administered is selected from the compounds of Table 1. In one embodiment, the MerTK inhibitor administered is UNC2371. In one embodiment, UNC2371 is administered in combination with an anti-PD1 antibody. In one embodiment, UNC2371 is administered in combination with pembrolizumab. In one embodiment, UNC2371 is administered in combination with nivolumab. In one embodiment, UNC2371 is administered in combination with an anti-CTLA4 antibody. In one embodiment, UNC2371 is administered in combination with ipilimumab. In one embodiment, the MerTK inhibitor and the immune checkpoint inhibitor are further combined with a Toll-like receptor (TLR) agonist and/or ionizing radiation.

In one embodiment, a compound of Formula I, as described herein, in combination with an immune checkpoint inhibitor, is provided for use in treating Acute Lymphoblastic Leukemia (ALL). In one embodiment, a method is provided to treat a host with ALL comprising administering to the host an effective amount of a compound of Formula I in combination with an immune checkpoint inhibitor. In one embodiment, the MerTK inhibitor administered is selected from the compounds of Table 1. In one embodiment, the MerTK inhibitor administered is UNC2371. In one embodiment, the MerTK inhibitor and the immune checkpoint inhibitor are further combined with a Toll-like receptor (TLR) agonist and/or ionizing radiation.

In one embodiment, a compound of Formula I, as described herein, in combination with an immune checkpoint inhibitor, is provided for use in treating Acute Myeloid Leukemia (AML). In one embodiment, the AML contains a wild type FLT3 protein. In one embodiment, the replication of the AML cells are dependent on FLT3 expression. In one embodiment, the AML contains a FLT3-ITD mutation. In one embodiment, the AML contains a FLT3-TKD mutation. In one embodiment, the AML contains both a FLT3-ITD and FLT3-TKD mutation. In one embodiment, a FLT3 or dual MER/FLT3 inhibitor described herein is administered to a host suffering from AML, wherein the AML contains a mutation within the FLT3-TKD at amino acid F691 or D835. In one embodiment, the MerTK inhibitor administered is selected from the compounds of Table 1. In one embodiment, the MerTK inhibitor administered is UNC2371. In one embodiment, the MerTK inhibitor and the immune checkpoint inhibitor are further combined with a Toll-like receptor (TLR) agonist and/or ionizing radiation.

### Pharmaceutical Compositions and Dosages

The selected MerTK inhibitor and immune checkpoint inhibitors as described herein may be formulated individually, or if possible or desired, admixed, for administration in a pharmaceutical carrier to treat a host, typically a human, with any of the disorders described herein, in accordance with known techniques. See, e.g., Remington, The Science and Practice of Pharmacy (9^{th} Ed. 1995). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the active compound. One or more active compounds may be incorporated in the formulations of the invention, which may be prepared by any of the well-known techniques of pharmacy comprising admixing the components, optionally including one or more accessory ingredients.

In one aspect, the invention provides a pharmaceutical composition comprising a pharmaceutically effective amount of a MerTK inhibitor in combination with an immune checkpoint inhibitor as described herein and a pharmaceutically acceptable carrier. In another embodiment, the MerTK inhibitor is administered orally and the checkpoint inhibitor is administered via intravenous, intramuscular, subcutaneous, or other route known and suitable for a quickly degradable protein.

The MerTK inhibitor and immune complex inhibitor provided herein are administered in in physical or temporal combination for medical therapy in a therapeutically effective amount. The amount of the compounds administered will typically be determined by a physician, in the light of the relevant circumstances, including the condition to be treated, the chosen route of administration, the compound administered, the age, weight, and response of the individual patient, the severity of the patient's symptoms, and the like.

In one aspect of the present invention as described above, the MerTK inhibitor can be administered in combination with an immune checkpoint inhibitor, wherein the MerTK inhibitor dose and/or the immune checkpoint inhibitor dose is a subtherapeutic dose for the disorder being treated.

The MerTK inhibitor and/or immune checkpoint inhibitor can be administered by any suitable route associated with the particular compound, for example, the compounds may be administered by orally, rectally, buccally (e.g., sub-lingual), vaginally, parenterally (e.g., subcutaneous, intramuscular, intradermal, or intravenous), topically (i.e., both skin and mucosal surfaces, including airway surfaces), transdermally, intraventricular injection (injection into a ventricle of the brain, e.g., by an implanted catheter or Ommaya reservoir, such as in the case of morbid obesity), ocularly (via injection, implantation or by reservoir), and intranasally, although the most suitable route in any given case will depend on the nature and severity of the condition being treated and on the nature of the particular active compound which is being used.

The therapeutically effective dosage of any active compound described herein will be determined by the health care practitioner depending on the condition, size and age of the patient as well as the route of delivery. In one non-limited embodiment, a dosage from about 0.1 to about 200 mg/kg of the MerTK inhibitor and immune checkpoint inhibitor, independently, is herein contemplated, with all weights being calculated based upon the weight of the active compound, including the cases where a salt is employed. In some embodiments, the dosage can be the amount of compound needed to provide a serum concentration of the MerTK inhibitor and immune checkpoint inhibitor of up to between about 1 and 5, 10, 20, 30, or 40 μM.

In one aspect of the invention, a method is provided to treat a host suffering from a cancer by administering a daily amount of a MerTK inhibitor in combination with an immune checkpoint inhibitor, wherein the MerTK inhibitor is administered in a dose between about 0.5 mg and about 200 mg per administration, which may be at least 1, 2, 3, 4, or 5 times a day or perhaps only periodically on certain days, as instructed by the attending physician. In one embodiment, the MerTK inhibitor dose is at least about 1 mg, about 2 mg, about 3 mg, about 4 mg, about 5 mg, about 10 mg, about 12 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 125 mg, about 140 mg, about 150, about 175, or about 200 mg. In another embodiment, the MerTK inhibitor dose is between about 200 mg and 1250 mg. In one embodiment, the MerTK inhibitor dose is about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg or more.

In one embodiment, a compound of Formula I is combined for therapy temporally or physically with an additional anti-tumor agent, anti-neoplastic agent, anti-cancer agent, immunomodulatory agent, or immunostimulatory agent in addition to the use of the immune checkpoint inhibitor. The dosage administered to the host can be similar to that as administered during monotherapy treatment, or may be lower, for example, between about 0.5 mg and about 150 mg. In one embodiment, the dose is at least about 1 mg, about 2 mg, about 3 mg, about 4 mg, about 5 mg, about 10 mg, about 12 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 125 mg, about 140 mg, or about 150 mg.

In one embodiment, the invention provides a pharmaceutically acceptable composition for use as a chemotherapeutic comprising a compound of Formula I, or a salt, isotopic analog, prodrug, or a combination thereof, and an immune checkpoint inhibitor. In another embodiment, a compound of Formula I is administered orally and is provided in combination with an immune checkpoint inhibitor administered intravenously. In one embodiment, the immune checkpoint inhibitor, for example an anti-PD1, anti-PD-L1, or anti-CTLA4 antibody, can be administered in a first pharmaceutical composition as an intravenous infusion, and a second pharmaceutical composition comprising one or more therapeutic agents, including a compound of Formula I, can be administered concurrently, prior to, or following administration of an immune checkpoint inhibitor, wherein the second pharmaceutical composition can be administered orally, intravenously, or subcutaneously.

The person of ordinary skill will realize that methods of determining effective dosages of the selected immune checkpoint inhibitor, such as an antibody, to administer to a patient in need thereof, either alone or in combination with one or more other agents, may be determined by standard dose-response and toxicity studies that are well known in the art. In one embodiment, an immune checkpoint inhibitor such as an antibody may be administered at about 0.3-10 mg/kg, or the maximum tolerated dose, administered periodically, according to the judgement of the physician. Nonlimiting examples of dosage regimens include up to about every week, about every two weeks, three weeks, every six weeks, or about every three months. Alternatively, the immune checkpoint inhibitor antibody may be administered by an escalating dosage regimen including administering a first dosage at about 3 mg/kg, a second dosage at up to about 5 mg/kg, and a third dosage at about 9 mg/kg. Alternatively, the escalating dosage regimen includes administering a first dosage of immune checkpoint inhibitor antibody at up to about 5 mg/kg and a second dosage at up to about 9 mg/kg. Another stepwise escalating dosage regimen may include administering a first dosage of immune checkpoint inhibitor antibody up to about 3 mg/kg, a second dosage of up to about 3 mg/kg, a third dosage of up to about 5 mg/kg, a fourth dosage of up to about 5 mg/kg, and a fifth dosage of up to about 9 mg/kg. In another aspect, a stepwise escalating dosage regimen may include administering a first dosage of up to 5 mg/kg, a second dosage of up to 5 mg/kg, and a third dosage of up to 9 mg/kg.

Non-limiting examples of suitable dosages of an immune checkpoint inhibitor antibody include 3 mg/kg ipilimumab administered intravenously over 90 minutes every three weeks for four doses; 10 mg/kg ipilimumab every three weeks for eight cycles; 10 ipilimumab mg/kg every three weeks for four cycles then every 12 weeks for a total of three years; 2 mg/kg pembrolizumab administered intravenously over 30 minutes every three weeks; 10 mg/kg pembrolizumab every two or every three weeks; 15 mg/kg tremilimumab every three months; between 6-15 mg/kg tremilimumab every three months; 3 mg/kg nivolumab administered intravenously over 60 minutes every two weeks; between 0.3-10 mg/kg nivolumab every two weeks; 0.1, 0.3, 1, 3 or 10 mg/kg nivolumab every two weeks for up to 96 weeks.

### EXAMPLES

The present invention is illustrated in the following non-limiting Examples.

### Example 1

### Syntheses of MerTK Inhibitors

Scheme 1 illustrates a general procedure for preparing a compound of the present invention. Structure 4-1 can be prepared by alkylating a desired 7H-pyrrolo[2,3-d]pyrimidine with a desired cyclohexanol compound according to methods known in the art. The hydroxyl group on the cyclohexanol compound can be protected and deprotected by one skilled in the art to generate compounds of Formula I. For example, in one embodiment, the cyclohexanol compound can be protected with a tert-butyldimethylsilyl protecting group. See, for example, Greene, T. W. and Wuts, P. G. M., Protective Groups in Organic Synthesis, 2^{th} Ed., New York, John Wiley and Sons, Inc., 1991. Structure 4-1 can be prepared by treating a desired 7H-pyrrolo[2,3-d]pyrimidine, for example, 5-bromo-2-chloro-7H-pyrrolo[2,3-d]pyrimidine with a desired alcohol, for example, cis-4-(tert-butyldimethylsilyloxy)cyclohexanol in the presence of a phosphorane, for example, (cyanomethylene)trimethylphosphorane (CMMP; prepared according to Chem. Pharm. Bull. 2003, 51(4), 474-476.) in the presence of an organic solvent, for example, toluene at an elevated temperature. In one embodiment, LG₁ is a leaving group. In one embodiment, LG₁ is chloride. In one embodiment, LG₂ is a leaving group. In one embodiment, LG₂ is bromide. Structure 4-2 can be prepared by aminating a desired 7H-pyrrolo[2,3-d]pyrimidine, Structure 4-1, with a desired amine in an organic solvent, for example, 2-propanol, optionally at an elevated temperature in a microwave apparatus. For example, a desired 7H-pyrrolo[2,3-d]pyrimidine can be treated with a desired amine, for example, n-butylamine, in an organic solvent, for example, 2-propanol, at an elevated temperature in a microwave apparatus. In one embodiment, the desired amine can comprise a hydroxyl group that can optionally be protected with a protecting group, for example, a tert-butyldimethylsilyl protecting group. In Step 3, Structure 4-3 can be prepared by treating a desired 7H-pyrrolo[2,3-d]pyrimidine with a desired aryl compound according to methods known in the art.

The preparation of aryl boronic acid esters are well known to those skilled in the art. See, for example, “An Improved System for the Palladium-Catalyzed Borylation of Aryl Halides and Pinacol Borane”, K. L. Billingsley and S. L. Buchwald, J. Org. Chem., 2008, 73, 5589-5591. For example, Structure 4-3 can be prepared by treating Structure 4-2 with a desired boronic acid ester, for example, 4-(4-methylhomopiperazino)methylphenylboronic acid pinacol ester, an organometallic reagent, for example, tetrakis(triphenylphosphine)palladium(0), a base, for example, potassium carbonate, and a mixture of solvents optionally in a microwave apparatus optionally at an elevated temperature of about 100° C. In one embodiment, the mixture of solvents comprises dioxane and water. In one embodiment, the aryl compound can be protected with a protecting group that can be removed later in the synthesis. See, for example, Greene, T. W. and Wuts, P. G. M., Protective Groups in Organic Synthesis, 2^{th} Ed., New York, John Wiley and Sons, Inc., 1991. A compound of Formula I can be prepared by treating Structure 4-3 with an acid and an organic solvent to remove a tert-butyldimethylsilyl protecting group(s). In one embodiment, the acid is 1% concentrated hydrochloric acid. In one embodiment, the organic solvent is methanol. R¹ is as defined herein. The preparation of pyrrolopyrimidine compounds is disclosed in WO 2013/052417 to Wang et al. incorporated herein by reference. This chemistry is illustrated in Scheme 1.

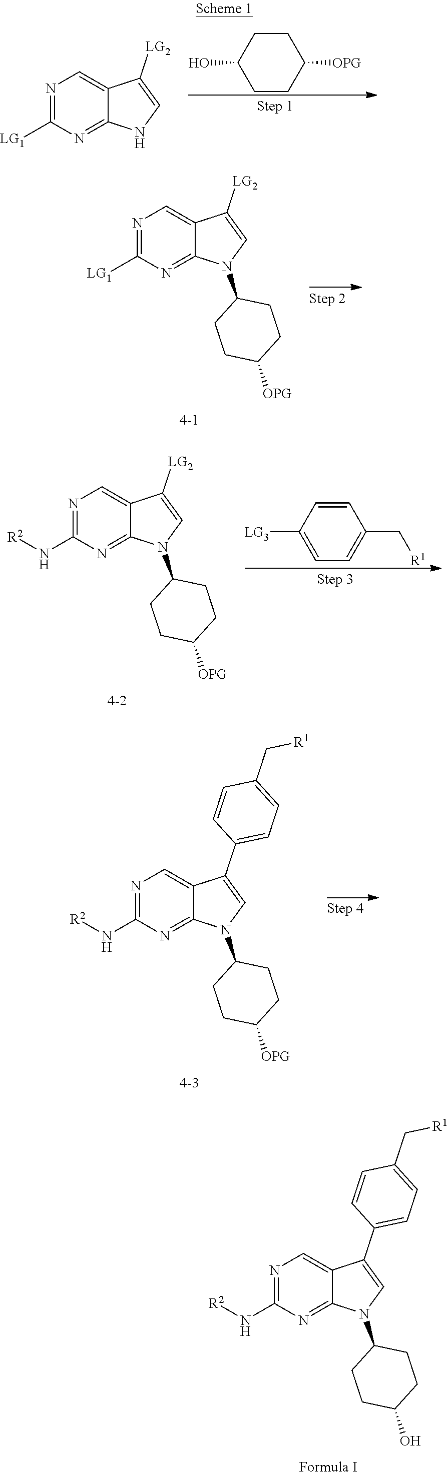

Compounds of Formula I can be metabolized to generate pyrrolo-pyrimidine compounds. In one embodiment, a compound of Formula I can be dealkylated. For example, a compound of Formula I can be dealkylated, i.e., removal of R², to generate a free amino group on the pyrrolopyrimidine. In one embodiment, a compound of Formula I comprising a methylated amine can be demethylated. In one embodiment, a compound of Formula I can be oxidized. For example, a compound of Formula I comprising a piperazine group can be oxidized to generate a piperazine N-oxide. In another embodiment, a compound of Formula I comprising a piperazine group can be oxidized twice to generate a piperazine bis-N-oxide. In another embodiment, a compound of Formula I can be oxidized to generate a pyrrolopyrimidine N-oxide. The metabolic pathways described above are illustrated below, in Scheme 2, with the example compound UNC2371.

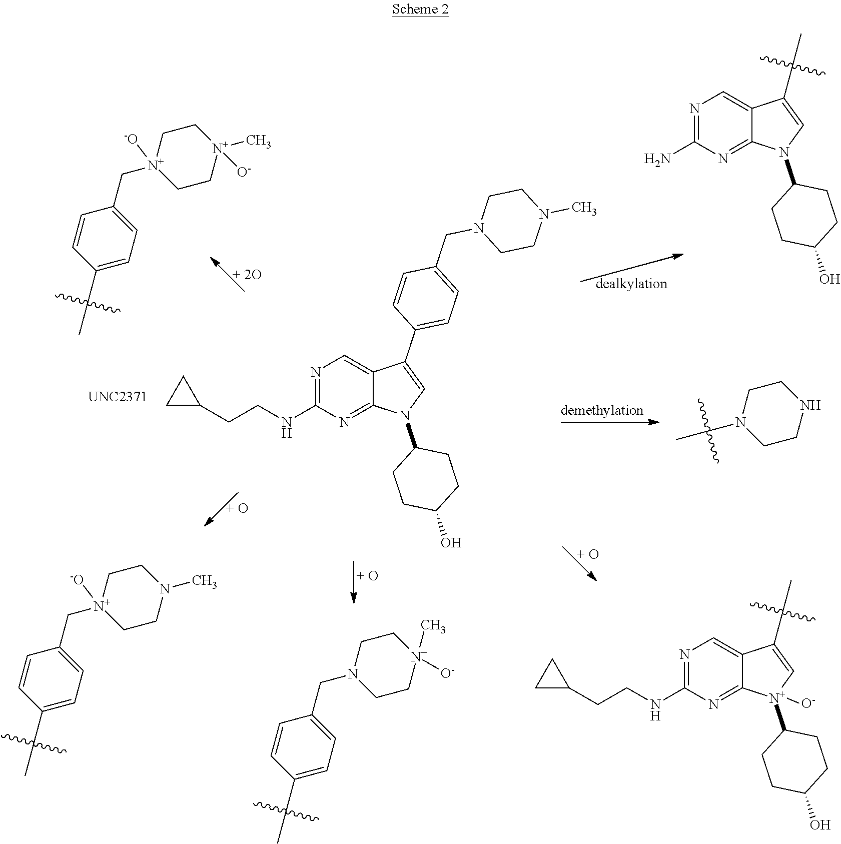

### Example 2

### Syntheses of Pyrrolopyrimidine Compounds

Trans-4-(2-(Butylamino)-5-(4-fluorophenyl)-7H-pyrrolo[2,3-d]pyrimidin-7-yl)cyclohexanol

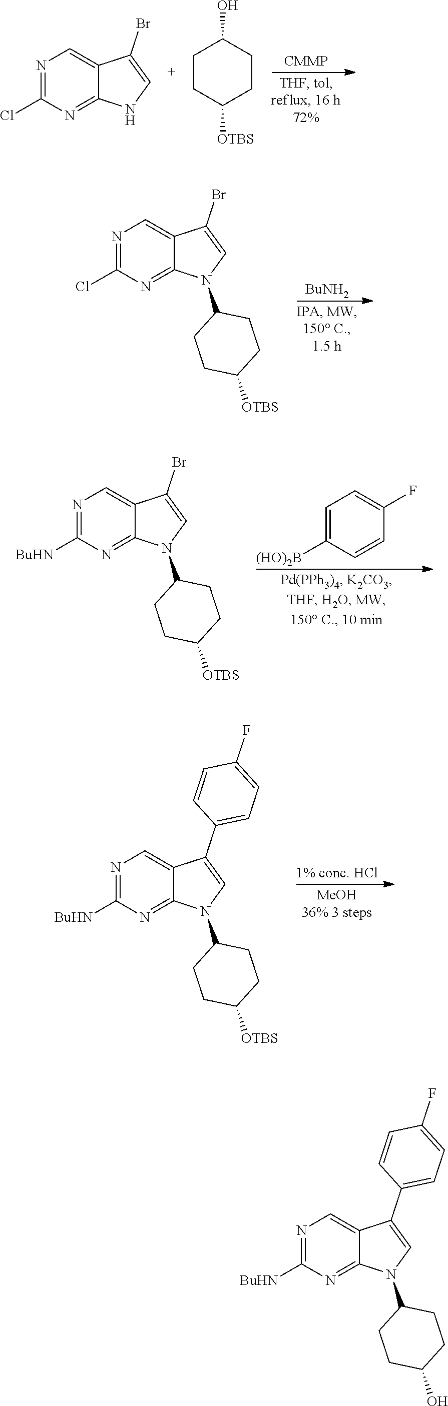

5-Bromo-7-(trans-4-((tert-butyldimethylsilyl)oxy)cyclohexyl)-2-chloro-7H-pyrrolo[2,3-d]pyrimidine

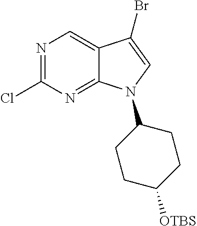

To a suspension of 5-bromo-2-chloro-7H-pyrrolo[2,3-d]pyrimidine (0.13 g, 0.50 mmol) and cis-4-(tert-butyldimethylsilyloxy)cyclohexanol (0.23 g, 1.0 mmol) in toluene (8 mL) was added (cyanomethylene)trimethylphosphorane (CMMP; prepared according to Chem. Pharm. Bull. 2003, 51(4), 474-476.) (6.3 mL, 0.16 M in THF, 1.0 mmol). The resulting clear solution was refluxed for 16 h. The reaction mixture was washed with brine, and extracted with EtOAc (3×). The combined organic layer was dried (Na₂SO₄) and concentrated. The residue was purified on ISCO to provide the desired product (0.16 g, 72%). 1H NMR (400 MHz, CD₃OD) δ8.71 (s, 1H), 7.27 (s, 1H), 4.70 (tt, J=12.2, 3.9 Hz, 1H), 3.69 (tt, J=10.5, 4.2 Hz, 1H), 2.09-1.99 (m, 3H), 1.86-1.71 (m, 2H), 1.66-1.54 (m, 3H), 0.90 (s, 9H), 0.08 (s, 6H). MS m/z 444.2 [M+H]⁺.

Trans-4-(2-(butylamino)-5-(4-fluorophenyl)-7H-pyrrolo[2,3-d]pyrimidin-7-yl)cyclohexanol

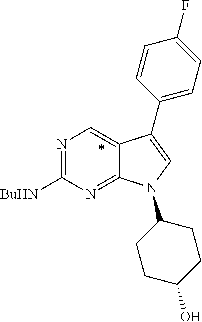

To a solution of 5-bromo-7-(trans-4-(tert-butyldimethylsilyloxy)cyclohexyl)-2-chloro-7H-pyrrolo[2,3-d]pyrimidine (0.082g, 0.18 mmol) in isopropyl alcohol (2.0 mL) was added n-butylamine (0.033 g, 0.45 mmol) in a microwave tube. The resulting mixture was heated under microwave irradiation at 150° C. for 1.5 h. After the reaction cooled to room temperature, the solvent and excess amine was evaporated under vacuum. The residue was dissolved in THF and concentrated under vacuum (3×). Then it was dissolved in THF (2.0 mL) in a microwave tube. To this solution was added K₂CO₃ (0.050 g, 0.36 mmol), Pd(PPh₃)₄ (0.021 g, 0.018 mmol), (4-fluorophenyl)boronic acid (0.038 g, 0.27 mmol), and H₂O (0.5 mL). The resulting mixture was heated under microwave irradiation at 150° C. for 10 min. After cooled to room temperature, it was washed with brine and extracted with EtOAc (5×). The combined organic layer was dried (Na₂SO₄) and concentrated. The residue was filtered through a short column of silica gel to provide N-butyl-7-(trans-4-((tert-butyldimethylsilyl)oxy) cyclohexyl)-5-(4-fluorophenyl)-7H-pyrrolo[2,3-d]pyrimidin-2-amine which was used for next step without further purification.

A solution of crude N-butyl-7-(trans-4-((tert-butyldimethylsilyl)oxy) cyclohexyl)-5-(4-fluorophenyl)-7H-pyrrolo[2,3-d]pyrimidin-2-amine in MeOH (2.0 mL) was added a concentrated HCl solution (3 drops, 37% in water). The resulting solution was stirred at room temperature overnight, then concentrated. The residue was purified by pre-HPLC to provide the desired product (UNC1671A) (0.025 g, 36% over 3 steps). 1HNMR (400 MHz, CD₃OD) δ8.73 (s, 1H), 7.80 (s, 1H), 7.69-7.62 (m, 2H), 7.24-7.16 (m, 2H), 4.64-4.52 (m, 1H), 3.79-3.67 (m, 1H), 3.55 (t, J=7.1 Hz, 2H), 2.18-2.11 (m, 2H), 2.11-2.01 (m, 4H), 1.77-1.66 (m, 2H), 1.59-1.44 (m, 4H), 1.03 (t, J=7.4 Hz, 3H); MS m/z 383.2 [M+H]⁺.

trans-4-(2-(butylamino)-5-(4-((4-methyl-1,4-diazepan-1-yl)methyl)phenyl)-7H-pyrrolo[2,3-d]pyrimidin-7-yl)cyclohexan-1-ol

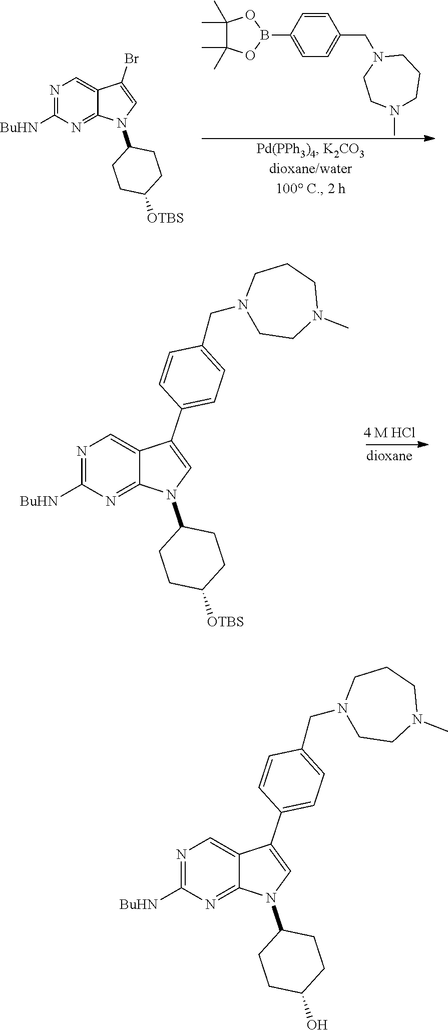

A solution of 5-bromo-N-butyl-7-(trans-4-((tert-butyldimethylsilyl)oxy)cyclohexyl)-7H-pyrrolo[2,3-d]pyrimidin-2-amine (120 mg, 0.25 mmol) and 4-(4-methylhomopiperazino)methylphenylboronic acid pinacol ester (124 mg, 0.38 mmol) in a mixture of dioxane and water (4:1, 1 ml) was added tetrakis(triphenylphosphine)palladium(0) (15 mg, 0.012 mmol,) and K₂CO₃ (69 mg, 0.5 mmol). The resulting mixture was stirred at 100° C. for 12 h. The reaction was quenched with water (3 ml) and the aqueous layer was extracted with EtOAc (3 ml×3). The combined organic layer was washed with brine (5 ml), dried (Na₂SO₄), and concentrated to afford N-butyl-7-(trans-4-((tert-butyldimethylsilyl)oxy)cyclohexyl)-5-(4-(4-methyl-1,4-diazepan-l-yOmethyl)phenyl)-7H- pyrrolo[2,3-d]pyrimidin-2-amine (MS m/z 604.40 [M+H]⁺). The crude product was treated with 4 N HCl in dioxane and concentrated. The residue was was purified on HPLC to provide the title compound as a TFA salt, which was converted to HCl salt by the treatment of a 4.0 N HCl solution in dioxane and lyophilized to yield the title compound (UNC4218A) (12 mg, 10% over 2 steps). ¹H NMR (400 MHz, cd₃od) δ8.82 (s, 1H), 7.96 (s, 1H), 7.81-7.73 (m, 4H), 4.65-4.51 (m, 3H), 3.86-3.69 (m, 6H), 3.55-3.53 (m, 4H), 2.99 (s, 3H), 2.38 (br, 2H), 2.10-2.06 (m, 6H), 1.72-1.69 (m, 3H), 1.52-1.45 (m, 4H), 1.03 (t, J =8.0 Hz, 3H);MS m/z 491.40 [M+1]⁺.

Table 1 describes compounds prepared following procedures described in Examples 1 and 2 using appropriate reagents. (Note: MerTK IC50: ++++ means <10 nM; +++ means between 10-100nM, ++ means between 100 nM-1 μM; + means between 1-30 μM; − means inactive.). The compounds of Table 1 have been previously disclosed in WO 2013/052417 (UNC2025A, UNC2142A, UNC2143A, UNC2370A, UNC2371, UNC2395A, and UNC2396A) and PCT/US2015/024381 (UNC4218A and UNC3908A).

| | | | | Physical Data |
|---|---|---|---|---|
| | | | | MS m/z (M + 1) |
| | | | Mer | or/and ¹H NMR |
| | Structure | Compound_ID | IC₅₀ | (400 MHz, CD₃OD) |
| 1 | 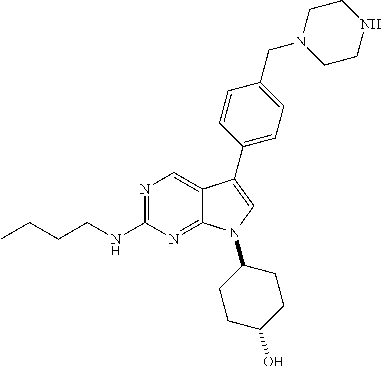 | UNC2025A | ++++ | ¹H NMR (400 MHz, CD₃OD) δ 8.83 (s, 1H), 7.96 (s, 1H), 7.80 (d, J = 8.3 Hz, 2H), 7.74 (d, J = 8.4 Hz, 2H), 4.66-4.56 (m, 1H), 4.53 (s, 2H), 3.91-3.58 (m, 9H), 3.55 (t, J = 7.1 Hz, 2H), 3.02 (s, 3H), 2.19-2.11 (m, 2H), 2.11-1.99 (m, 4H), 1.78-1.66 (m, 2H), 1.58- 1.41 (m, 4H), 1.02 (t, J = 7.4 Hz, 3H); ¹³C NMR (101 MHz, CD₃OD) δ 154.6, 151.1, 138.7, 134.0, 132.1, 127.2, 127.0, 116.7, 110.0, 109.9, 68.5, 53.9, 50.0, 40.9, 33.7, 30.6, 29.5, 19.6, 12.7; MS m/z 477.35 [M + H]⁺. |
| 2 | 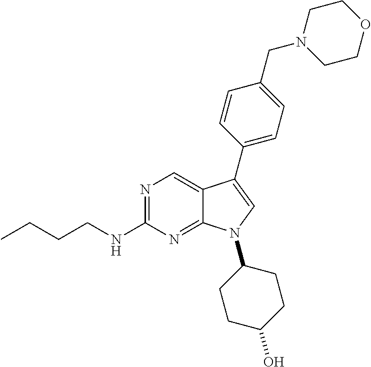 | UNC2142A | ++++ | ¹H NMR (400 MHz, CD₃OD) δ 8.81 (s, 1H), 7.95 (s, 1H), 7.83-7.77 (m, 2H), 7.69-7.63 (m, 2H), 4.66-4.57 (m, 1H), 4.41 (s, 2H), 4.05 (d, J = 12.7 Hz, 2H), 3.84-3.69 (m, 3H), 3.55 (t, J = 7.1 Hz, 2H), 3.44-3.36 (m, 2H), 3.28-3.18 (m, 2H), 2.18-2.11 (m, 2H), 2.11-2.01 (m, 4H), 1.77-1.68 (m, 2H), 1.57-1.44 (m, 4H), 1.03 (t, J = 7.4 Hz, 3H); MS m/z 464.30 [M + H]⁺. |
| 3 | 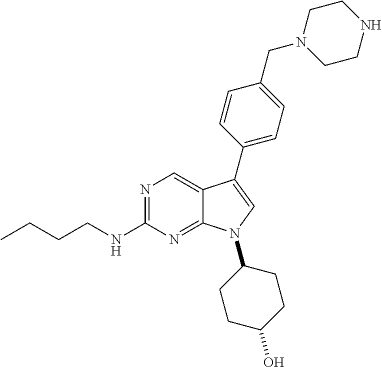 | UNC2143A | ++++ | ¹H NMR (400 MHz, CD₃OD) δ 8.81 (s, 1H), 7.95 (s, 1H), 7.82-7.76 (m, 2H), 7.75-7.69 (m, 2H), 4.65-4.57 (m, 1H), 4.48 (s, 2H), 3.77-3.69 (m, 1H), 3.66-3.50 (m, 10H), 2.20-2.03 (m, 6H), 1.77-1.67 (m, 2H), 1.58-1.45 (m, 4H), 1.03 (t, J = 7.4 Hz, 3H); MS m/z 463.30 [M + H]⁺. |
| 4 | 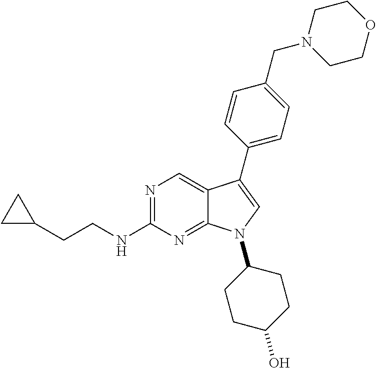 | UNC2370A | ++++ | ¹H NMR (400 MHz, cd3od) δ 8.67 (s, 1H), 7.79 (s, 1H), 7.67-7.60 (m, 2H), 7.52-7.46 (m, 2H), 4.52-4.42 (m, 1H), 4.26 (s, 2H), 3.97-3.85 (m, 2H), 3.71-3.55 (m, 3H), 3.54-3.45 (m, 2H), 3.33-3.20 (m, 2H),3.14- 3.01 (m, 2H), 2.06-1.98 (m, 2H), 1.97-1.84 (m, 4H), 1.53-1.45 (m, 2H), 1.45-1.33 (m, 2H), 0.74- 0.62 (m, 1H), 0.42-0.33 (m, 2H), 0.06-0.03 (m, 2H); MS m/z 476.30 [M + H]⁺. |
| 5 | 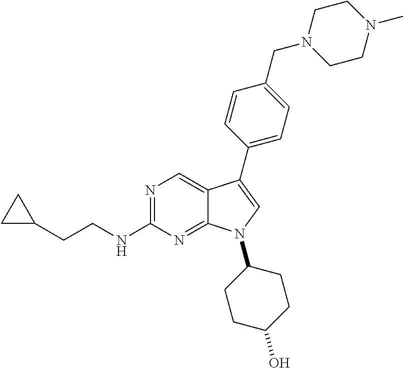 | UNC2371 | ++++ | ¹H NMR (400 MHz, cd3od) δ 8.58 (s, 1H), 7.47 (d, J = 8.2 Hz, 2H), 7.28-7.21 (m, 3H), 4.48- 4.36 (m, 1H), 3.66-3.53 (m, 1H), 3.47-3.37 (m, 4H), 2.53-2.29 (m, 6H), 2.19 (s, 3H), 2.06-1.97 (m, 2H), 1.96-1.81 (m, 4H), 1.50-1.34 (m, 4H), 1.23-1.09 (m, 1H), 0.90- 0.63 (m, 2H), 0.42-0.34 (m, 2H), 0.06-0.03 (m, 2H); MS m/z 489.40 [M + H]⁺. |
| 6 | 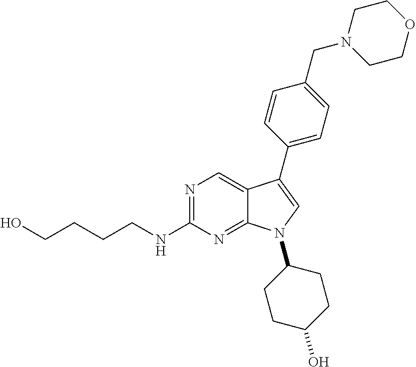 | UNC2395A | ++++ | ¹H NMR (400 MHz, cd3od) δ 8.80 (s, 1H), 7.93 (s, 1H), 7.81-7.74 (m, 2H), 7.62 (d, J = 8.3 Hz, 2H), 4.68-4.56 (m, 1H), 4.40 (s, 2H), 4.11- 3.95 (m, 2H), 3.83-3.68 (m, 3H), 3.68-3.54 (m, 4H), 3.50-3.35 (m, 2H), 3.29-3.16 (m, 2H), 2.20- 1.99 (m, 7H), 1.88-1.76 (m, 2H), 1.74-1.63 (m, 2H), 1.60-1.45 (m, 2H); MS m/z 480.30 [M + H]⁺. |
| 7 | 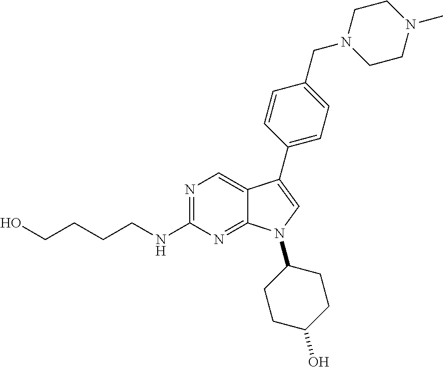 | UNC2396A | ++++ | ¹H NMR (400 MHz, cd3od) δ 8.77 (s, 1H), 7.88 (d, J = 4.2 Hz, 1H), 7.71 (d, J = 8.3 Hz, 2H), 7.56 (d, J = 8.3 Hz, 2H), 4.65-4.56 (m, 1H), 4.16 (s, 2H), 3.79-3.67 (m, 1H), 3.67-3.62 (m, 2H), 3.62-3.55 (m, 2H), 3.50 (s, 4H), 3.29-3.24 (m, 1H), 2.93 (s, 3H), 2.26- 1.91 (m, 7H), 1.86-1.73 (m, 2H), 1.73-1.63 (m, 2H), 1.60-1.46 (m, 2H); MS m/z 493.40 [M + H]⁺. |
| 8 | 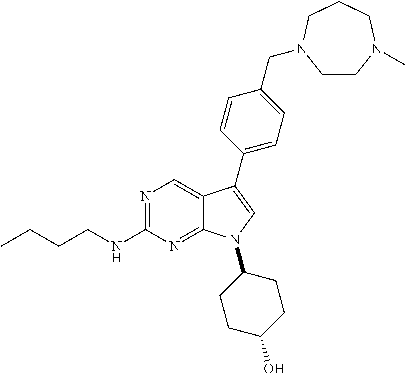 | UNC4218A | ++++ | ¹H NMR (400 MHz, cd3od) δ 8.82 (s, 1H), 7.96 (s, 1H), 7.81-7.73 (m, 4H), 4.6-54.51 (m, 3H), 3.86-3.69 (m, 6H), 3.55-3.53 (m, 4H), 2.99 (s, 3H), 2.38 (br, 2H), 2.10-2.06 (m, 6H), 1.72- 1.69 (m, 3H), 1.52-1.45 (m, 4H), 1.03 (t, J = 8.0 Hz, 3H); MS m/z 491.40 [M + H]⁺. |
| 9 | 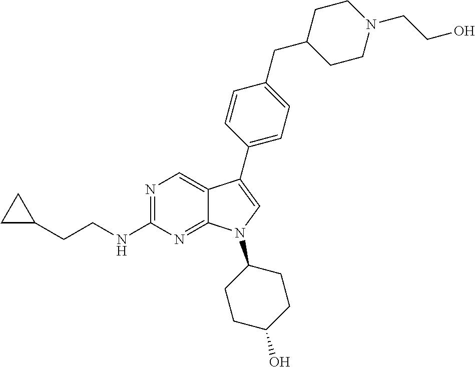 [SMILES: OCCN1CCC(Cc2ccc(-c3cn([C@H]4CC[C@H](O)CC4)c4nc(NCCC5CC5)ncc34)cc2)CC1] | UNC3908A | ++++ | ¹H NMR (400 MHz, CD₃OD) δ 8.73 (s, 1H), 7.79 (s, 1H), 7.59 (d, J = 8.2 Hz, 2H), 7.31 (d, J = 8.2 Hz, 2H), 4.63-4.56 (m, 1H), 3.89-3.82 (m, 2H), 3.73 (ddd, J = 10.3, 7.9, 4.0 Hz, 2H), 3.68- 3.55 (m, 6H), 3.24-3.15 (m, 2H), 3.03-2.89 (m, 2H), 2.67 (d, J = 6.7 Hz, 2H), 2.18-2.11 (m, 2H), 2.10-2.01 (m, 4H), 1.98- 1.86 (m, 3H), 1.66-1.49 (m, 6H), 0.85-0.78 (m, 1H), 0.56-0.49 (m, 2H), 0.19-0.13 (m, 2H); MS m/z 518.15 [M + H]⁺. |

### Example 3

### MerTK Inhibitors Provide a Synergsitic Reduction in Tumor Volume in Combination with an Immune Checkpoint Inhibitor

The efficacy of a combination therapy using a MerTK inhibitor and immune checkpoint inhibitor was examined in a colon carcinoma animal model. In the following example, the MerTK inhibitor UNC2371 was examined in combination with a PD-1 immune checkpoint inhibitor and a CTLA4 immune checkpoint inhibitor.

Female BALB/c mice were injected with 5×10⁵ Colon26 tumor cells in 0% Matrigel sc in flank. The cell injection volume was 0.1 mL/mouse. Mice were between 7 to 10 weeks at the start of the experiment. When tumors reached an average size of 80-120 mm³, treatment was begun. Body weight was measured five times per week for the first two weeks and then twice per week to the end of the experiment. Tumor volume was measured by caliper measurement twice per week to the end of the experiment. Any individual animal with a single observation of greater than 30% body weight loss or three consecutive measurements of greater than 25% body weight loss were euthanized. Any group with a mean body weight loss of greater than 20% or greater than 10% mortality was stopped dosing. The group was not euthanized and recovery was allowed. Within a group with greater than 20% weight loss, individuals hitting the individual body weight loss endpoint were euthanized. The endpoint of the experiment was a tumor volume of 1000 mm³ or 45 days, whichever endpoint came first. When the endpoint was reached, the mice were euthanized.

UNC2371 was prepared in salt form and the dosing solution was prepared every day. UNC2371 was prepared in deionized water. All doses represent total substance dosed by weight of compound (including salt weight). The amount of active compound dosed was as follows: dose 1.08 mg/kg=1 mg/kg active compound; dose 5.38 mg/kg=5 mg/kg active compound; dose 16.13 mg/kg=15 mg/kg active compound. Dosing solution was prepared at 5 mL/kg (0.100 mL/20 g mouse) and volume was adjusted accordingly for body weight. Dosing solutions of anti-CTLA4 9H10 (BioXcell cat# BE0131) and anti-PD1 RMP1-14 (rat Ig—BioXcell cat# BE0146) were prepared in PBS every week, stored at 4° C., and protected from light. Dosing solution was prepared at 10 mL/kg (0.200 mL/20 g mouse) and volume was adjusted accordingly for body weight.

Mice were randomized into twelve different experimental groups. Group 1 received deionized water via oral gavage (p.o.) three times daily for twenty-one days, starting with two doses on Day 1 (tid×21 first day two doses) and PBS i.p. twice weekly for two weeks (biwk×2). Groups 2-4 received UNC2371 via oral gavage (p.o.) three times daily for twenty-one days, starting with two doses on Day 1 (tid×21 first day two doses). Dosing amounts for UNC2371 for Groups 2-4 were 1, 5 and 15 mg/kg, respectively. Group 5 received anti-PD1 mAb at a dosage of 5 mg/kg i.p. biwk×2. Group 6 received anti-CTLA4 mAb at a dosage of 5 mg/kg on Day 1 and at 2.5 mg/kg on Days 4 and 7. Groups 7-9 received UNC2371 (p.o. tid×21, first day two doses) at 1, 5 and 15 mg/kg, respectively, in combination with anti-PD1 (5 mg/kg i.p. biwk×2). Groups 10-12 received UNC2371 (p.o. tid×21, first day two doses) at 1, 5 and 15 mg/kg, respectively, in combination with anti-CTLA4 (5 mg/kg on Day 1 and at 2.5 mg/kg on Days 4 and 7). Table 2 summarizes the study groups and the treatment regimens.

TABLE 2
Treatment Regimens for a MerTK Inhibitor (UNC2371) in Combination with Anti-
PD1 or Anti-CTLA4

| | | Regimen 1 | | | | Regimen 2 | | | | Regimen 3 | | | |
|---|---|---|---|---|---|---|---|---|---|---|---|---|---|
| Gr. | N | Agent | mg/kg | Route | Schedule | Agent | mg/kg | Route | Schedule | Agent | mg/kg | Route | Schedule |
| 1 | 10 | DI Water | — | po | tid × 21 | PBS | — | ip | biwk × 2 | — | — | — | — |
| | | | | | first day 2 | | | | | | | | |
| | | | | | doses | | | | | | | | |
| 2 | 10 | UNC2371 | 1.08 | po | tid × 21 | — | — | — | — | — | — | — | — |
| | | | | | first day 2 | | | | | | | | |
| | | | | | doses | | | | | | | | |
| 3 | 10 | UNC2371 | 5.38 | po | tid × 21 | — | — | — | — | — | — | — | — |
| | | | | | first day 2 | | | | | | | | |
| | | | | | doses | | | | | | | | |
| 4 | 10 | UNC2371 | 16.13 | po | tid × 21 | — | — | — | — | — | — | — | — |
| | | | | | first day 2 | | | | | | | | |
| | | | | | doses | | | | | | | | |
| 5 | 10 | anti-PD1 | 5 | ip | biwk x 2 | — | — | — | — | — | — | — | — |
| | | RMP1-14 | | | | | | | | | | | |
| 6 | 10 | anti- | 5 | ip | day 1 | anti- | 2.5 | ip | days 4, 7 | — | — | — | — |
| | | CTLA4 | | | | CTLA4 | | | | | | | |
| | | 9H10 | | | | 9H10 | | | | | | | |
| 7 | 10 | UNC2371 | 1.08 | po | tid × 21 | anti-PD1 | 5 | ip | biwk × 2 | — | — | — | — |
| | | | | | first day 2 | RMP1- | | | | | | | |
| | | | | | doses | 14 | | | | | | | |
| 8 | 10 | UNC23713 | 5.38 | po | tid × 21 | anti-PD1 | 5 | ip | biwk × 2 | — | — | — | — |
| | | | | | first day 2 | RMP1- | | | | | | | |
| | | | | | doses | 14 | | | | | | | |
| 9 | 10 | UNC2371 | 16.13 | po | tid × 21 | anti-PD1 | 5 | ip | biwk × 2 | — | — | — | — |
| | | | | | first day 2 | RMP1- | | | | | | | |
| | | | | | doses | 14 | | | | | | | |
| 10 | 10 | UNC2371 | 1.08 | po | tid × 21 | anti- | 5 | ip | day 1 | anti- | 2.5 | ip | days 4, 7 |
| | | | | | first day 2 | CTLA4 | | | | CTLA4 | | | |
| | | | | | doses | 9H10 | | | | 9H10 | | | |
| 11 | 10 | UNC2371 | 5.38 | po | tid × 21 | anti- | 5 | ip | day 1 | anti- | 2.5 | ip | days 4, 7 |
| | | | | | first day 2 | CTLA4 | | | | CTLA4 | | | |
| | | | | | doses | 9H10 | | | | 9H10 | | | |
| 12 | 10 | UNC2371 | 16.13 | po | tid × 21 | anti- | 5 | ip | day 1 | anti- | 2.5 | ip | days 4, 7 |
| | | | | | first day 2 | CTLA4 | | | | CTLA4 | | | |
| | | | | | doses | 9H10 | | | | 9H10 | | | |

po = per os (oral gavage),
ip = intraperitoneal;
tid = three times per day;
biwk = twice per week

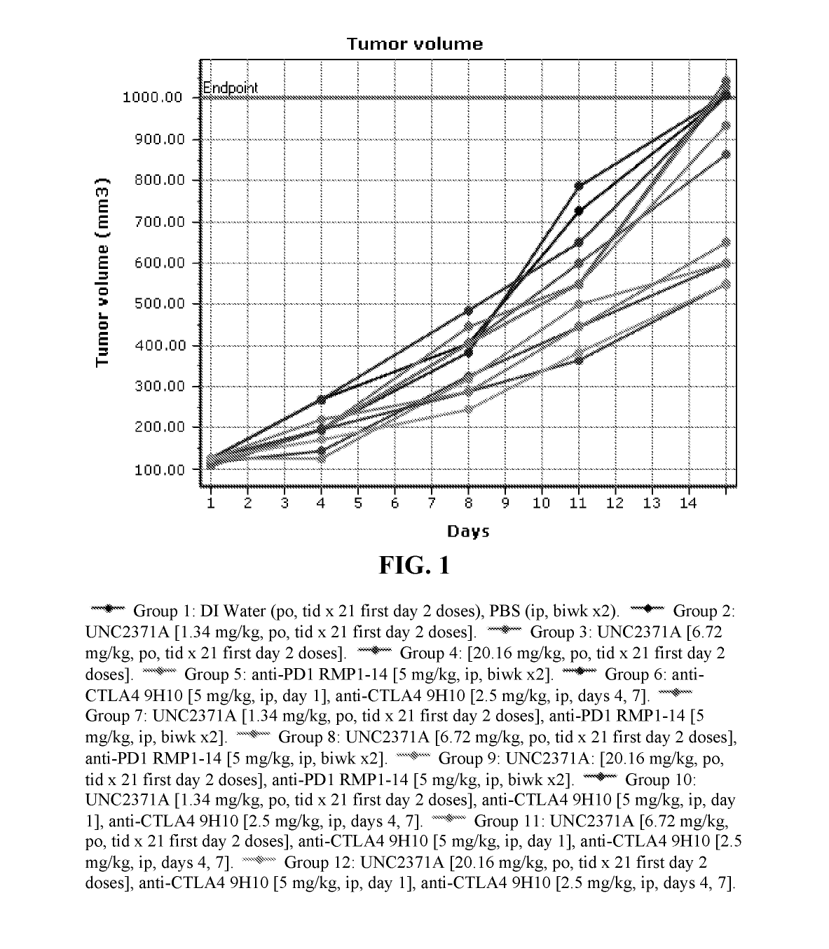

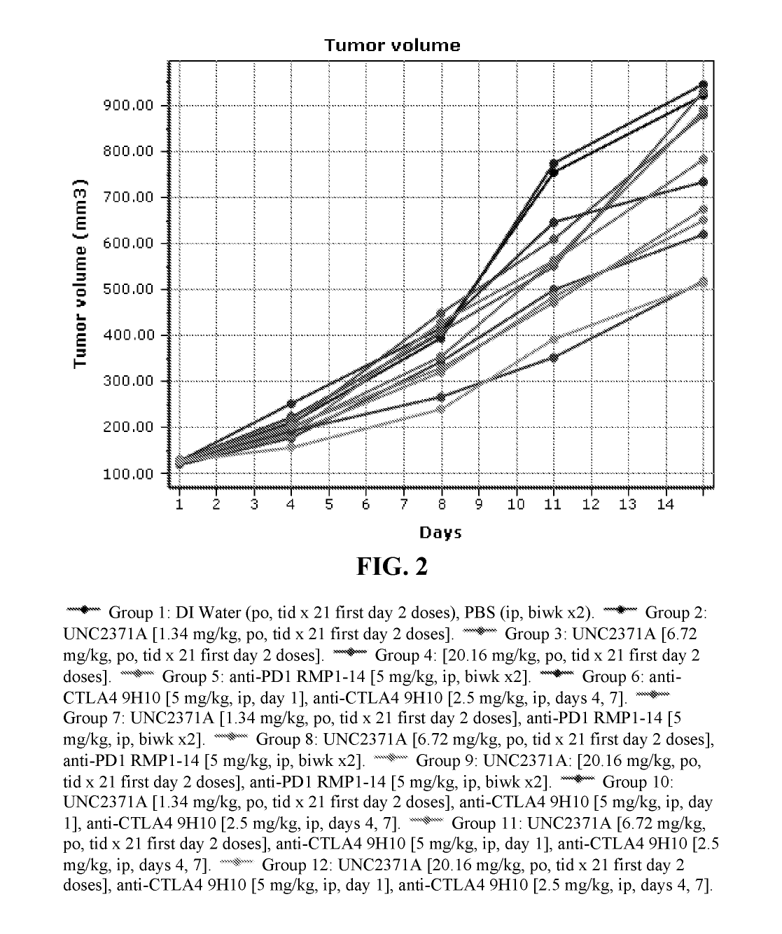

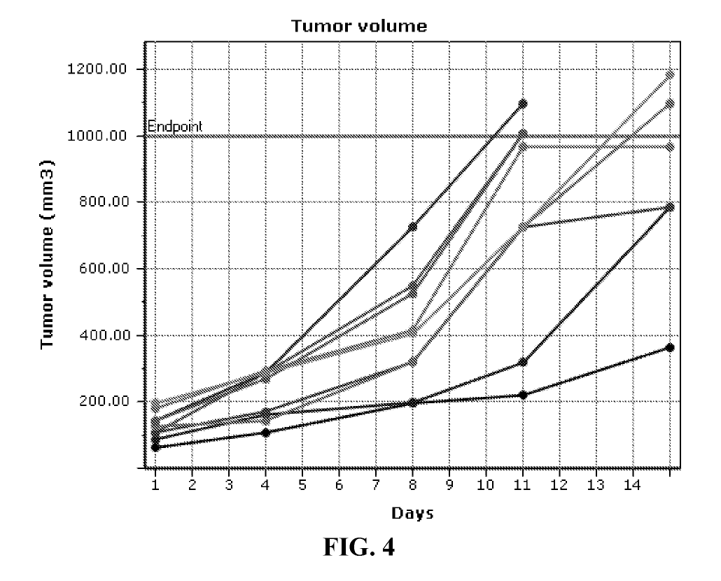

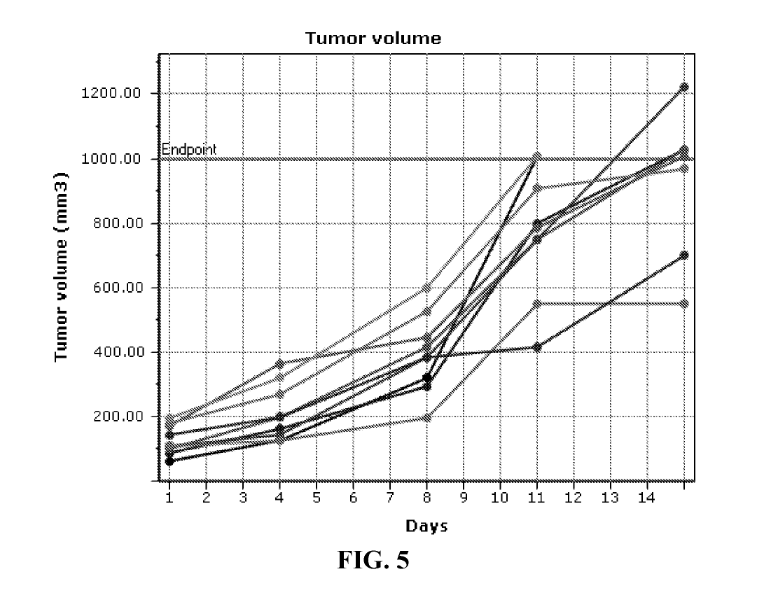

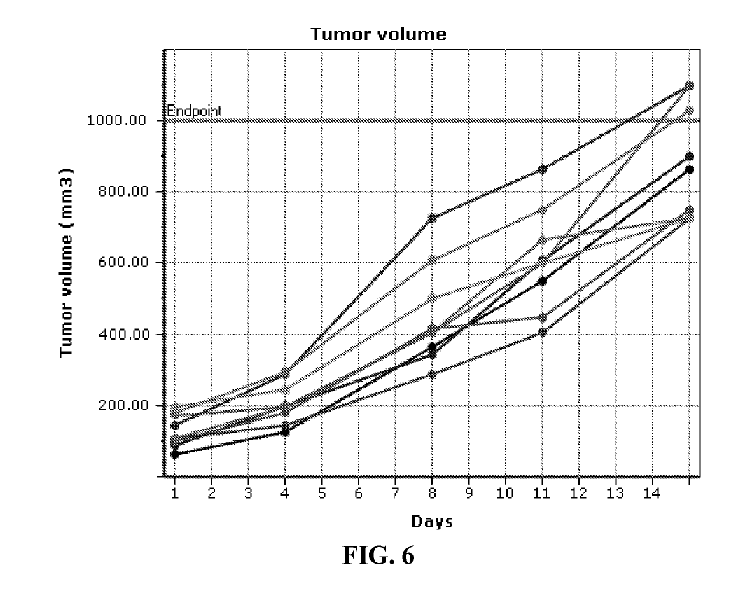

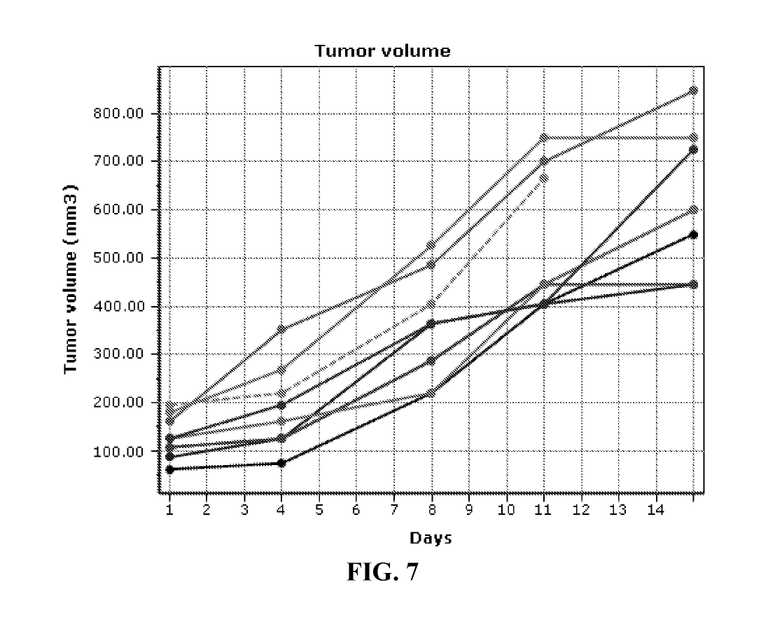

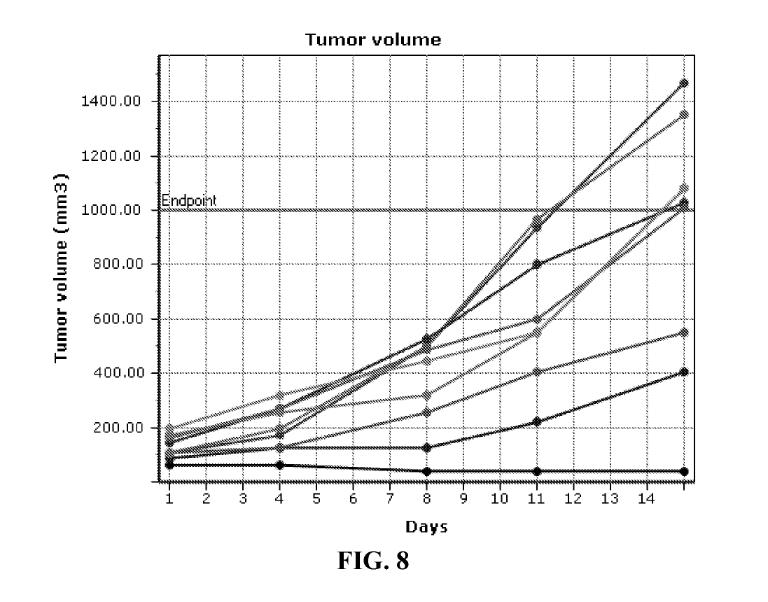

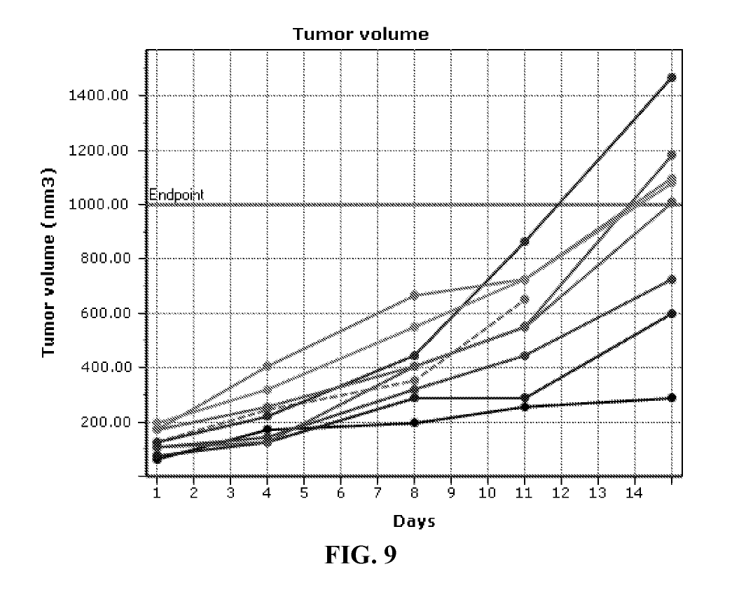

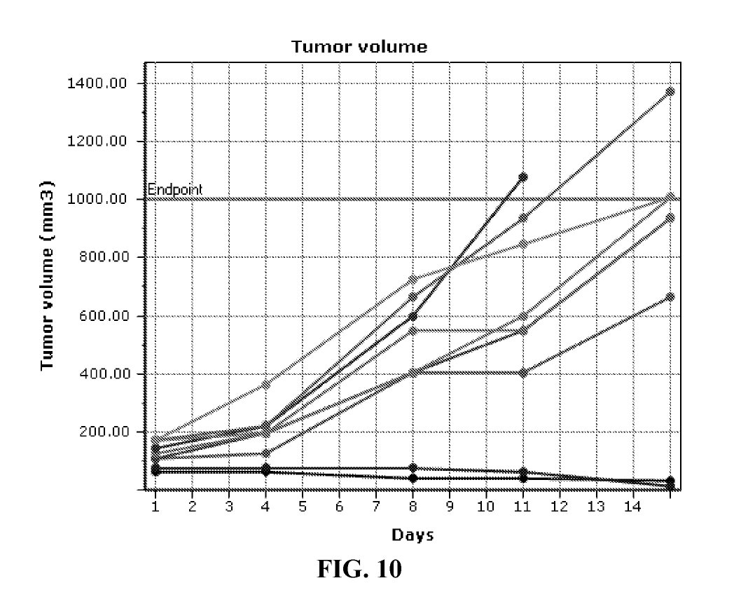

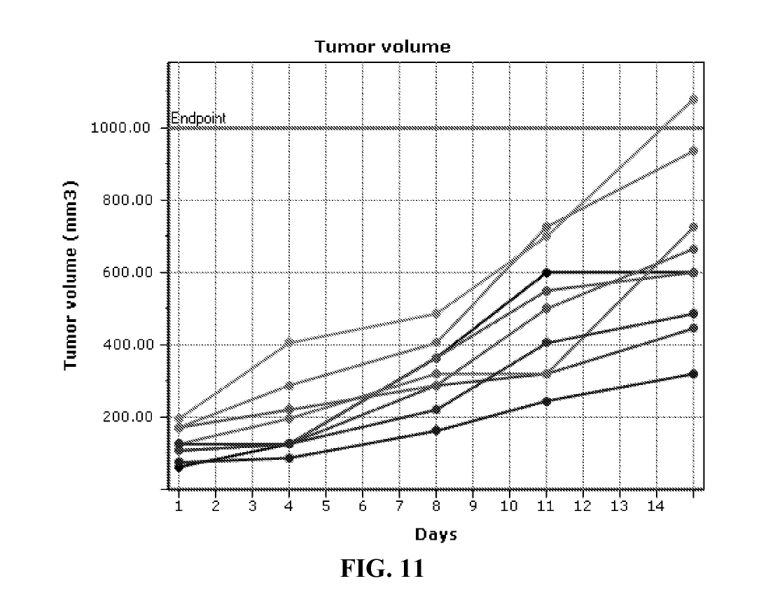

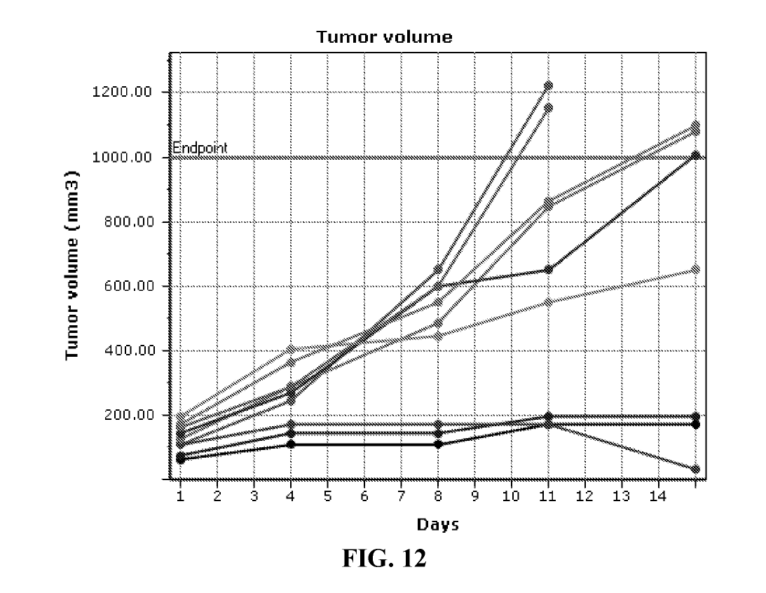

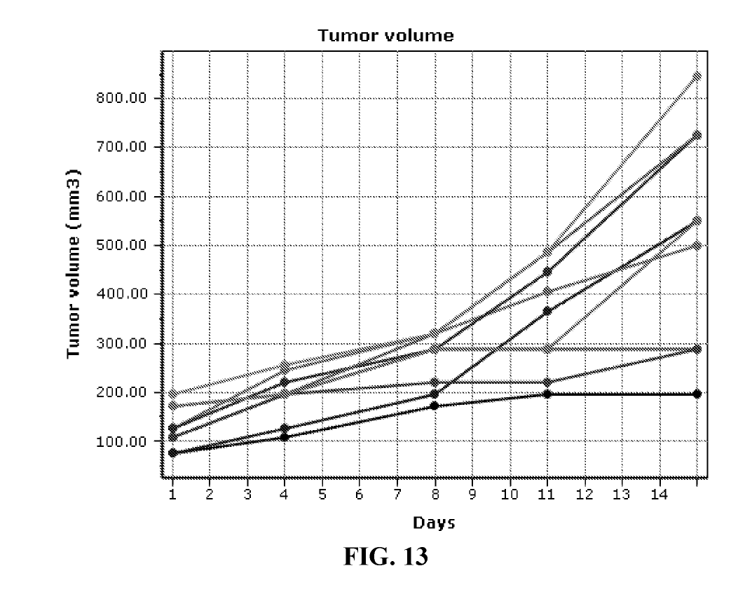

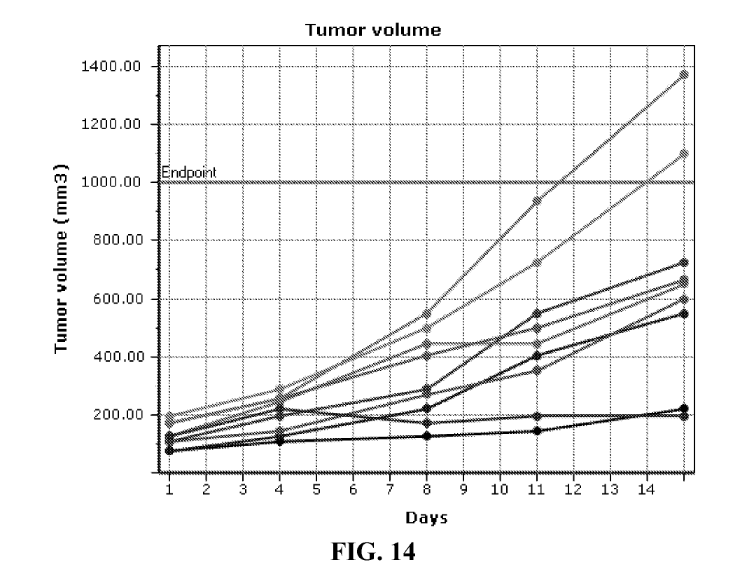

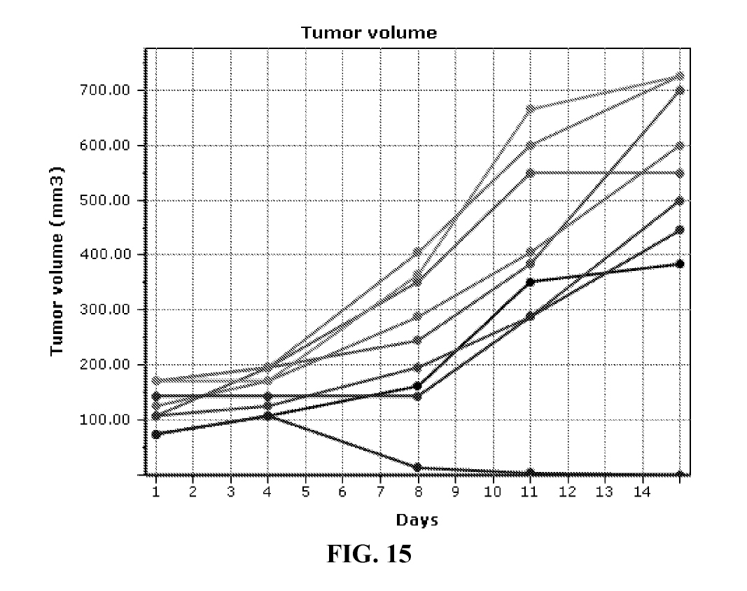

The results from each experimental group, for days 1 to 15, are shown in FIGS. 1 to 15. As seen in FIG. 1, the median tumor volume for mice treated with UNC2371 in combination with anti-CTLA4 was smaller than for mice treated individually with UNC2371 or anti-CTLA4. As seen in FIG. 2, the mean tumor volume for mice treated with UNC2371 in combination with anti-CTLA4 was also smaller than for mice treated individually with UNC2371 or anti-CTLA4. FIGS. 4 to 15 illustrate the data (tumor volume (mm³)) from each individual mouse over the time course of the experiment.

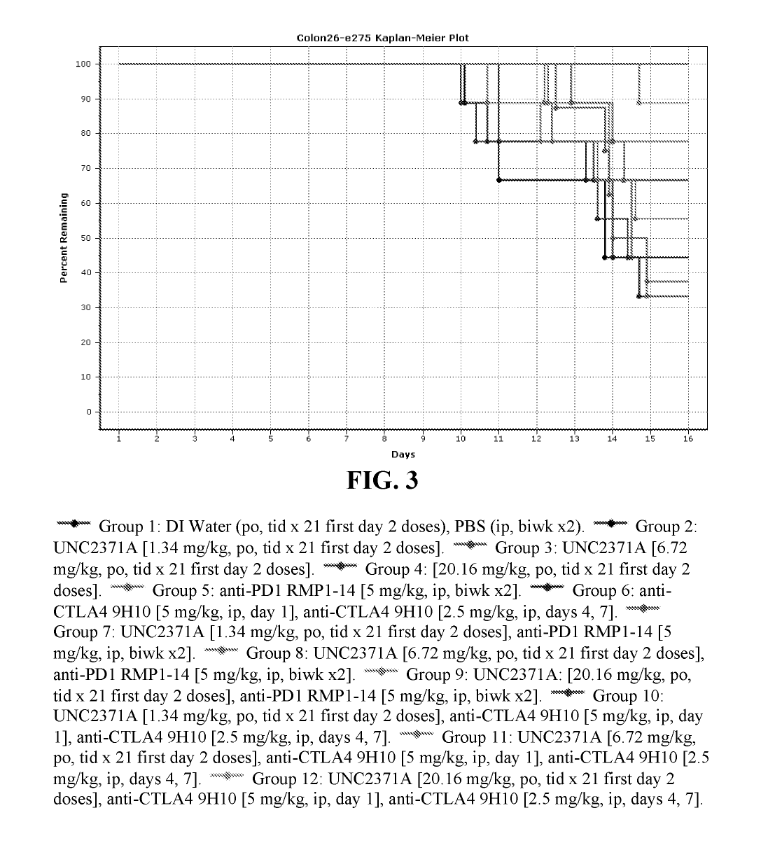

As seen in FIG. 3, the survival rate for mice treated with UNC2371 in combination with anti-CTLA4 was higher than for mice treated individually with UNC2371 or anti-CTLA4 monotherapy. Through day 15 of the experiment, all mice treated with low dose UNC2371 (1 mg/kg TID) in combination with anti-CTLA4 or high dose UNC2371 (15 mg/kg TID) in combination with anti-CTLA4 were still alive (9/9 mice remaining in each group). In contrast, groups of mice treated with low dose UNC2371 (1 mg/kg TID), high dose UNC2371 (15 mg/kg TID), or anti-CTLA4 monotherapy, had only 3, 8, or 4 mice remaining, respectively (n=9 in each treatment group). Groups of mice treated with mid dose UNC2371 (5 mg/kg TID) in combination with anti-CTLA4 monotherapy had 7 mice remaining, compared to 6 mice remaining in the mid dose UNC2371 (5 mg/kg TID) monotherapy group (n=9 in each treatment group). As seen in FIG. 3, the combination of UNC2371 with anti-PD1 led to survival rates that were similar to treatment with UNC2371 monotherapy alone: low dose UNC2371 (1 mg/kg TID) in combination with anti-PD1 (3/9 mice remaining) vs. low dose UNC2371 (1 mg/kg TID) monotherapy (3/9 mice remaining) vs. anti-PD1 monotherapy (3/9 mice remaining); mid dose UNC2371 (5 mg/kg TID) in combination with anti-PD1 (5/9 mice remaining) vs. mid dose UNC2371 (5 mg/kg TID) monotherapy (6/9 mice remaining); high dose UNC2371 (15 mg/kg TID) in combination with anti-PD1 (8/9 mice remaining) vs. high dose UNC2371 (15 mg/kg TID) monotherapy (8/9 mice remaining).

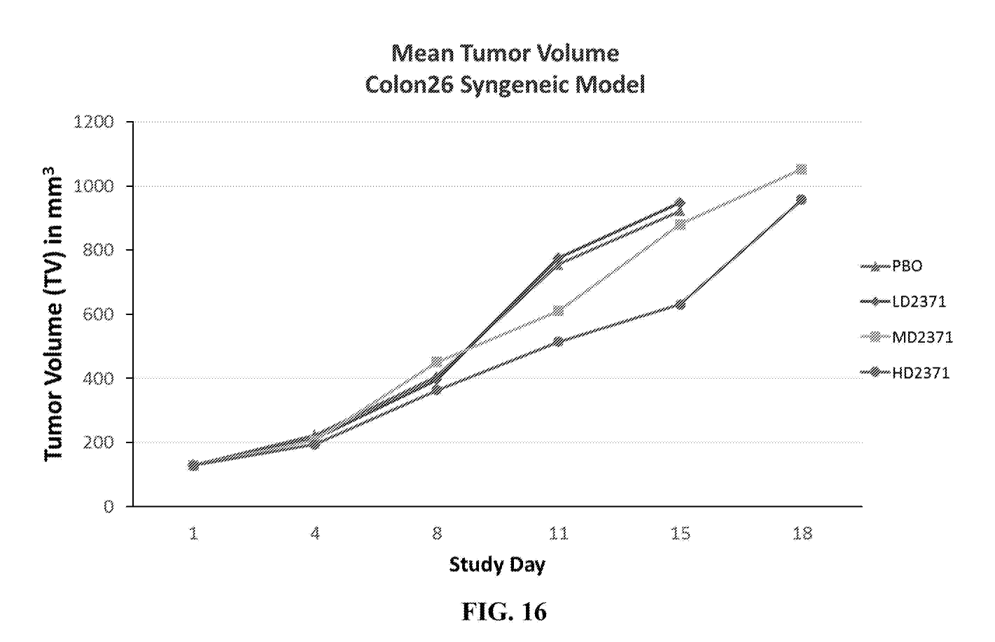

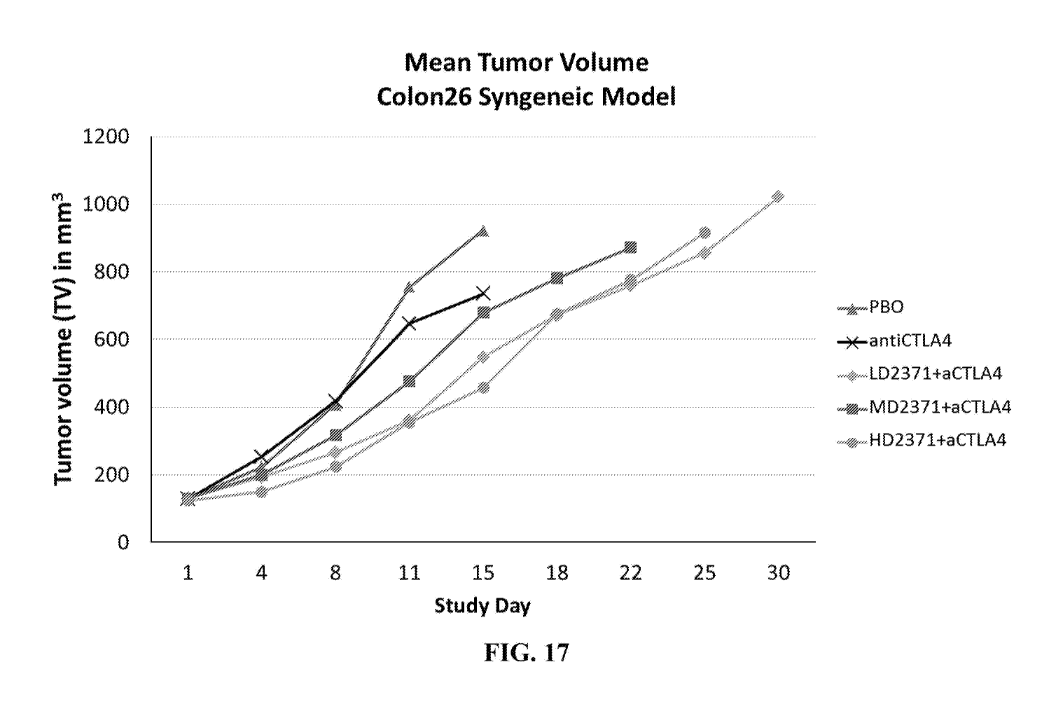

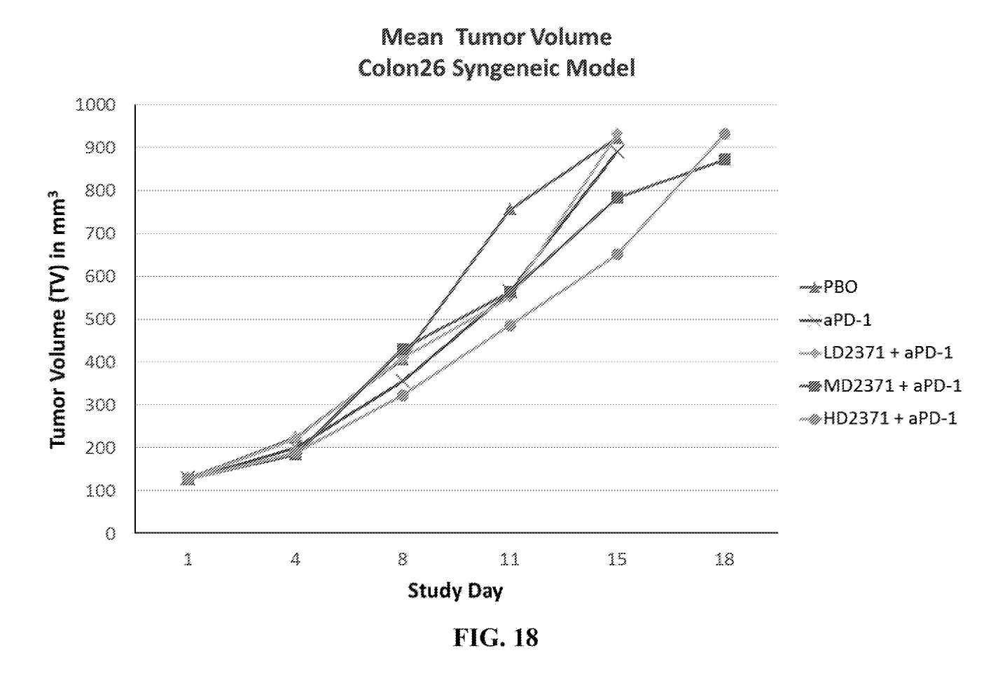

The results from each experimental group up until the experimental endpoint (tumor volume of 1000 mm³ or 45 days, whichever endpoint came first) are summarized in FIGS. 16 to 18. As seen in FIGS. 16 to 18, anti-CTLA4, anti-PDI, and low dose UNC2371 failed to demonstrate single agent efficacy in this colon carcinoma animal model. The median time to endpoint (TTE) for low dose UNC2371 alone was 14.0 days, corresponding to TGD of 0.2 days (1%) (See FIG. 16). The median time to endpoint (TTE) for anti-CTLA4 was 14.4 days, corresponding to tumor growth delay (TGD) of 0.6 days (4%) (See FIG. 17). The median time to endpoint (TTE) for anti-PD1 alone was 14.5 days, corresponding to TGD of 0.7 days (5%) (See FIG. 18).

As seen in FIG. 16, some tumor growth delay (TGD) was observed in the medium and high dose UNC2371 monotherapy arms. The median time to endpoint (TTE) for low dose (1 mg/kg TID), mid dose (5 mg/kg TID) and high dose (15 mg/kg TID) UNC2371 were 14.0, 16.7, and 20.7 days, respectively, corresponding to tumor growth delay of 0.2 days (1%), 2.9 days (21%), and 6.9 days (50%).

As seen in FIG. 17, UNC2371 had a synergistic effect when combined with anti-CTLA4. The median time to endpoint (TTE) for low dose, mid dose, and high dose UNC2371 in combination with anti-CTLA4 was 27.1, 20.7, or 22.3 days, respectively, corresponding to tumor growth delay of 13.3 days (96%), 6.9 days (50%) and 8.5 days (62%).

As seen in FIG. 18, the activity of UNC2371 was enhanced when combined with anti-PD1. The median time to endpoint (TTE) for low dose, mid dose, and high dose UNC2371 in combination with anti-PD1 was 14.5, 15.8, or 17.7 days, respectively, corresponding to tumor growth delay of 0.7 days (5%), 2.0 days (14%) and 3.9 days (28%).

In summary, for all regimens tested, only the UNC2371/anti-CTLA4 combination treatments resulted in statistically superior survival benefit compared to vehicle-treated controls.

Complete responses were observed in the following treatment groups: anti-CTLA4 alone (1), anti-PD1 alone (1), 15 mg/kg UNC2371+anti-PD1 (2), 15 mg/kg UNC2371+anti-CTLA4 (1), and 45 mg/kg UNC2371+anti-CTLA4 (1). All complete responders from this study resisted tumor in a re-challenge study (data not shown).

Tumor growth inhibition (% TGI), defined as the percent difference between the Day 7 median tumor volumes (MTVs) of treated and control mice was included as a secondary evaluation of a treatment effect. The results were analyzed using the Mann-Whitney test, and were deemed statistically significant at P 0.05. The Day 7 MTV for Group 1 controls was 405 mm³, with a range of 405 to 726 mm³. As shown in FIG. 17, tumor growth was inhibited most effectively with the combination of UNC2371+anti-CTLA4. In combination with anti-CTLA-4, UNC2371 doses of 5 mg/kg TID and 15 mg/kg TID produced statistically significant tumor growth inhibition of 33 and 21%, respectively (P<0.05).

### Example 4

### MerTK Inhibitors in Combination with Immune Checkpoint Inhibitors

Tumor samples and blood samples were analyzed by flow cytometry to determine the effect of a MerTK inhibitor on immune cells alone or in combination with an immune checkpoint inhibitor in the Colon26 murine colon carcinoma mouse model. In the following example, the MerTK inhibitor UNC2371 was examined in combination with the immune checkpoint inhibitors anti-CTLA4 and anti-PD1. Experiments were performed as described in Example 3 above, except that mice in each group were euthanized at Day 8, and tumor tissue and blood samples were collected. Blood samples were collected by terminal cardiac puncture under isoflurane anesthesia. Blood was processed for peripheral blood monocyte cells (PBMCs; enriches the white cell population). Flow cytometry was conducted to identify specific white cell populations in both the PBMCs as well as the tumor cells. Tumor samples were preserved (single cell suspension) and maintained at −4° C.

Tumor samples and peripheral blood monocyte cells (PBMCs) were analyzed by flow cytometry for the markers listed in Table 3.

TABLE 3
CD8, MDSC, pan-NK, Treg, M1 and M2
Macrophage Flow Cytometry Markers

| Cell population | Signature marker | Antibody panel |
|---|---|---|
| CD8 | CD3⁺CD8⁺ | CD3, CD8, CD11b, |
| MDSC | CD11B+Gr-1+ | |
| (myeloid-derived | | |
| suppressor cells) | | |
| pan-NK | CD11b^{low}DX5+ | |
| M1 Macrophage | CD3− | CD4, CD25, FoxP3#, |
| | CD68⁺CD80⁺CD206⁻ | CD68, CD80, CD206* |
| M2 Macrophage | CD3−CD68⁺CD80⁻ | |
| | CD206⁺ | |
| Treg | CD3+CD4⁺CD25⁺FoxP3⁺ | |

*CD206, external and internal staining
#FoxP3, internal marker

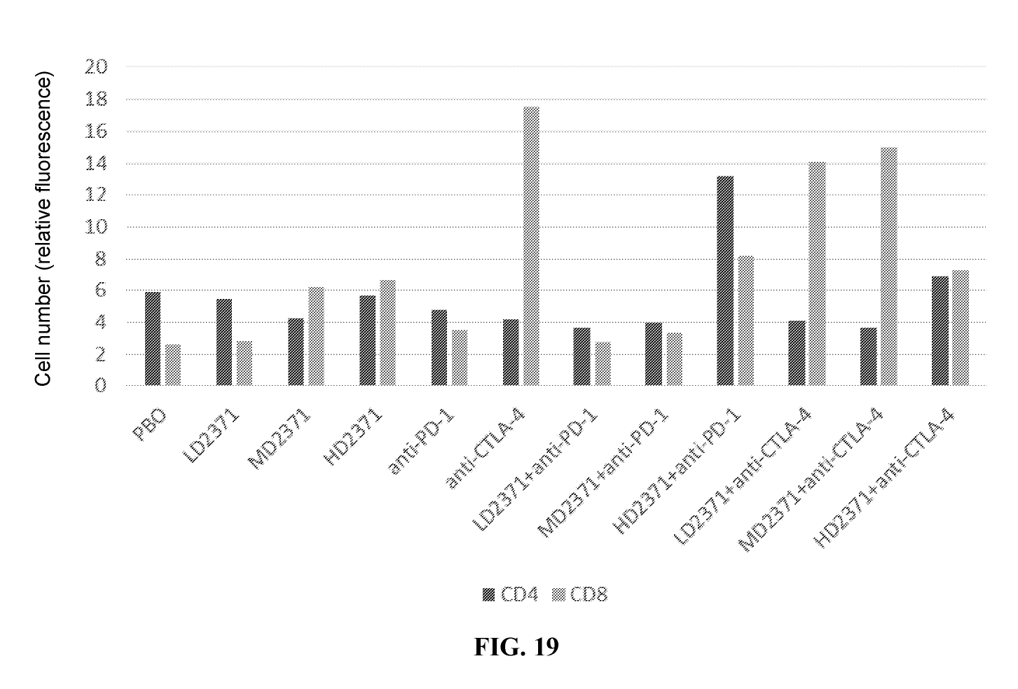

As shown in FIG. 19, UNC2371 treatment was associated with an increase in CD8+ cells in the tumor compared to placebo. Anti-CTLA-4 treatment was associated with the largest CD8+ (cytotoxic lymphocytes) increase, and the combination of UNC2371 (low dose or medium dose) and anti-CTLA4 showed an increase in CD8+ lymphocytes similar to anti-CTLA4 monotherapy.

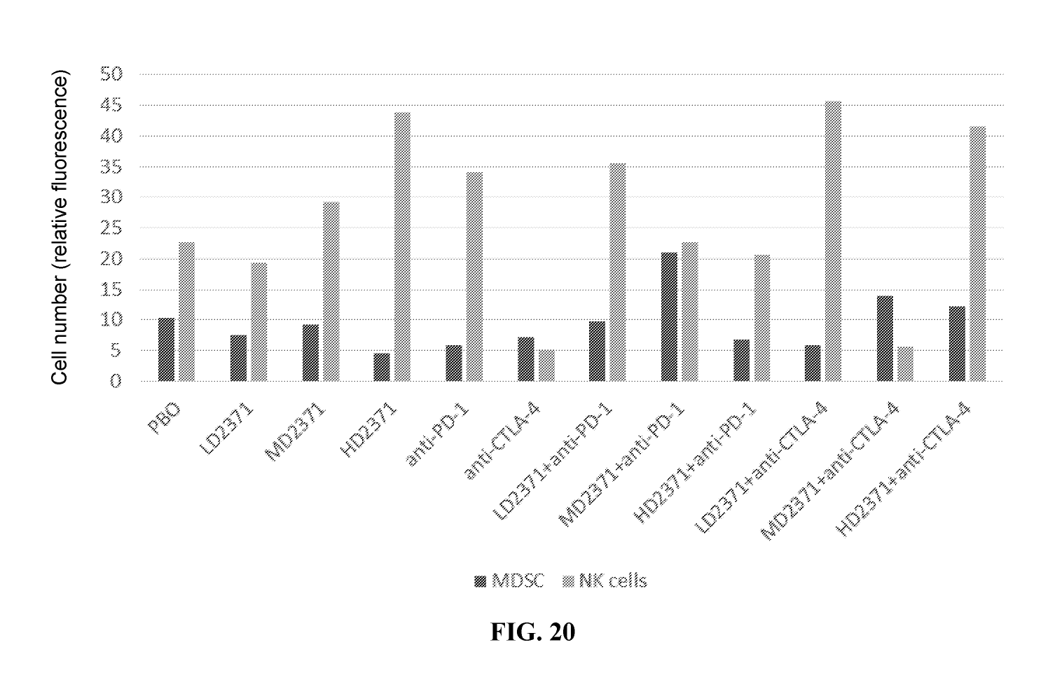

As shown in FIG. 20, there was also an increase in Natural Killer (NK) cells with UNC2371 solo therapy as well as UNC2371/anti-CTLA4 combination treatment. In contrast, anti-CTLA4 treatment alone was associated with a decrease in NK cells in the tumor at Day 8.

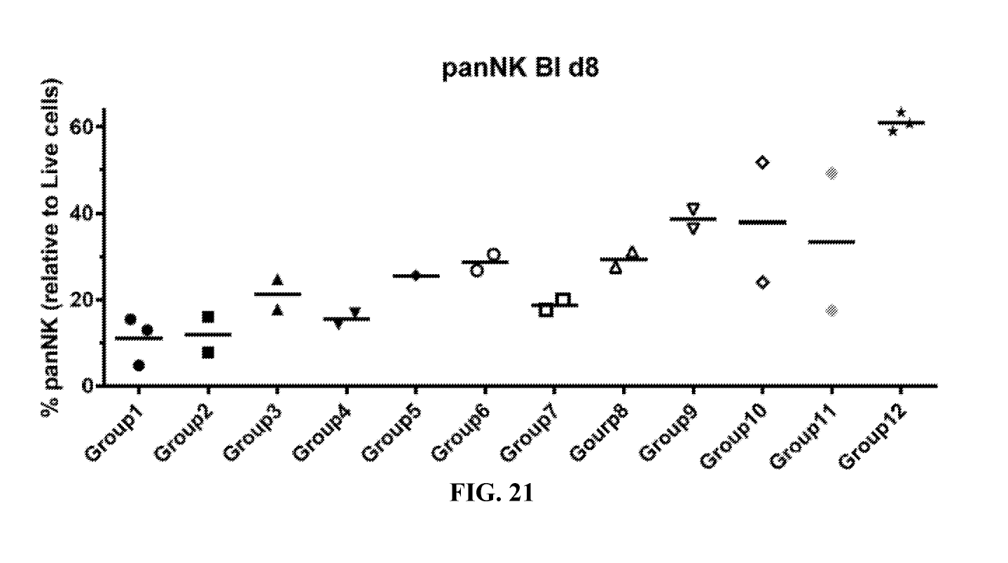

In addition to examining tumor samples, the effect of combining a MerTK inhibitor with an immune checkpoint inhibitor on natural killer (NK) immune cells was also analyzed in peripheral blood samples. As shown in FIG. 21, the combination of UNC2371 and anti-CTLA4 provided the largest increase in the percentage of natural killer (NK) immune cells.

Tumors from 2 to 3 mice per treatment group were formalin fixed and paraffin embedded. The samples were analyzed by immunohistochemistry for several biological markers (data not shown). T cells (cell marker=CD3) were increased in the 1 mg/kg UNC2371 (low dose) monotherapy arm and 1 mg/kg UNC2371/anti-CTLA4 combination treatment arm compared to vehicle. There was also a notable increase in T cell (CD3 marker) penetration into the core of the tumor in UNC2371 treated mice. This is consistent with the increase of CD8+ cells seen by flow cytometry as shown in FIG. 19. Macrophages (cell marker F4/80) were highest in group 6 (anti-CTLA4) and group 10 (UNC2371 1 mg/kg+anti-CTLA4) with similar staining seen in these two groups. All tumors possessed a predominant F4/80 periphery, consistent with what others have reported for F4/80 on tumors. However, Groups 6 (anti-CTLA4) and group 10 (UNC2371 1 mg/kg+anti-CTLA4) also had strongly positive cells extending deeper into the tumors (data not shown). Infiltration of white cells into the tumor (rather than peripheral to tumor) suggests an anti-tumor response. Tumors were also analyzed for survivin staining. Survivin is a protein which inhibits apoptosis or programmed cell death, is upregulated in cancers, and is a marker that is suggestive of direct anti-tumor activity. Analysis of tumors showed markedly decreased staining in group 2 (low dose 1 mg/kg UNC2371 and Group 10 (low dose 1 mg/kg UNC2371 TID+CTLA) (data not shown).

### Example 5

### Analysis of MerTK Inhibitors and Immune Checkpoint Inhibitors in a B16 Melanoma Mouse Model

MerTK ihibitors were analyzed in combination with immune checkpoint inhibitors in a B16 melanoma mouse model for their effect on secondary tumor growth in mice treated with radiation therapy (XRT). XRT is performed according to the following schematic:

Briefly, XRT (8 Gy) was given on days 9, 10, and 11. UNC2371 was given at a dose of 50 mg/kg, by oral gavage, qd, initiated at Day 8. Anti-PD1 was given at 200 μg IP on days 6, 9, and 12. Eight mice were analyzed in each treatment arm (XRT, MerTKi, anti-PD1, anti-PD1+MerTKi, XRT+MerTKi, XRT+anti-PD1, and XRT+anti-PD1+MerTKi). Tumors were implanted on both flanks, and the secondary tumor was shielded during XRT treatment. The secondary tumor was assessed for impact of abscopal effect.

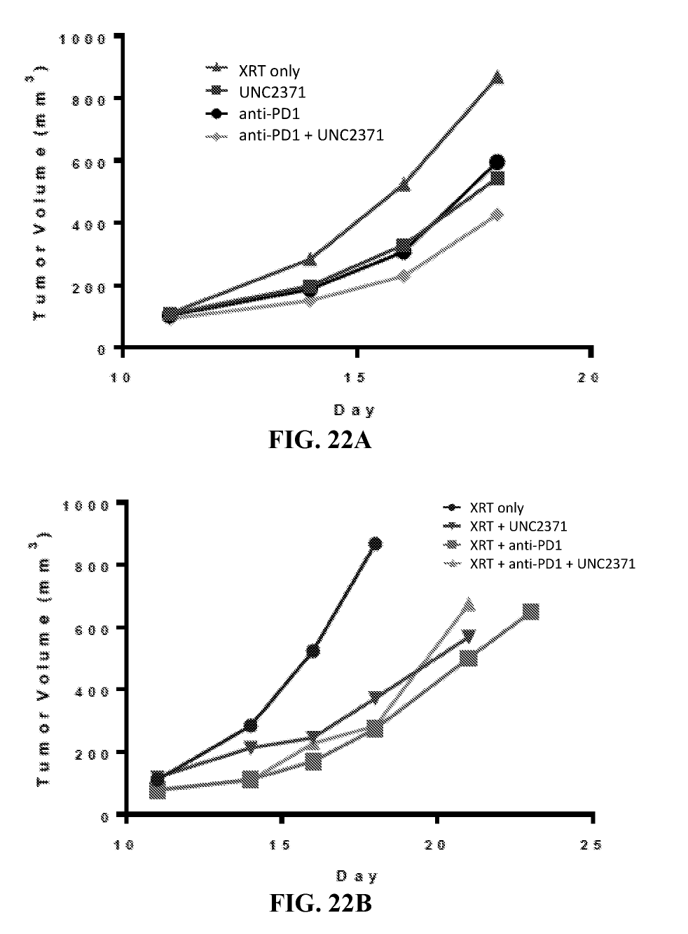

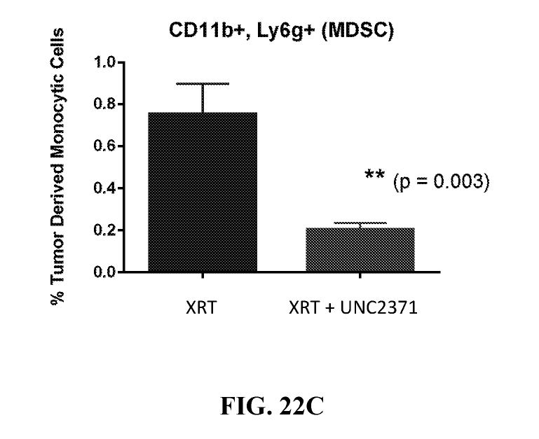

As shown in FIGS. 22A and 22B, all drug treatment arms were more effective than XRT alone. In addition, all drug treatment arms enhanced the abscopal effect on the secondary tumor. As shown in FIG. 22C, there was a decrease in tumor derived myeloid-derived suppressor cells (MDSCs) in mice treated with UNC2371. XRT was 100% effective in inhibiting primary tumor growth, and therefore it was not possible to assess drug treatments on the primary tumor (data not shown).

To evaluate the effect of MerTK inhibitors on myeloid-derived suppressor cells (MDSCs), C57BL/6 B16/F10 melanoma cells were implanted into syngeneic mice and tumors are allowed to form. Once tumors reached 5 mm in diameter, mice received either vehicle (0.9% NaCl) or 10, 25 or 50 mg/kg UNC2371 for 5 days. Initial experiments determined the lowest dose capable of altering the immune system. Myeloid-derived suppressor cells (MDSCs) were isolated from the spleens of tumor-bearing mice by fluorescence activated cell sorting using the cell surface markers CD11b⁺Ly6G⁻Ly6C^{high} and G-MDSCs by CD11b⁺Ly6G^{high}Ly6C^{low}. MDSCs' suppressive capabilities were assessed by the level of RNA for M2 capabilities.

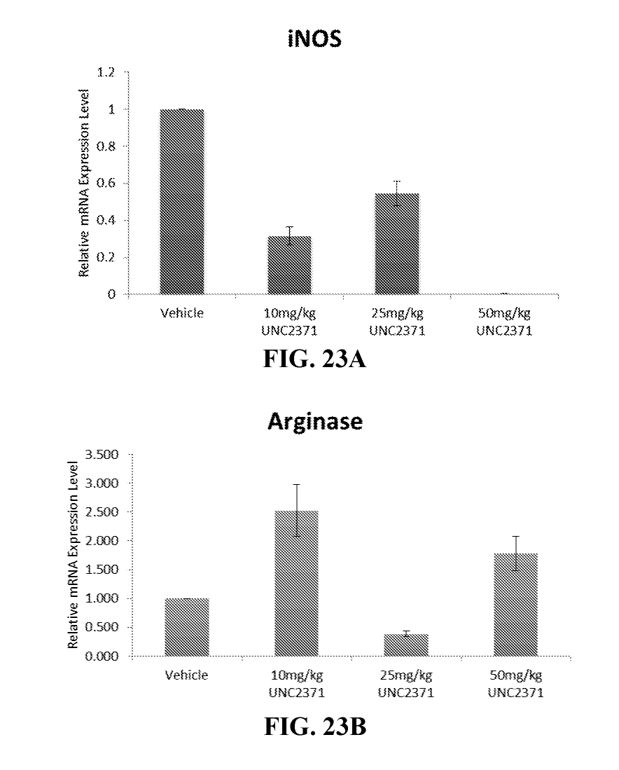

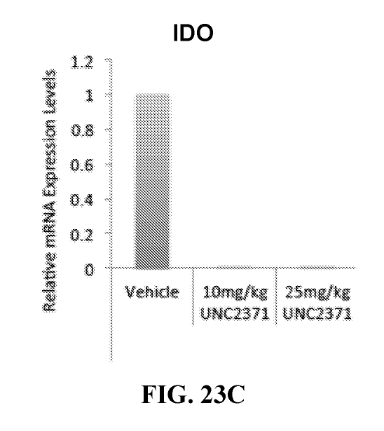

As shown in FIG. 23A, there was a decrease in iNOS (inducible nitric oxide synthase) mRNA expression in mice treated with 10, 25, or 50 mg/kg UNC2371 as compared to vehicle control. As shown in FIG. 23B, there was a decrease in arginase mRNA expression in mice treated with 25 mg/kg UNC2371 as compared to vehicle control. As shown in FIG. 23C, there was a decrease in IDO (indoleamine 2,3-dioxygenase) mRNA expression in mice treated with 10 or 25 mg/kg UNC2371 as compared to vehicle control.

This specification has been described with reference to embodiments of the invention. The invention has been described with reference to assorted embodiments, which are illustrated by the accompanying Examples. The invention can, however, be embodied in different forms and should not be construed as limited to the embodiments set forth herein. Given the teaching herein, one of ordinary skill in the art will be able to modify the invention for a desired purpose and such variations are considered within the scope of the invention.

1-93. (canceled)

94. A method of treating a cancer in a host comprising administering to the host a therapeutically effective combination of a compound of Formula I and an immune checkpoint inhibitor, wherein Formula I has the structure: 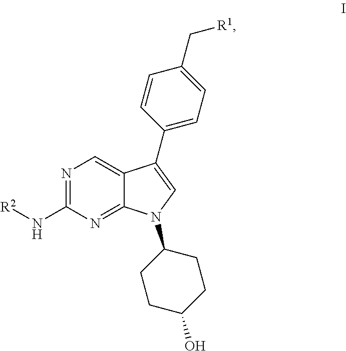
wherein;
R¹ is heterocycle, wherein R¹ is optionally substituted one, two, or three times; and
R² is alkyl, cycloalkyl, or cycloalkylalkyl, wherein R² is optionally substituted one, two, or three times;
or a pharmaceutically acceptable salt thereof.

95. The method of claim 94, wherein the compound of Formula I has the structure: 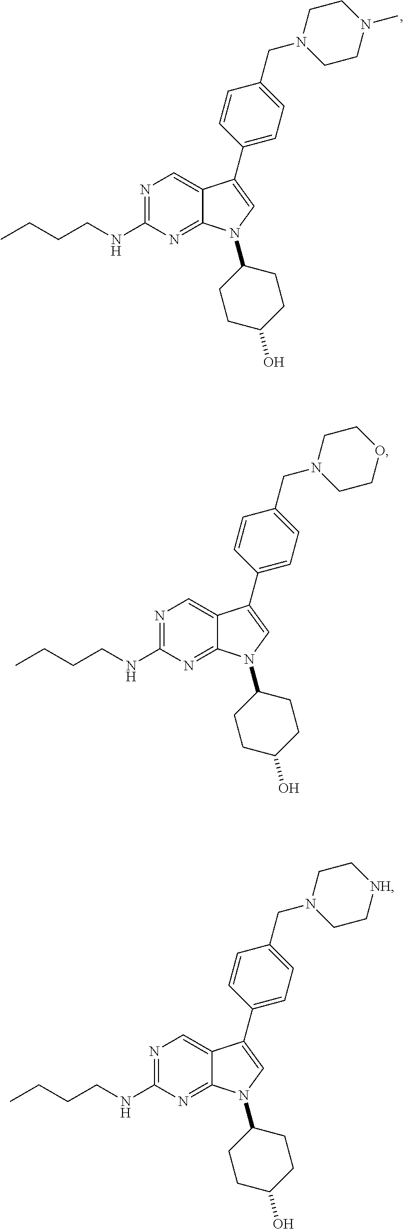 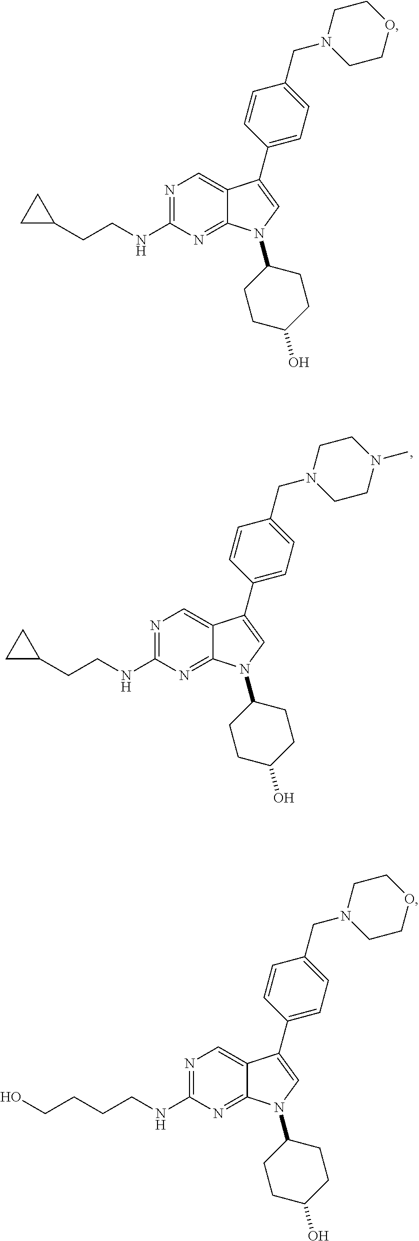 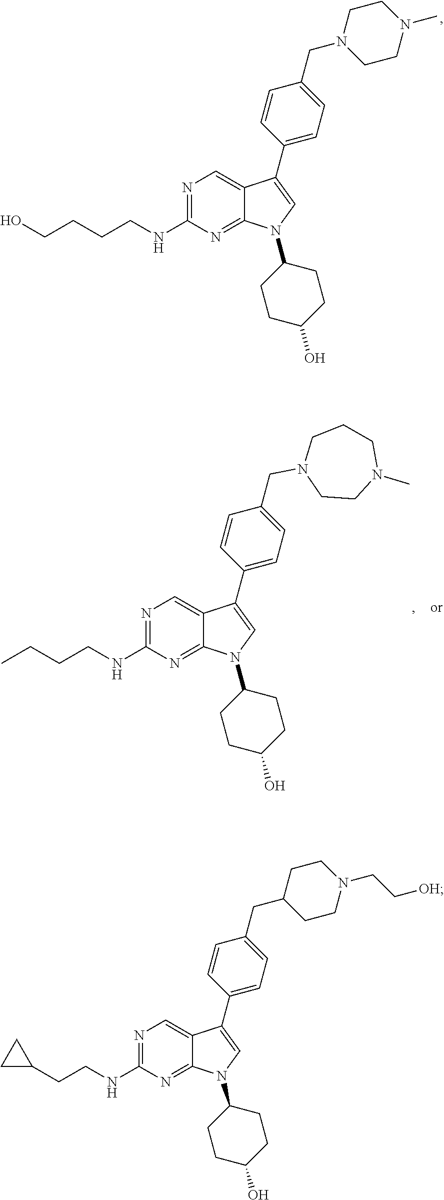
or a pharmaceutically acceptable salt thereof.

96. The method of claim 95, wherein the compound of Formula I is: 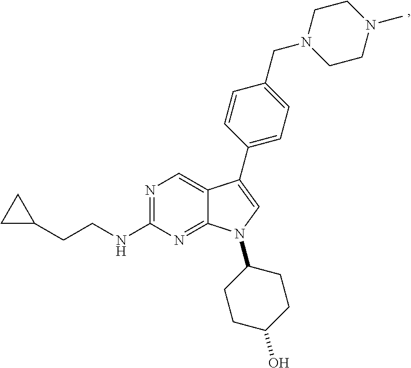
or a pharmaceutically acceptable salt thereof.

97. The method of claim 94, wherein the immune checkpoint inhibitor is selected from a cytotoxic T-lymphocyte-associated protein 4 (CTLA4) inhibitor, a programmed cell death protein 1 (PD1) inhibitor, or a programmed death-ligand 1 (PDL-1) inhibitor.

98. The method of claim 97, wherein the immune checkpoint inhibitor is a cytotoxic T-lymphocyte-associated protein 4 (CTLA4) inhibitor.

99. The method of claim 98, wherein the cytotoxic T-lymphocyte-associated protein 4 (CTLA4) inhibitor is an antibody.

100. The method of claim 99, wherein the antibody is ipilimumab.

101. The method of claim 97, wherein the immune checkpoint inhibitor is a programmed cell death protein 1 (PD1) inhibitor.

102. The method of claim 101, wherein the programmed cell death protein 1 (PD1) inhibitor is an antibody.

103. The method of claim 102, wherein the antibody is nivolumab or pembrolizumab.

104. The method of claim 97, wherein the immune checkpoint inhibitor is a programmed death-ligand 1 (PDL-1) inhibitor.

105. The method of claim 104, wherein the programmed death-ligand 1 (PDL-1) inhibitor is an antibody.

106. The method of claim 94, wherein the cancer is at least one of colon cancer, prostate cancer, lung cancer, breast cancer, or melanoma.

107. The method of claim 94, wherein the dose administered for the compound of Formula I is a subtherapeutic dose when administered alone or the dose administered for the immune checkpoint inhibitor is a subtherapeutic dose when administered alone.

108. The method of claim 94, wherein the dose administered for the compound of Formula I and the dose administered for the immune checkpoint inhibitor are both subtherapeutic doses when administered alone.

109. The method of claim 94, further comprising administering in combination or alternation a Toll-like receptor (TLR) agonist.

110. The method of claim 109, wherein the Toll-like receptor (TLR) agonist is selected from the group consisting of BCG, MPL, and imiquimod.

111. The method of claim 94, further comprising administering in combination or alternation ionizing radiation.

112. A method of treating a cancer in a host comprising administering to the host a therapeutically effective combination of a compound of Formula I and an immune checkpoint inhibitor, wherein the cancer is not responsive to immune checkpoint inhibitor monotherapy, wherein Formula I has the structure: 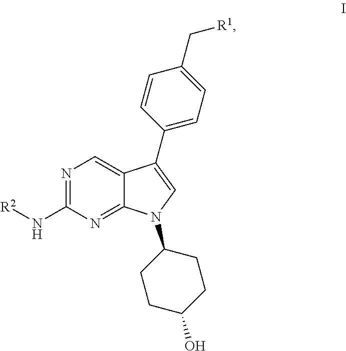
wherein;
R¹ is heterocycle, wherein R¹ is optionally substituted one, two, or three times; and
R² is alkyl, cycloalkyl, or cycloalkylalkyl, wherein R² is optionally substituted one, two, or three times;
or a pharmaceutically acceptable salt thereof.

113. A method of treating a cancer in a host comprising administering to the host a therapeutically effective combination of a compound of Formula I and a Toll-like receptor (TLR) agonist, wherein Formula I has the structure: 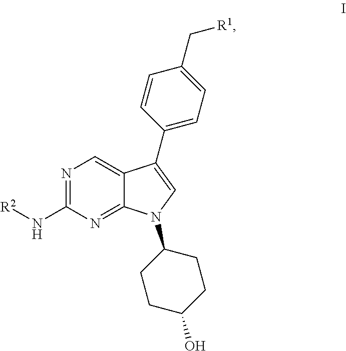
wherein;
R¹ is heterocycle, wherein R¹ is optionally substituted one, two, or three times; and
R² is alkyl, cycloalkyl, or cycloalkylalkyl, wherein R² is optionally substituted one, two, or three times;
or a pharmaceutically acceptable salt thereof.
